SEQUENCE LISTING (75 sequences)

SEQ ID NO: 1 - protein, 5 aa, synthetic construct
SYAIS

SEQ ID NO: 2 - protein, 17 aa, synthetic construct
GIIPIFGTANYAQKFQG

SEQ ID NO: 3 - protein, 10 aa, synthetic construct
GYQGMGVFDP

SEQ ID NO: 4 - protein, 13 aa, synthetic construct
SGSSSNIGSNTVN

SEQ ID NO: 5 - protein, 7 aa, synthetic construct
SNNQRPS

SEQ ID NO: 6 - protein, 11 aa, synthetic construct
AAWDALDWVVV

SEQ ID NO: 7 - protein, 119 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGYQGMGVFDPWGQGTLVTVSS

SEQ ID NO: 8 - protein, 110 aa, synthetic construct
QSVLTQPPSASGTPGQRVTISCSGSSSNIGSNTVNWYQQLPGTAPKLLIYSNNQRPSGVPDRFSGSKSGTSASLAISGLQSEDEADYYCAAWDALDWVVVFGGGTKLTVL

SEQ ID NO: 9 - protein, 245 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGYQGMGVFDPWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDALDWVVVFGGGTKLTVL

SEQ ID NO: 10 - protein, 734 aa, synthetic construct
MPLLLLLPLLWAGALAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGYQGMGVFDPWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDALDWVVVFGGGTKLTVLESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREFEGSAAAEGRGSLLTCGDVEENPGPSGMESDESGLPAMEIECRITGTLNGVEFELVGGGEGTPKQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAFARSRAQSSNSAVDGTAGPGSTGSR

SEQ ID NO: 11 - protein, 5 aa, synthetic construct
SYAIS

SEQ ID NO: 12 - protein, 17 aa, synthetic construct
GIIPIFGTANYAQKFQG

SEQ ID NO: 13 - protein, 10 aa, synthetic construct
GYVQYGYFDY

SEQ ID NO: 14 - protein, 13 aa, synthetic construct
SGSSSNIGSNTVN

SEQ ID NO: 15 - protein, 7 aa, synthetic construct
SNNQRPS

SEQ ID NO: 16 - protein, 10 aa, synthetic construct
AAWDTVDYVV

SEQ ID NO: 17 - protein, 119 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGYVQYGYFDYWGQGTLVTVSS

SEQ ID NO: 18 - protein, 109 aa, synthetic construct
QSVLTQPPSASGTPGQRVTISCSGSSSNIGSNTVNWYQQLPGTAPKLLIYSNNQRPSGVPDRFSGSKSGTSASLAISGLQSEDEADYYCAAWDTVDYVVFGGGTKLTVL

SEQ ID NO: 19 - protein, 244 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGYVQYGYFDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDTVDYVVFGGGTKLTVL

SEQ ID NO: 20 - protein, 733 aa, synthetic construct
MPLLLLLPLLWAGALAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGYVQYGYFDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDTVDYVVFGGGTKLTVLESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREFEGSAAAEGRGSLLTCGDVEENPGPSGMESDESGLPAMEIECRITGTLNGVEFELVGGGEGTPKQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAFARSRAQSSNSAVDGTAGPGSTGSR

SEQ ID NO: 21 - protein, 5 aa, synthetic construct
SYAIS

SEQ ID NO: 22 - protein, 17 aa, synthetic construct
GIIPIFGTANYAQKFQG

SEQ ID NO: 23 - protein, 10 aa, synthetic construct
GFIRYGVFDY

SEQ ID NO: 24 - protein, 13 aa, synthetic construct
SGSSSNIGSNTVN

SEQ ID NO: 25 - protein, 7 aa, synthetic construct
SNNQRPS

SEQ ID NO: 26 - protein, 11 aa, synthetic construct
AAWDDQRLHVV

SEQ ID NO: 27 - protein, 119 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGFIRYGVFDYWGQGTLVTVSS

SEQ ID NO: 28 - protein, 110 aa, synthetic construct
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDQRLHVVFGGGTKLTVL

SEQ ID NO: 29 - protein, 245 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGFIRYGVFDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDDQRLHVVFGGGTKLTVL

SEQ ID NO: 30 - protein, 734 aa, synthetic construct
MPLLLLLPLLWAGALAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGFIRYGVFDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDDQRLHVVFGGGTKLTVLESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREFEGSAAAEGRGSLLTCGDVEENPGPSGMESDESGLPAMEIECRITGTLNGVEFELVGGGEGTPKQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAFARSRAQSSNSAVDGTAGPGSTGSR

SEQ ID NO: 31 - protein, 5 aa, synthetic construct
SYAIS

SEQ ID NO: 32 - protein, 17 aa, synthetic construct
GIIPIFGTANYAQKFQG

SEQ ID NO: 33 - protein, 10 aa, synthetic construct
GYPRYGVFDY

SEQ ID NO: 34 - protein, 13 aa, synthetic construct
SGSSSNIGSNTVN

SEQ ID NO: 35 - protein, 7 aa, synthetic construct
SNNQRPS

SEQ ID NO: 36 - protein, 11 aa, synthetic construct
AAWDRYRFHVV

SEQ ID NO: 37 - protein, 119 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGYPRYGVFDYWGQGTLVTVSS

SEQ ID NO: 38 - protein, 110 aa, synthetic construct
QSVLTQPPSASGTPGQRVTISCSGSSSNIGSNTVNWYQQLPGTAPKLLIYSNNQRPSGVPDRFSGSKSGTSASLAISGLQSEDEADYYCAAWDRYRFHVVFGGGTKLTVL

SEQ ID NO: 39 - protein, 245 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGYPRYGVFDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDRYRFHVVFGGGTKLTVL

SEQ ID NO: 40 - protein, 734 aa, synthetic construct
MPLLLLLPLLWAGALAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGYPRYGVFDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDRYRFHVVFGGGTKLTVLESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREFEGSAAAEGRGSLLTCGDVEENPGPSGMESDESGLPAMEIECRITGTLNGVEFELVGGGEGTPKQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAFARSRAQSSNSAVDGTAGPGSTGSR

SEQ ID NO: 41 - protein, 5 aa, synthetic construct
SYAIS

SEQ ID NO: 42 - protein, 17 aa, synthetic construct
GIIPIFGTANYAQKFQG

SEQ ID NO: 43 - protein, 10 aa, synthetic construct
GFIRYGVFDY

SEQ ID NO: 44 - protein, 13 aa, synthetic construct
SGSSSNIGSNTVN

SEQ ID NO: 45 - protein, 7 aa, synthetic construct
SNNQRPS

SEQ ID NO: 46 - protein, 11 aa, synthetic construct
AAWDDRFHHVV

SEQ ID NO: 47 - protein, 119 aa, synthetic construct; identical to SEQ ID NO: 27

SEQ ID NO: 48 - protein, 110 aa, synthetic construct
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDRFHHVVFGGGTKLTVL

SEQ ID NO: 49 - protein, 245 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGFIRYGVFDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDDRFHHVVFGGGTKLTVL

SEQ ID NO: 50 - protein, 734 aa, synthetic construct
MPLLLLLPLLWAGALAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGFIRYGVFDYWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDDRFHHVVFGGGTKLTVLESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREFEGSAAAEGRGSLLTCGDVEENPGPSGMESDESGLPAMEIECRITGTLNGVEFELVGGGEGTPKQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAFARSRAQSSNSAVDGTAGPGSTGSR

SEQ ID NO: 51 - DNA, 1362 bp, synthetic construct
  source 1..1362: mol_type = other DNA
atgcctttgttgctgctgctgcctcttctttgggctggcgcgctagcccaggttcagcttgttcaatctggcgccgaagtgaagaaacccggcagctctgtgaaggtgtcctgcaaagctagcggcggcacctttagcagctacgccatctcttgggtccgacaggctcctggacaaggcctggaatggatgggcggcatcatccctatcttcggcaccgccaattacgcccagaaattccagggcagagtgaccatcaccgccgacgagtctacaagcaccgcctacatggaactgagcagcctgagaagcgaggacaccgccgtgtactattgcgccagaggctatcaaggcatgggcgtgttcgatccttggggccagggaacactggtcacagtttctagcggaggcggaggatctggtggcggaggaagtggcggaggcggttctgctcaatctgtgctgacacagcctcctagcgcctctggaacacctggccagagagtgacaatcagctgtagcggcagcagctccaacatcggcagcaacaccgtgaactggtatcagcagctgcctggcacagcccctaaactgctgatctacagcaacaaccagcggcctagcggcgtgcccgatagattttctggcagcaagagcggcacaagcgccagcctggctatttctggactgcagagcgaggacgaggccgactattattgtgccgcctgggatgccctggactgggtcgtcgtttttggcggcggaacaaagctgaccgtgctcgagagcaaatatggaccaccatgccctccatgtcctttctgggtgctcgtggttgttggcggagtgctggcctgttacagcctgctggttaccgtggccttcatcatcttttgggtccgaagcaagcggagcagactgctgcacagcgactacatgaacatgacccctagacggcccggacctaccagaaagcactaccagccttacgctcctcctagagacttcgccgcctacagaagccgcgtgaagttttctcgaagtgccgatgctcctgcatatcagcagggacagaaccagctgtacaacgagctgaatctgggccggagagaggaatacgacgtgctggataagaggcgcggcagagacccagaaatgggcgggaagccacgacggaaaaacccccaggaggggctgtataatgaactgcagaaggacaaaatggccgaggcttacagcgaaatcgggatgaagggagagagaaggcgcggaaaaggccacgatggactgtatcagggcctgagcactgccaccaaggacacctacgatgctctgcacatgcaggcactgccacccaggtaa

SEQ ID NO: 52 - protein, 453 aa, synthetic construct
MPLLLLLPLLWAGALAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGYQGMGVFDPWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDALDWVVVFGGGTKLTVLESKYGPPCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - DNA, 585 bp, synthetic construct
  source 1..585: mol_type = other DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaagaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagtaa

SEQ ID NO: 54 - protein, 194 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 55 - DNA, 2007 bp, synthetic construct
  source 1..2007: mol_type = other DNA
atgcctttgttgctgctgctgcctcttctttgggctggcgcgctagcccaggttcagcttgttcaatctggcgccgaagtgaagaaacccggcagctctgtgaaggtgtcctgcaaagctagcggcggcacctttagcagctacgccatctcttgggtccgacaggctcctggacaaggcctggaatggatgggcggcatcatccctatcttcggcaccgccaattacgcccagaaattccagggcagagtgaccatcaccgccgacgagtctacaagcaccgcctacatggaactgagcagcctgagaagcgaggacaccgccgtgtactattgcgccagaggctatcaaggcatgggcgtgttcgatccttggggccagggaacactggtcacagtttctagcggaggcggaggatctggtggcggaggaagtggcggaggcggttctgctcaatctgtgctgacacagcctcctagcgcctctggaacacctggccagagagtgacaatcagctgtagcggcagcagctccaacatcggcagcaacaccgtgaactggtatcagcagctgcctggcacagcccctaaactgctgatctacagcaacaaccagcggcctagcggcgtgcccgatagattttctggcagcaagagcggcacaagcgccagcctggctatttctggactgcagagcgaggacgaggccgactattattgtgccgcctgggatgccctggactgggtcgtcgtttttggcggcggaacaaagctgaccgtgctcgagagcaaatatggaccaccatgccctccatgtcctttctgggtgctcgtggttgttggcggagtgctggcctgttacagcctgctggttaccgtggccttcatcatcttttgggtccgaagcaagcggagcagactgctgcacagcgactacatgaacatgacccctagacggcccggacctaccagaaagcactaccagccttacgctcctcctagagacttcgccgcctacagaagccgcgtgaagttttctcgaagtgccgatgctcctgcatatcagcagggacagaaccagctgtacaacgagctgaatctgggccggagagaggaatacgacgtgctggataagaggcgcggcagagacccagaaatgggcgggaagccacgacggaaaaacccccaggaggggctgtataatgaactgcagaaggacaaaatggccgaggcttacagcgaaatcgggatgaagggagagagaaggcgcggaaaaggccacgatggactgtatcagggcctgagcactgccaccaaggacacctacgatgctctgcacatgcaggcactgccacccaggggatccggagagggcagaggaagtcttctaacatgcggtgacgtggaggagaatcccggccctatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaagaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagtaa

SEQ ID NO: 56 - protein, 668 aa, synthetic construct
MPLLLLLPLLWAGALAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGYQGMGVFDPWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDALDWVVVFGGGTKLTVLESKYGPPCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 57 - skipped

SEQ ID NO: 58 - protein, 11 aa, synthetic construct
  VARIANT 5: note = Any amino acid which may be absent
  VARIANT 6: note = Any amino acid which may be absent
  VARIANT 7: note = Any amino acid which may be absent
  VARIANT 8: note = Any amino acid which may be absent
  VARIANT 9: note = Any amino acid which may be absent
AAWDXXXXXVV

SEQ ID NO: 59 - DNA, 25 bp, synthetic construct
  source 1..25: mol_type = other DNA
cagctatgaccatgattacgaattt

SEQ ID NO: 60 - DNA, 22 bp, synthetic construct
  source 1..22: mol_type = other DNA
ctgatcatcagggtgtccttgg

SEQ ID NO: 61 - DNA, 20 bp, synthetic construct
  source 1..20: mol_type = other DNA
ccgtcgccgccaccatggac

SEQ ID NO: 62 - DNA, 20 bp, synthetic construct
  source 1..20: mol_type = other DNA
ctagaaggcacagtcgaggc

SEQ ID NO: 63 - DNA, 20 bp, synthetic construct
  source 1..20: mol_type = other DNA
ggagctgtatcatcctcttc

SEQ ID NO: 64 - DNA, 21 bp, synthetic construct
  source 1..21: mol_type = other DNA
gagatgctactggggcaacgg

SEQ ID NO: 65 - DNA, 21 bp, synthetic construct
  source 1..21: mol_type = other DNA
ttcgttttctgttctgcgccg

SEQ ID NO: 66 - DNA, 22 bp, synthetic construct
  source 1..22: mol_type = other DNA
tgtacagcagcttctttctgcc

SEQ ID NO: 67 - DNA, 20 bp, synthetic construct
  source 1..20: mol_type = other DNA
agccgtacatgaactgaggg

SEQ ID NO: 68 - protein, 717 aa, synthetic construct
MPLLLLLPLLWAGALAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGYQGMGVFDPWGQGTLVTVSSGGGGSGGGGSGGGGSAQSVLTQPPSASGTPGQRVTISCSGSSSNIGSNTVNWYQQLPGTAPKLLIYSNNQRPSGVPDRFSGSKSGTSASLAISGLQSEDEADYYCAAWDALDWVVVFGGGTKLTVLESKYGPPCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREFEGSAAAEGRGSLLTCGDVEENPGPSGMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 69 - protein, 19 aa, synthetic construct
ATNFSLLKQAGDVEENPGP

SEQ ID NO: 70 - protein, 20 aa, synthetic construct
QCTNYALLKLAGDVESNPGP

SEQ ID NO: 71 - protein, 22 aa, synthetic construct
VKQTLNFDLLKLAGDVESNPGP

SEQ ID NO: 72 - protein, 18 aa, synthetic construct
EGRGSLLTCGDVEENPGP

SEQ ID NO: 73 - protein, 113 aa, unidentified
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 74 - protein, 112 aa, unidentified
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 15 aa, synthetic construct
  VARIANT 1..15: note = This sequence may encompass 1-3 GGGGS repeating units
GGGGSGGGGSGGGGS

(19) United States
(12) Patent Application Publication
(10) Pub. No.: US 2024/0132587 A1
(43) Pub. Date: Apr. 25, 2024

(54) COMPOSITIONS AND METHODS OF TREATING CANCER WITH CHIMERIC ANTIGEN RECEPTORS TARGETING CLAUDIN 18.2

(71) Applicant: MEDIMMUNE, LLC, GAITHERSBURG, MD (US)

(72) Inventors: ALLISON MARIE BARRETT, GAITHERSBURG, MD (US); ZACHARY THOMAS BRITTON, GAITHERSBURG, MD (US); SHANNON BREEN, WILMINGTON, DE (US); ROSA CARRASCO, WILMINGTON, DE (US); CHUNNING YANG, GAITHERSBURG, MD (US); GORDON MOODY, WILMINGTON, DE (US); AMANDA HATKE, WILMINGTON, DE (US)

(21) Appl. No.: 18/481,725

(22) Filed: Oct. 4, 2023

Related U.S. Application Data
(60) Provisional application No. 63/414,799, filed on Oct. 10, 2022.

(51) Int. Cl. C07K 16/28; A61K 39/00; A61P 35/00; C07K 14/705; C07K 14/71; C07K 14/725; C07K 14/73; C12N 5/0783; C12N 15/86

(52) U.S. Cl. CPC C07K 16/28; A61K 39/4631; A61K 39/464402; A61P 35/00; C07K 14/7051; C07K 14/70514; C07K 14/70517; C07K 14/70521; C07K 14/71; C12N 5/0636; C12N 15/86; C07K 2317/622; C07K 2319/03; C12N 2740/15043

(57) ABSTRACT

This disclosure relates to compositions and methods for treating cancer using chimeric antigen receptor T cells and/or antigen binding domains targeting CLDN18.2.

### REFERENCE TO AN ELECTRONIC SEQUENCE LISTING

The contents of the electronic sequence listing (CLDN18CAR-100_SL; Size: 79,936 bytes; and Date of Creation: Jan. 5, 2024) is herein incorporated by reference in its entirety.

### BACKGROUND OF DISCLOSURE

### Field of Invention

This disclosure relates to treatment of cancer using chimeric antigen receptor T cells.

### Technical Background

Chimeric antigen receptor (CAR) T cell therapy is a specific form of cell-based immunotherapy that uses engineered T cells to fight cancer. In CAR T cell therapy, T cells are harvested directly from a patient's blood (autologous) or from modified donor derived cells (allogeneic), engineered ex vivo to express CARs containing both antigen-binding and T cell-activating domains (e.g. comprising one or more co-stimulatory domains), expanded into a larger population, and administered to the patient. The CAR T cells act as a living drug, binding to cancer cells and bringing about their destruction. When successful, the effects of CAR T cell treatment tend to be long lasting, as evidenced by detection of CAR T cell persistence and expansion in the patients long after clinical remission.

The antigen-binding domain of a CAR is an extracellular region that targets a surface antigen on tumor cells. Appropriate target antigens can be proteins, phosphorylated proteins, peptide-WIC, carbohydrates, or glycolipid molecules. Ideal target antigens are widely expressed on tumor cells to enable targeting of a high percentage of the cancer cells and exhibit restricted expression on normal tissues to limit off-tumor toxicity. The antigen-binding domain of a CAR is responsible for directing T cell-mediated cytotoxicity and is commonly composed of one or more antibody or antibody-like targeting moieties, such as an antibody single chain variable fragment (scFv), with specificity for the intended target.

The T cell-activating domain of a CAR is intracellular and activates the T cell in response to the interactions of antigen and antigen-binding domain. A T cell activating domain can contain one or more costimulatory domains, which are the intracellular domains of known activating T cell receptors essential for driving the secondary signal to a CAR-T on antigen engagement. Incorporation of intracellular T cell receptor costimulatory domains (e.g. domains from CD28 or 4-1BB), enhanced proliferation and cytokine secretion of the CAR-Ts. CAR-Ts can also incorporate additional modifications such as multiple costimulatory domains or ability to secrete cytokines to enhance CAR-T cell persistence. The selection and positioning of costimulatory domains within a CAR construct influence CAR T cell function and fate, as costimulatory domains have differential impacts on CAR T cell kinetics, cytotoxic function, and safety profile.

The extracellular antigen-binding and intracellular T cell-activating domains of CARs are linked by a transmembrane domain, hinge, and optionally a spacer region. The hinge domain is a short peptide fragment that provides conformational freedom to facilitate binding to the target antigen on the tumor cell. It may be used alone or in conjunction with a spacer domain that projects the scFv away from the T cell surface. The optimal length of the spacer depends on the proximity of the binding epitope to the cell surface. CAR-T design can also encompass modifications to the transmembrane and hinge regions, lending to CAR-Ts with altered persistence and reactivity to low antigen expressing cells (Majzner Cancer Discov, 2020 May; 10(5):702-72).

It has been several years since first approval of CAR T therapy for use against the B-lymphocyte antigen CD19 (Kymriah®, Novartis) and this and other CD19 CAR-Ts have since shown promising clinical efficacy in pediatric acute lymphocytic leukemia, relapsed or refractory non-Hodgkin lymphoma, and diffuse large B-cell lymphoma (DLBCL) (J Hematol Oncol Pharm. 2022; 12(1):30-42). With first the demonstrated clinical efficacy of CAR-T cells targeting B-cell maturation antigen (BCMA) against relapsed/refractory multiple myeloma with Abecma and followed up by approval of Carvykti in 2022, there are now 6 approved CAR-T products on market (Leukemia volume 36, pages1481-1484, 2022).

More recent data suggest that the CAR approach can be efficacious against solid tumors. A disialoganglioside 2 (GD2) CAR natural killer T cell (NKT) therapy has shown activity in neuroblastoma (Heczey A, Nature Medicine volume 26, pages1686-1690, 2020). Additionally, GD2 CAR-T have demonstrated clinical efficacy and a manageable toxicity profile in pediatric neuroblastoma patients (Journal of Cancer Research and Clinical Oncology (2022) 148:2643-2652). CAR-T therapy targeting mesothelin in combination with pembrolizumab demonstrated anti-tumor activity and safety in patients with malignant pleural mesothelioma (Cancer Discov, 2021, Nov. 11 (11):2748-2763). Interim analysis of a phase I clinical trial of CLDN18.2 targeting CAR-Ts demonstrated these CAR-T were well tolerated and had promising antitumor efficacy compared to other therapeutic approaches utilized in the third line setting of gastric cancer (Nature Medicine volume 28, pages1189-1198 (2022)). Other clinical studies are underway evaluating safety and efficacy of CAR-T therapies in a variety of solid tumor indications including several clinical trials of CAR-Ts to GPC3 (hepatocellular carcinoma, Front. Oncol., 16 Feb. 2022), CLDN6 (testicular cancer, J Immunother Cancer 2021; 9(Suppl 2):A1-A1054) and PSMA (metastatic castrate resistant prostate cancer, Nat Med 2022 Apr., 28(4):724-34). However, identification of new targets, optimized CAR-T design and manufacturing, are needed to succeed in the solid tumor setting.

Unfortunately, the complexities of CAR T cell-based therapy can lead to undesirable and unsafe effects. Off-tumor effects such as neurotoxicity and acute respiratory distress syndrome are potential adverse effects of CAR T cell therapy and are potentially fatal. Cytokine release syndrome (CRS) is the most common acute toxicity associated with CAR T cells. CRS occurs when lymphocytes are highly activated and release excessive amounts of inflammatory cytokines. Serum elevations of interleukin 2, interleukin 6, interleukin 1 beta, GM-CSF, and/or C-reactive protein are sometimes observed in patients with CRS when these factors are assayed. CRS is graded in severity and is diagnosed as one of grades 1-4 (mild to severe), with more serious cases clinically characterized by high fever, hypotension, hypoxia, and/or multi-organ toxicity. One study reported that 92% of acute lymphocytic leukemia patients treated with an anti-CD19 CAR T cell therapy experienced CRS, and 50% of these patients developed grade 3-4 symptoms.

Therefore, additional CAR T cell-based therapies are needed to augment the armamentarium of effective cancer treatments especially in solid tumor settings. However, new CAR T cell therapies must be devised that effectively treat cancer while minimizing the risk of developing dangerous inflammatory responses, such as CRS.

### BRIEF SUMMARY

This disclosure describes compositions and methods for using CAR T cells to treat cancer.

As described below, in a first aspect, the disclosure provides an isolated nucleic acid sequence encoding a chimeric antigen receptor (CAR), wherein the CAR comprises:
(a) an antigen binding domain specific for claudin 18.2 (CLDN18.2);
(b) a transmembrane domain; and
(c) one or more intracellular domains.

In some embodiments of the isolated nucleic acid sequence, the antigen binding domain comprises an antibody or antigen-binding fragment thereof, Fab, Fab′, F(ab′)2, Fd, Fv, single-chain fragment variable (scFv), single chain antibody, VHH, vNAR, nanobody (single-domain antibody), or any combination thereof. In certain embodiments, the antigen binding domain is a single chain variable fragment (scFv).

In some embodiments of the isolated nucleic acid sequence, the antigen binding domain is a scFv comprising an amino acid sequence selected from SEQ ID NO: 9, 19, 29, 39, and 49.

In some embodiments of the isolated nucleic acid sequence, transmembrane domain comprises a transmembrane domain selected from the transmembrane domain of CD4, CD8α, or CD28. In certain embodiments, the transmembrane domain comprises a CD28 transmembrane domain.

In some embodiments of the isolated nucleic acid sequence, the one or more intracellular domains comprises a costimulatory domain or a portion thereof. In certain embodiments, the costimulatory domain comprises one or more of CD3z, CD2, CD27, CD28, 4-1BB, OX-40, ICOS, IL-2Rβ, GITR, MyD88/CD40a costimulatory domains, and/or variants thereof. In an embodiment, the intracellular domain comprises a CD3z costimulatory domain and a CD28 costimulatory domain. In an embodiment, the intracellular domain comprises a CD3z costimulatory domain and a 4-1BB costimulatory domain. In an embodiment, the intracellular domain comprises a CD3z costimulatory domain, a CD28 costimulatory domain, and a 4-1BB costimulatory domain.

In some embodiments of the isolated nucleic acid sequence, the CAR further comprises a hinge/spacer domain, optionally, wherein the hinge/spacer domain is located between the antigen binding domain and the transmembrane domain. In certain embodiments, the hinge/spacer domain comprises an IgG1 hinge domain or variants thereof, an IgG2 hinge domain or variants thereof, an IgG3 hinge domain or variants thereof, an IgG4 hinge domain or variants thereof, an IgG4P domain, a CD8 hinge domain or variants thereof or a CD28 hinge domain or variants thereof. In certain embodiments, the hinge/spacer domain is an IgG4 hinge/spacer, or variants thereof, optionally an IgG4P hinge/spacer comprising an S241P mutation.

In some embodiments of the isolated nucleic acid sequence, the nucleic acid sequence encodes a CAR with an amino acid sequence as set forth in SEQ ID NO: 52, optionally wherein the nucleic acid sequence is as set forth in SEQ ID NO: 51.

In some embodiments of the isolated nucleic acid sequence, the nucleic acid sequence further comprises an armoring domain comprising a nucleic acid sequence encoding an armoring molecule, optionally wherein the armoring domain is located at the 3′ end of the nucleic acid encoding the CAR or at the 5′ end of the nucleic acid encoding the CAR. In certain embodiments, the armoring molecule is selected from a dominant-negative TGFβ receptor type II, IL-7, IL-12, IL-15, IL-18, a hybrid IL-4/IL-7 receptor, a hybrid IL-7/IL-2 receptor, and HIF1α dominant-negative. In an embodiment, the armoring molecule comprises a dominant-negative TGFβ receptor type II (dnTGFβRII). In certain embodiments, the armoring molecule comprises an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO: 54. In an embodiment, the dominant negative TGFβ receptor type II comprises the sequence of SEQ ID NO:54, optionally wherein the armoring domain encoding the dnTGFβRII has the sequence as set forth in SEQ ID NO: 53.

In some embodiments of the isolated nucleic acid sequence, the CAR and the armoring domain are operably linked under the control of a single promoter. In some embodiments of the isolated nucleic acid sequence, the CAR and the armoring domain are operably linked by an internal ribosome entry site (IRES). In some embodiments of the isolated nucleic acid sequence, the CAR and the armoring domain are linked by a nucleotide sequence encoding a cleavable peptide linker. In certain embodiments, the cleavable peptide linker is a self-cleaving peptide linker. In an embodiment, the cleavable peptide linker comprises a T2A peptide.

In some embodiments of the isolated nucleic acid sequence, the nucleic acid sequence encodes a sequence selected from SEQ ID NO: 55, 10, 20, 30, 40, and 50.

In a second aspect, the disclosure provides an anti-CLDN18.2 chimeric antigen receptor (CAR) comprising an antigen binding domain, wherein the antigen binding domain comprises an antibody, Fab, or an scFv comprising a heavy chain variable region (VH) and a light chain variable region (VL);
wherein the VH comprises a CDR1 comprising an amino acid sequence selected from SEQ ID NO: 1, 11, 21, 31, and 41; a CDR2 comprising an amino acid sequence selected from SEQ ID NO: 2, 12, 22, 32, and 42; and a CDR3 comprising an amino acid sequence selected from SEQ ID NO: 3 13, 23, 33, and 43; and
wherein the VL comprises a CDR1 comprising an amino acid sequence selected from SEQ ID NO: 4, 14, 24, 34, and 44; a CDR2 comprising an amino acid sequence selected from SEQ ID NO: 5, 15, 25, 35, and 45; and a CDR3 comprising an amino acid sequence selected from SEQ ID NO: 6, 16, 26, 36, and 46.

In some embodiments of the anti-CLDN18.2 CAR, the VH comprises an amino acid sequence selected from SEQ ID NO: 7, 17, 27, 37 and 47.

In some embodiments of the anti-CLDN18.2 CAR, the VL comprises an amino acid sequence selected from SEQ ID NO: 8, 18, 28, 38, and 48.

In some embodiments of the anti-CLDN18.2 CAR, the CAR comprises a transmembrane domain, and one or more intracellular domains. In certain embodiments, the transmembrane domain comprises a transmembrane domain selected from the transmembrane domain of CD4, CD8α, or CD28. In an embodiment, the transmembrane domain comprises a CD28 transmembrane domain.

In some embodiments of the anti-CLDN18.2 CAR, the one or more intracellular domains comprises a costimulatory domain or a portion thereof. In certain embodiments, the costimulatory domain comprises one or more of CD3z, CD2, CD27, CD28, 4-1BB, OX-40, ICOS, IL-2Rβ, GITR, MyD88/CD40a costimulatory domains, and/or variants thereof. In an embodiment, the intracellular domain comprises a CD3z costimulatory domain and a CD28 costimulatory domain. In an embodiment, the intracellular domain comprises a CD3z costimulatory domain and a 4-1BB costimulatory domain. In an embodiment, the intracellular domain comprises a CD3z costimulatory domain, a CD28 costimulatory domain, and a 4-1BB costimulatory domain.

In some embodiments of the anti-CLDN18.2 CAR, the CAR further comprises a hinge/spacer domain, optionally, wherein the hinge/spacer domain is located between the antigen binding domain and the transmembrane domain. In certain embodiments, the hinge/spacer domain comprises an IgG1 hinge domain or variants thereof, an IgG2 hinge domain or variants thereof, an IgG3 hinge domain or variants thereof, an IgG4 hinge domain or variants thereof, an IgG4P domain, a CD8a hinge domain or variants thereof, or a CD28 hinge domain or variants thereof. In certain embodiments, the hinge/spacer domain is an IgG4 hinge/spacer, or variants thereof, optionally an IgG4P hinge/spacer comprising an S241P mutation.

In some embodiments of the anti-CLDN18.2 CAR, the CAR has an amino acid sequence as set forth in SEQ ID NO: 52.

In some embodiments of the anti-CLDN18.2 CAR, the CAR further comprises an armoring molecule. In certain embodiments, the armoring molecule is selected from a dominant-negative TGFβ receptor type II, IL-7, IL-12, IL-15, IL-18, a hybrid IL-4/IL-7 receptor, a hybrid IL-7/IL-2 receptor, and HIF1α dominant-negative. In an embodiment, the armoring molecule comprises a dominant-negative TGFβ receptor type II (dnTGFβRII). In certain embodiments, the armoring molecule comprises an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO:54. In an embodiment, the dominant negative TGFβ receptor type II comprises the sequence of SEQ ID NO:54.

In some embodiments of the anti-CLDN18.2 CAR, the CAR and the armoring molecule are linked by a nucleotide sequence encoding a cleavable peptide linker. In certain embodiments, the cleavable peptide linker is a self-cleaving peptide linker. In an embodiment, the cleavable peptide linker comprises a T2A peptide.

In some embodiments of the anti-CLDN18.2 CAR, the CAR comprises an amino acid sequence selected from SEQ ID NO: 56, 10, 20, 30, 40, and 50.

In a third aspect, the disclosure provides a vector comprising the isolated nucleic acid sequence as disclosed herein or encoding the chimeric antigen receptor as disclosed herein, optionally wherein the vector is a virus, a lentivirus, an adenovirus, a retrovirus, an adeno-associated virus (AAV), a transposon, a DNA vector, a mRNA, a lipid nanoparticle (LNP), or a CRISPR-Cas System, optionally wherein the vector is a lentivirus.

In a fourth aspect, the disclosure provides cell comprising the vector as disclosed herein.

In a fifth aspect, the disclosure provides a cell comprising a nucleic acid sequence encoding the chimeric antigen receptor (CAR) as disclosed herein, preferably wherein the cell comprises a nucleic acid sequence encoding a CAR with an amino acid sequence as set forth in SEQ ID NO: 52 and a nucleic acid encoding a dominant negative TGFβ receptor type II with a sequence as set forth in SEQ ID NO:54, optionally wherein the nucleic acid sequence encoding the CAR is as set forth in SEQ ID NO: 51 and the sequence encoding the dominant negative TGFβ receptor type II is as set forth in SEQ ID NO: 53.

In a sixth aspect, the disclosure provides, a cell comprising a CLDN18.2 specific antigen binding domain, wherein the antigen binding domain comprises an antibody, Fab, or an scFv comprising a heavy chain variable region (VH) and a light chain variable region (VL);
wherein the VH comprises a CDR1 comprising an amino acid sequence selected from SEQ ID NO: 1, 11, 21, 31, and 41; a CDR2 comprising an amino acid sequence selected from SEQ ID NO: 2, 12, 22, 32, and 42; and a CDR3 comprising an amino acid sequence selected from SEQ ID NO: 3 13, 23, 33, and 43; and
wherein the VL comprises a CDR1 comprising an amino acid sequence selected from SEQ ID NO: 4, 14, 24, 34, and 44; a CDR2 comprising an amino acid sequence selected from SEQ ID NO: 5, 15, 25, 35, and 45; and a CDR3 comprising an amino acid sequence selected from SEQ ID NO: 6, 16, 26, 36, and 46.

In some embodiments of the cell comprising a CLDN18.2 specific antigen binding domain, the VH comprises an amino acid sequence selected from SEQ ID NO: 7, 17, 27, 37 and 47.

In some embodiments of the cell comprising a CLDN18.2 specific antigen binding domain, the VL comprises an amino acid sequence selected from SEQ ID NO: 8, 18, 28, 38, and 48.

In some embodiments of the cell comprising a CLDN18.2 specific antigen binding domain, the CLDN18.2 specific antigen binding domain comprises the sequence as set forth in SEQ ID NO: 52.

In some embodiments of the cell comprising a CLDN18.2 specific antigen binding domain, the cell further comprises an armoring molecule. In certain embodiments, the armoring molecule is selected from a dominant-negative TGFβ receptor type II, IL-7, IL-12, IL-15, IL-18, a hybrid IL-4/IL-7 receptor, a hybrid IL-7/IL-2 receptor, and HIF1α dominant-negative. In an embodiment, the armoring molecule comprises a dominant-negative TGFβ receptor type II (dnTGFβRII). In certain embodiments, the armoring molecule comprises an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO:54. In an embodiment, the dominant negative TGFβ receptor type II comprises the sequence of SEQ ID NO:54.

In some embodiments of the cell comprising a CLDN18.2 specific antigen binding domain, the cell is selected from a T cell, a Natural Killer (NK) cell, a cytotoxic T lymphocyte (CTL), a tumor infiltrating lymphocyte, and a regulatory T cell.

In some embodiments of the cell comprising a CLDN18.2 specific antigen binding domain, the cell exhibits an anti-tumor immunity upon contacting a tumor cell expressing CLDN18.2.

In a seventh aspect, the disclosure provides a method of treating cancer, comprising:
administering to a subject in need thereof an effective amount of a cell comprising an anti-CLDN18.2 chimeric antigen receptor (CAR) comprising an antigen binding domain, wherein the antigen binding domain comprises an antibody, Fab, or an scFv comprising a heavy chain variable region (VH) and a light chain variable region (VL),
wherein the VH comprises a CDR1 comprising an amino acid sequence selected from SEQ ID NO: 1, 11, 21, 31, and 41; a CDR2 comprising an amino acid sequence selected from SEQ ID NO: 2, 12, 22, 32, and 42; and a CDR3 comprising an amino acid sequence selected from SEQ ID NO: 3 13, 23, 33, and 43; and
wherein the VL comprises a CDR1 comprising an amino acid sequence selected from SEQ ID NO: 4, 14, 24, 34, and 44; a CDR2 comprising an amino acid sequence selected from SEQ ID NO: 5, 15, 25, 35, and 45; and a CDR3 comprising an amino acid sequence selected from SEQ ID NO: 6, 16, 26, 36, and 46.

In some embodiments of the method of treating cancer, the method further comprises inhibiting tumor growth, inducing tumor regression, and/or prolonging survival of the subject.

In some embodiments of the method of treating cancer, the cell is an autologous cell. In certain embodiments, the autologous cell is selected from a T cell, a Natural Killer (NK) cell, a cytotoxic T lymphocyte (CTL), a tumor infiltrating lymphocyte, and a regulatory T cell.

In some embodiments of the method of treating cancer, the cancer is a solid tumor. In certain embodiments, the solid tumor is gastric cancer, gastroesophageal junction cancer (GEJ; e.g., distal oesophageal cancers, proximal gastric cancers and cancers of cardia), pancreatic cancer, breast cancer, colon cancer, liver cancer, head and neck cancer, bronchial cancer, biliary adenocarcinoma, ovarian cancer, hepatocellular carcinoma, or non-small-cell lung cancer. In an embodiment, the solid tumor is pancreatic cancer.

In an eighth aspect, the disclosure provides, an antibody or an antigen-binding portion thereof that specifically binds CLDN18.2, comprising a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3, wherein
(a) the VH-CDR1 comprises an amino acid sequence selected from SEQ ID NOs: 1, 11, 21, 31, and 41;
(b) the VH-CDR2 comprises an amino acid sequence selected from SEQ ID NOs: 2, 12, 22, 32, and 42;
(c) the VH-CDR3 comprises an amino acid sequence selected from SEQ ID NOs: 3, 13, 23, 33, and 43;
(d) the VL-CDR1 comprises an amino acid sequence selected from SEQ ID NOs: 4, 14, 24, 34, and 44;
(e) the VL-CDR2 comprises an amino acid sequence selected from SEQ ID NOs: 5, 15, 25, 35, and 45; and
(f) the VL-CDR3 comprises an amino acid sequence selected from SEQ ID NOs: 6, 16, 26, 36, and 46.

In some embodiments of the antibody or an antigen-binding portion:
(a) the VH-CDR1 comprises the amino acid sequence set forth in SEQ ID NO: 1, the VH-CDR2 comprises the amino acid sequence set forth in SEQ ID NO: 2, the VH-CDR3 comprises the amino acid sequence set forth in SEQ ID NO: 3, the VL-CDR1 comprises the amino acid sequence set forth in SEQ ID NO: 4, the VL-CDR2 comprises the amino acid sequence set forth in SEQ ID NO: 5, and the VL-CDR3 comprises the amino acid sequence set forth in SEQ ID NO: 6;
(b) the VH-CDR1 comprises the amino acid sequence set forth in SEQ ID NO: 11, the VH-CDR2 comprises the amino acid sequence set forth in SEQ ID NO: 12, the VH-CDR3 comprises the amino acid sequence set forth in SEQ ID NO: 13, the VL-CDR1 comprises the amino acid sequence set forth in SEQ ID NO: 14, the VL-CDR2 comprises the amino acid sequence set forth in SEQ ID NO: 15, and the VL-CDR3 comprises the amino acid sequence set forth in SEQ ID NO: 16;
(c) the VH-CDR1 comprises the amino acid sequence set forth in SEQ ID NO: 21, the VH-CDR2 comprises the amino acid sequence set forth in SEQ ID NO: 22, the VH-CDR3 comprises the amino acid sequence set forth in SEQ ID NO: 23, the VL-CDR1 comprises the amino acid sequence set forth in SEQ ID NO: 24, the VL-CDR2 comprises the amino acid sequence set forth in SEQ ID NO: 25, and the VL-CDR3 comprises the amino acid sequence set forth in SEQ ID NO: 26;
(d) the VH-CDR1 comprises the amino acid sequence set forth in SEQ ID NO: 31, the VH-CDR2 comprises the amino acid sequence set forth in SEQ ID NO: 32, the VH-CDR3 comprises the amino acid sequence set forth in SEQ ID NO: 33, the VL-CDR1 comprises the amino acid sequence set forth in SEQ ID NO: 34, the VL-CDR2 comprises the amino acid sequence set forth in SEQ ID NO: 35, and the VL-CDR3 comprises the amino acid sequence set forth in SEQ ID NO: 36; or
(e) the VH-CDR1 comprises the amino acid sequence set forth in SEQ ID NO: 41, the VH-CDR2 comprises the amino acid sequence set forth in SEQ ID NO: 42, the VH-CDR3 comprises the amino acid sequence set forth in SEQ ID NO: 43, the VL-CDR1 comprises the amino acid sequence set forth in SEQ ID NO: 44, the VL-CDR2 comprises the amino acid sequence set forth in SEQ ID NO: 45, and the VL-CDR3 comprises the amino acid sequence set forth in SEQ ID NO: 46.

In some embodiments of the antibody or an antigen-binding portion, the VH comprises an amino acid sequence having at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 7, 17, 27, 37, and 47.

In some embodiments of the antibody or an antigen-binding portion, the VH comprises an amino acid sequence selected from SEQ ID NOs: 7, 17, 27, 37, and 47.

In some embodiments of the antibody or an antigen-binding portion, the VL comprises an amino acid sequence having at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 8, 18, 28, 38, and 48.

In some embodiments of the antibody or an antigen-binding portion, the VL comprises an amino acid sequence selected from SEQ ID NOs: 8, 18, 28, 38, and 48.

In some embodiments of the antibody or an antigen-binding portion:
(a) the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7, and the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8;
(b) the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17, and the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18;
(c) the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 27, and the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 28;
(d) the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 37, and the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 38; or
(e) the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 47, and the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 48.

In some embodiments of the antibody or an antigen-binding portion:
(a) the VH comprises the amino acid sequence set forth in SEQ ID NO: 7, and the VL comprises the amino acid sequence set forth in SEQ ID NO: 8;
(b) the VH comprises the amino acid sequence set forth in SEQ ID NO: 17, and the VL comprises the amino acid sequence set forth in SEQ ID NO: 18;
(c) the VH comprises the amino acid sequence set forth in SEQ ID NO: 27, and the VL comprises the amino acid sequence set forth in SEQ ID NO: 28;
(d) the VH comprises the amino acid sequence set forth in SEQ ID NO: 37, and the VL comprises the amino acid sequence set forth in SEQ ID NO: 38; or
(e) the VH comprises the amino acid sequence set forth in SEQ ID NO: 47, and the VL comprises the amino acid sequence set forth in SEQ ID NO: 48.

In a ninth aspect, the disclosure provides, a pharmaceutical composition comprising the isolated nucleic acid of any one of claims 1 to 26, the anti-CLDN18.2 CAR of any one of claims 27 to 50, the vector of claim 51, the cell of any one of claims 52 to 64, or the antibody or the antigen-binding portion thereof of any one of claims 72 to 79, and a pharmaceutically acceptable excipient.

In a tenth aspect, the disclosure provides, a method of treating a disease or condition in a subject in need thereof, comprising administering to the subject the isolated nucleic acid as disclosed herein, the anti-CLDN18.2 CAR as disclosed herein, the vector of claim as disclosed herein, the cell as disclosed herein, the antibody or the antigen-binding portion thereof as disclosed herein, or the pharmaceutical composition as disclosed herein. In certain embodiments, the disease or condition comprises a cancer.

In some aspects, the disclosure provides, a method of treating a cancer in a subject in need thereof, comprising administering to the subject the isolated nucleic acid as disclosed herein, the anti-CLDN18.2 CAR as disclosed herein, the vector as disclosed herein, the cell as disclosed herein, the antibody or the antigen-binding portion thereof as disclosed herein, or the pharmaceutical composition as disclosed herein. In certain embodiments, the cancer is gastric cancer, gastroesophageal junction cancer (GEJ; e.g., distal oesophageal cancers, proximal gastric cancers and cancers of cardia), pancreatic cancer, breast cancer, colon cancer, liver cancer, head and neck cancer, bronchial cancer, biliary adenocarcinoma, ovarian cancer, hepatocellular carcinoma, or non-small-cell lung cancer.

In some aspects, the disclosure provides for the use of the isolated nucleic acid as disclosed herein, the anti-CLDN18.2 CAR as disclosed herein, the vector as disclosed herein, the cell as disclosed herein, the antibody or the antigen-binding portion thereof as disclosed herein, or the pharmaceutical composition as disclosed herein in the treatment of a disease or condition in a subject in need thereof. In certain embodiments, the disease or condition comprises a cancer. In some aspects, the disclosure provides for the use of the isolated nucleic acid as disclosed herein, the anti-CLDN18.2 CAR as disclosed herein, the vector as disclosed herein, the cell as disclosed herein, the antibody or the antigen-binding portion thereof as disclosed herein, or the pharmaceutical composition as disclosed herein in the treatment of a cancer in a subject in need thereof. In certain embodiments, the cancer is gastric cancer, gastroesophageal junction cancer (GEJ; e.g., distal oesophageal cancers, proximal gastric cancers and cancers of cardia), pancreatic cancer, breast cancer, colon cancer, liver cancer, head and neck cancer, bronchial cancer, biliary adenocarcinoma, ovarian cancer, hepatocellular carcinoma, or non-small-cell lung cancer.

In an eleventh aspect the disclosure provides a method of expanding a population of T cells comprising:
(a) isolating CD3⁺ T cells from a sample;
(b) culturing the CD3⁺ T cells in a culture media that comprises human interleukin 21 (IL-21);
(c) activating the CD3⁺ T cells;
(d) transducing the CD3⁺ T cells with a vector comprising a nucleic acid encoding a chimeric antigen receptor (CAR) that binds CLDN18.2 to produce CAR-T cells;
(e) culturing the CAR-T cells in a medium; and
(f) harvesting the CAR-T cells.

In twelfth aspect, the disclosure provides a method of manufacturing a T cell therapeutic comprising:
(a) obtaining a sample comprising a population of CD3⁺ T cells;
(b) culturing the CD3⁺ T cells in a culture media that comprises human interleukin 21 (IL-21);
(c) activating the CD3⁺ T cells;
(d) transducing the CD3⁺ T cells with a vector comprising a nucleic acid encoding a chimeric antigen receptor (CAR) that binds CLDN18.2 to produce CAR-T cells;
(e) culturing the CAR-T cells or T-cell Receptor (TCR) cells in a medium; and
(f) harvesting the CAR-T cells.

In some embodiments of the method of expanding and/or manufacturing a T cell population, the method of either claim 89 or 90, wherein the population of CD3+ T cells is formed from an isolated population of CD4+ and CD8+ T cells.

In some embodiments of the method of expanding and/or manufacturing a T cell population, the culture media further comprises human interleukin 2 (IL-2).

In some embodiments of the method of expanding and/or manufacturing a T cell population, about from 1×106 to about 1×109 CD3+ T cells are cultured in step (b) in the culture media.

In some embodiments of the method of expanding and/or manufacturing a T cell population, the sample is an enriched apheresis product collected via leukapheresis.

In some embodiments of the method of expanding and/or manufacturing a T cell population, the CD3+ T cells in step (c) are cultured for about one day or about two days.

In some embodiments of the method of expanding and/or manufacturing a T cell population, the CD3+ T cells in step (c) are activated with agonists of CD2, CD3, CD28, or any combination thereof.

In some embodiments of the method of expanding and/or manufacturing a T cell population, the CD3+ T cells in step (c) are activated with magnetic microbeads.

In some embodiments of the method of expanding and/or manufacturing a T cell population, the CD3+ T cells in step (c) are activated with an anti-CD3 antibody or CD3-binding fragment thereof, and an anti-CD28 antibody or a CD28-binding fragment thereof.

In some embodiments of the method of expanding and/or manufacturing a T cell population, the anti-CD3 antibody or CD3-binding fragment thereof, and the anti-CD28 antibody or a CD28-binding fragment thereof are coupled to a magnetic microbead.

In some embodiments of the method of expanding and/or manufacturing a T cell population, the CAR-T cells are cultured in step (e) from about two to about ten days.

In some embodiments of the method of expanding and/or manufacturing a T cell population, the CAR-T cells are cultured in step (e) from about four to about six days.

In some embodiments of the method of expanding and/or manufacturing a T cell population, the CAR-T cells are cultured in step (e) for about four days.

In some embodiments of the method of expanding and/or manufacturing a T cell population, the CAR-T cells are cultured in step (e) for about six days.

In some embodiments of the method of expanding and/or manufacturing a T cell population, the concentration of human IL-21 is from about 0.01 U/mL to about 0.3 U/mL, and the concentration of human IL-2 is from about 5 IU/mL to about 100 IU/mL. In an embodiment, the concentration of human IL-21 is about 0.19 U/mL. In an embodiment, the concentration of human IL-2 is about 40 IU/mL.

In some embodiments of the method of expanding and/or manufacturing a T cell population, the CD3+ T cells are agitated during step (b).

In yet another aspect, the disclosure provides, a method of manufacturing a T cell therapeutic comprising: (a) isolating CD4+ and CD8+ T cells from a sample to form a population of CD3+ T cells; (b) culturing the CD3+ T cells in a culture media that comprises human interleukin 2 at a concentration of 40 IU/mL and human interleukin 21 at a concentration of 0.19 U/mL; (c) activating the CD3+ T cells with a magnetic bead comprising an anti-CD3 antibody or CD3-binding fragment thereof, and an anti-CD28 antibody or a CD28-binding fragment thereof; (d) transducing the CD3+ T cells with a vector comprising a nucleic acid encoding a chimeric antigen receptor (CAR) that binds CLDN18.2 to produce CAR-T cells; (e) culturing the CAR-T cells in a medium for about four days; and (f) harvesting the CAR-T cells. In certain embodiments, the vector is a virus, a lentivirus, an adenovirus, a retrovirus, an adeno-associated virus (AAV), a transposon, a DNA vector, a mRNA, a lipid nanoparticle (LNP), or a CRISPR-Cas System. In an embodiment, the vector is a lentivirus. In certain embodiments, the lentivirus is added at a multiplicity of invention (MOI) of about 0.25 to about 20. In an embodiment, the lentivirus is added at a MOI of about 1 to about 4. In another embodiment, the lentivirus is added at a MOI of about 2, or about 4.

In some embodiments of the method of expanding and/or manufacturing a T cell population, the cell culture media is increased in volume after step (d).

In some embodiments of the method of expanding and/or manufacturing a T cell population, the cell culture media is increased in volume at least about 6 fold.

In some embodiments of the method of expanding and/or manufacturing a T cell population, the medium in step (e) is exchanged at least once per day.

In some embodiments of the method of expanding and/or manufacturing a T cell population, the medium in step (e) is exchanged about every 12 hours.

In some embodiments of the method of expanding and/or manufacturing a T cell population, the CAR-T cells are expanded from at least about 1 fold to about 5 fold during step (e). In some embodiments, the CAR-T cells are expanded from at least about 1 fold to about 3 fold during step (e). In an embodiment, the CAR-T cells are expanded about 2 fold during step (e). In yet another embodiment, the CAR-T cells are expanded about 3 fold during step (e).

In some embodiments of the method of expanding and/or manufacturing a T cell population, the CAR that binds CLDN18.2 comprises an antigen-binding domain comprising:
(a) a VH-CDR1 comprises the amino acid sequence set forth in SEQ ID NO: 1, a VH-CDR2 comprises the amino acid sequence set forth in SEQ ID NO: 2, a VH-CDR3 comprises the amino acid sequence set forth in SEQ ID NO: 3, a VL-CDR1 comprises the amino acid sequence set forth in SEQ ID NO: 4, a VL-CDR2 comprises the amino acid sequence set forth in SEQ ID NO: 5, and a VL-CDR3 comprises the amino acid sequence set forth in SEQ ID NO: 6;
(b) a VH-CDR1 comprises the amino acid sequence set forth in SEQ ID NO: 11, a VH-CDR2 comprises the amino acid sequence set forth in SEQ ID NO: 12, a VH-CDR3 comprises the amino acid sequence set forth in SEQ ID NO: 13, a VL-CDR1 comprises the amino acid sequence set forth in SEQ ID NO: 14, a VL-CDR2 comprises the amino acid sequence set forth in SEQ ID NO: 15, and a VL-CDR3 comprises the amino acid sequence set forth in SEQ ID NO: 16;
(c) a VH-CDR1 comprises the amino acid sequence set forth in SEQ ID NO: 21, a VH-CDR2 comprises the amino acid sequence set forth in SEQ ID NO: 22, a VH-CDR3 comprises the amino acid sequence set forth in SEQ ID NO: 23, a VL-CDR1 comprises the amino acid sequence set forth in SEQ ID NO: 24, a VL-CDR2 comprises the amino acid sequence set forth in SEQ ID NO: 25, and a VL-CDR3 comprises the amino acid sequence set forth in SEQ ID NO: 26;
(d) a VH-CDR1 comprises the amino acid sequence set forth in SEQ ID NO: 31, a VH-CDR2 comprises the amino acid sequence set forth in SEQ ID NO: 32, a VH-CDR3 comprises the amino acid sequence set forth in SEQ ID NO: 33, a VL-CDR1 comprises the amino acid sequence set forth in SEQ ID NO: 34, a VL-CDR2 comprises the amino acid sequence set forth in SEQ ID NO: 35, and a VL-CDR3 comprises the amino acid sequence set forth in SEQ ID NO: 36; or
(e) a VH-CDR1 comprises the amino acid sequence set forth in SEQ ID NO: 41, a VH-CDR2 comprises the amino acid sequence set forth in SEQ ID NO: 42, a VH-CDR3 comprises the amino acid sequence set forth in SEQ ID NO: 43, a VL-CDR1 comprises the amino acid sequence set forth in SEQ ID NO: 44, a VL-CDR2 comprises the amino acid sequence set forth in SEQ ID NO: 45, and a VL-CDR3 comprises the amino acid sequence set forth in SEQ ID NO: 46.

In some embodiments of the method of expanding and/or manufacturing a T cell population, the CAR that binds CLDN18.2 comprises a VH comprising an amino acid sequence having at least about 75%, at least about 80%, at least about 85%, at least about 90%, at least about 95%, at least about 96%, at least about 97%, at least about 98%, or at least about 99% sequence identity to the amino acid sequence selected from SEQ ID NOs: 7, 17, 27, 37, and 47.

In some embodiments of the method of expanding and/or manufacturing a T cell population, the CAR that binds CLDN18.2 comprises a VH comprising an amino acid sequence selected from SEQ ID NOs: 7, 17, 27, 37, and 47.

In some embodiments of the method of expanding and/or manufacturing a T cell population, the CAR that binds CLDN18.2 comprises a VL comprising an amino acid sequence having at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 8, 18, 28, 38, and 48.

In some embodiments of the method of expanding and/or manufacturing a T cell population, the CAR that binds CLDN18.2 comprises a VL comprising an amino acid sequence selected from SEQ ID NOs: 8, 18, 28, 38, and 48.

In some embodiments of the method of expanding and/or manufacturing a T cell population, the CAR that binds CLDN18.2 comprises:
(a) a VH comprising an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7, and a VL comprising an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8;
(b) a VH comprising an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17, and a VL comprising an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18;
(c) a VH comprising an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 27, and a VL comprising an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 28;
(d) a VH comprising an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 37, and a VL comprising an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 38; or
(e) a VH comprising an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 47, and a VL comprising an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 48.

In some embodiments of the method of expanding and/or manufacturing a T cell population, the CAR that binds CLDN18.2 comprises:
(a) a VH comprising the amino acid sequence set forth in SEQ ID NO: 7, and a VL comprising the amino acid sequence set forth in SEQ ID NO: 8;
(b) a VH comprising the amino acid sequence set forth in SEQ ID NO: 17, and a VL comprising the amino acid sequence set forth in SEQ ID NO: 18;
(c) a VH comprising the amino acid sequence set forth in SEQ ID NO: 27, and a VL comprising the amino acid sequence set forth in SEQ ID NO: 28;
(d) a VH comprising the amino acid sequence set forth in SEQ ID NO: 37, and a VL comprising the amino acid sequence set forth in SEQ ID NO: 38; or
(e) a VH comprising the amino acid sequence set forth in SEQ ID NO: 47, and a VL comprising the amino acid sequence set forth in SEQ ID NO: 48.

In some embodiments of the method of expanding and/or manufacturing a T cell population, the CAR that binds CLDN18.2 comprises the sequence as set forth in SEQ ID NO: 52.

In some embodiments of the method of expanding and/or manufacturing a T cell population, the nucleic acid encoding the CAR that binds CLDN18.2 further comprises an armoring domain comprising a nucleic acid encoding an armoring molecule, optionally wherein the armoring domain is located at the 3′ end of the nucleic acid encoding the CAR or at the 5′ end of the nucleic acid encoding the CAR.

In some embodiments of the method of expanding and/or manufacturing a T cell population, the CAR-T cells comprise an armoring molecule. In certain embodiments, the armoring molecule is selected from a dominant-negative TGFβ receptor type II, IL-7, IL-12, IL-15, IL-18, a hybrid IL-4/IL-7 receptor, a hybrid IL-7/IL-2 receptor, and HIF1α dominant-negative. In an embodiment, the armoring molecule comprises a dominant-negative TGFβ receptor type II (dnTGFβRII). In some embodiments, the armoring molecule comprises an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO:54. In an embodiment, the dominant negative TGFβ receptor type II comprises the sequence of SEQ ID NO:54.

In some embodiments of the method of expanding and/or manufacturing a T cell population, the CAR-T cells are formulated in an isotonic solution. In certain embodiments, the isotonic solution comprises plasmalyte containing human serum albumin.

In some embodiments of the method of expanding and/or manufacturing a T cell population, the isotonic solution contains between about 1×106 and about 1×109 CAR-T cells.

In some embodiments of the method of expanding and/or manufacturing a T cell population, the isotonic solution contains about 3.4×106 CAR-T cells.

In some embodiments of the method of expanding and/or manufacturing a T cell population, the CAR-T cells are a mixture of TCM and TSCM cells.

In some embodiments of the method of expanding and/or manufacturing a T cell population, from about 15% to about 50% of the CAR-T cells are TSCM cells and express CD45RA, CCR7 and CD27, and do not express CD45RO.

In some embodiments of the method of expanding and/or manufacturing a T cell population, about 20% to about 30% of the CAR-T cells are TSCM cells and express CD45RA, CCR7 and CD27, and do not express CD45RO.

In some embodiments of the method of expanding and/or manufacturing a T cell population, more than 50% of the CAR-T cells express a chimeric antigen receptor.

In some embodiments of the method of expanding and/or manufacturing a T cell population, from about 40% to about 60% of the CAR-T cells express a chimeric antigen receptor.

In some embodiments of the method of expanding and/or manufacturing a T cell population, more than 50% of the CAR-T cells express CD8.

In some embodiments of the method of expanding and/or manufacturing a T cell population, from about 40% to about 60% of the CAR-T cells express CD8.

These and other features and advantages of the present disclosure will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure. The drawings illustrate one or more embodiment(s) of the disclosure, and together with the description serve to explain the principles and operation of the disclosure.

FIGS. 1A-1D show protein expression of CLDN18.2 in normal and cancer tissue. FIG. 1A: Human Protein Atlas overview of CLDN18 tissue expression, which includes CLDN18.1 and CLDN18.2. Note, the elevated expression in the lung is likely due to CLDN18.1. FIG. 1B: IHC (Abcam clone EPR19202) was used to demonstrate CLDN18.2-specific staining of normal tissues of potential reactivity. Constitutive expression was found in all normal stomach human samples stained. Focal staining was observed in Brunner's glands and crypts of the human duodenum. CLDN18.2 staining was also observed in hyperplastic or reactive cells of gallbladder and pancreatic ducts (age-related change), this staining was frequent in gallbladder and rare in pancreas. FIG. 1C: IHC expression of CLDN18.2 was maintained in normal gastric tissue of cynomolgus monkey, rat, and NSG mouse. Expression pattern was conserved across animal species; therefore, this target tissue can be modelled in safety studies. FIG. 1D: CLDN18.2 expression was examined across multiple tumor pieces from gastric (G) and gastro-esophageal junction carcinoma (GEJC), pancreatic adenocarcinoma (PDAC) and esophageal adenocarcinomas (EAC). In total, 75 TMA cores were scored for G/GEJC, 24 cores for PDAC and 17 cores for EAC. Expression and intensity of staining were measured for each sample. The table represents samples with any level of expression.

FIGS. 2A-2D show CLDN18.2 binding affinity and species cross-reactivity of select leads as determined by flow cytometry measurement in HEK293 cells expressing human (FIG. 2A), cyno (FIG. 2B), rat (FIG. 2C) or mouse (FIG. 2D) CLDN18.2. All leads bind human and cyno CLDN18.2 (99% homology), most leads bind rat CLDN18.2 (90% homology) and only one internal lead binds murine CLDN18.2 (89% homology. R347 is used as a negative control.

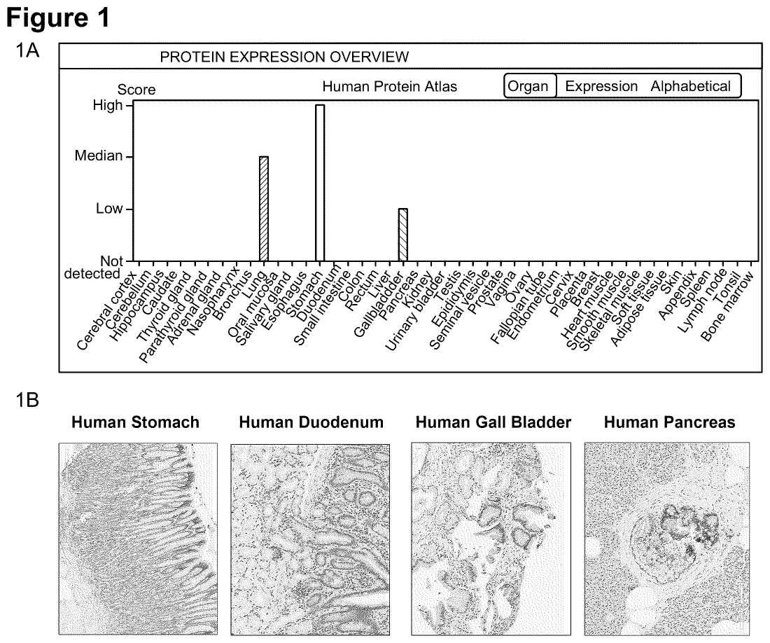

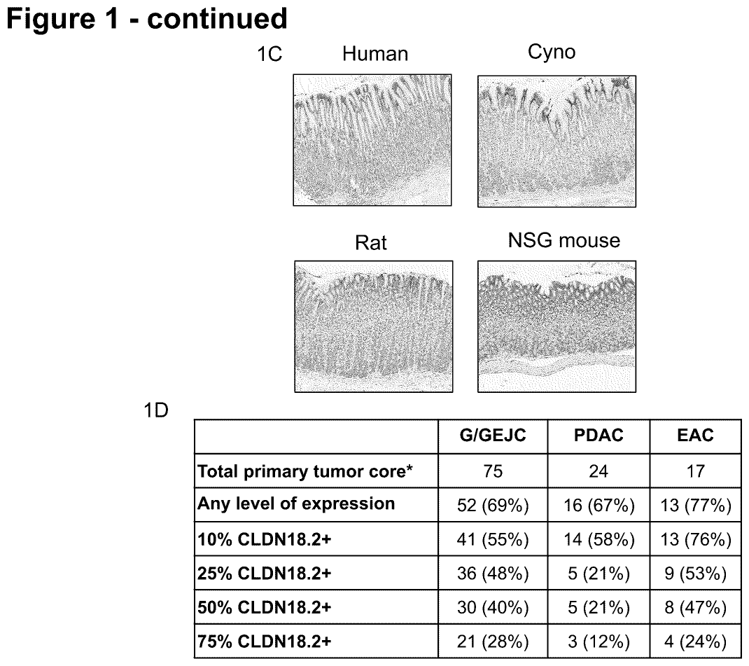

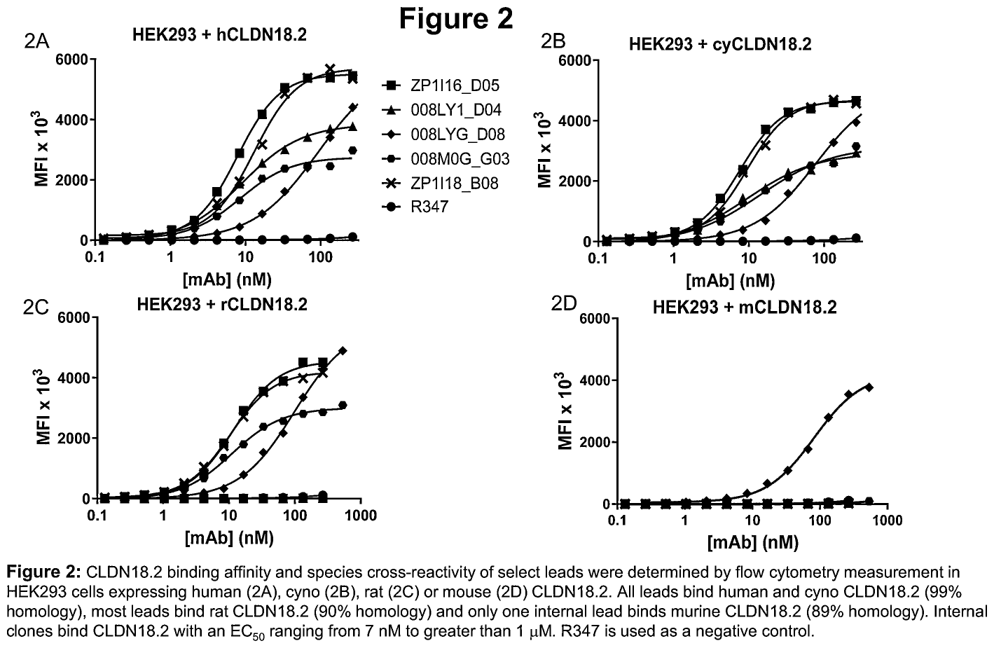

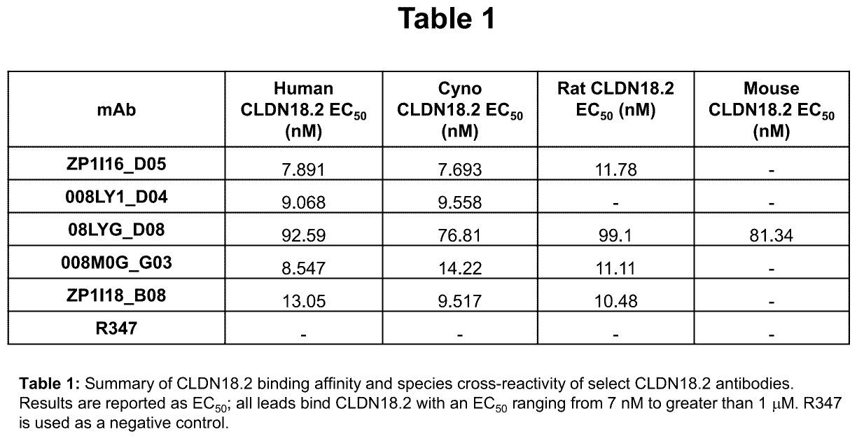

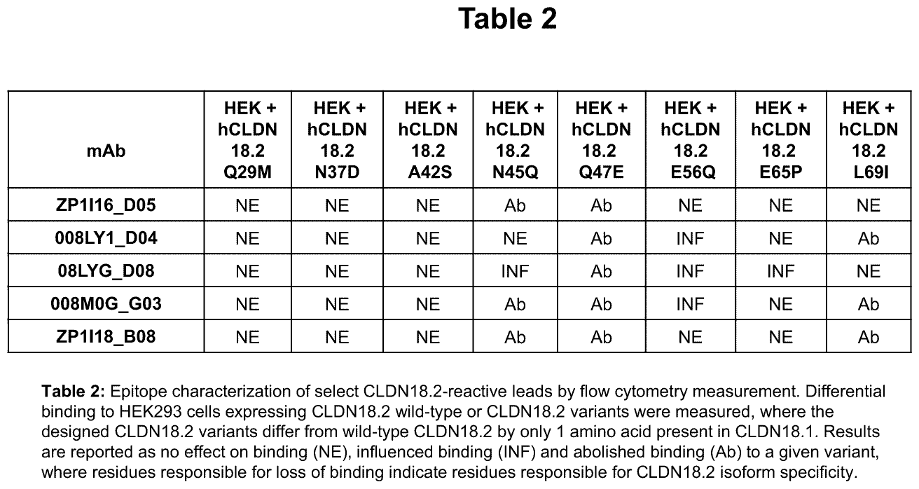

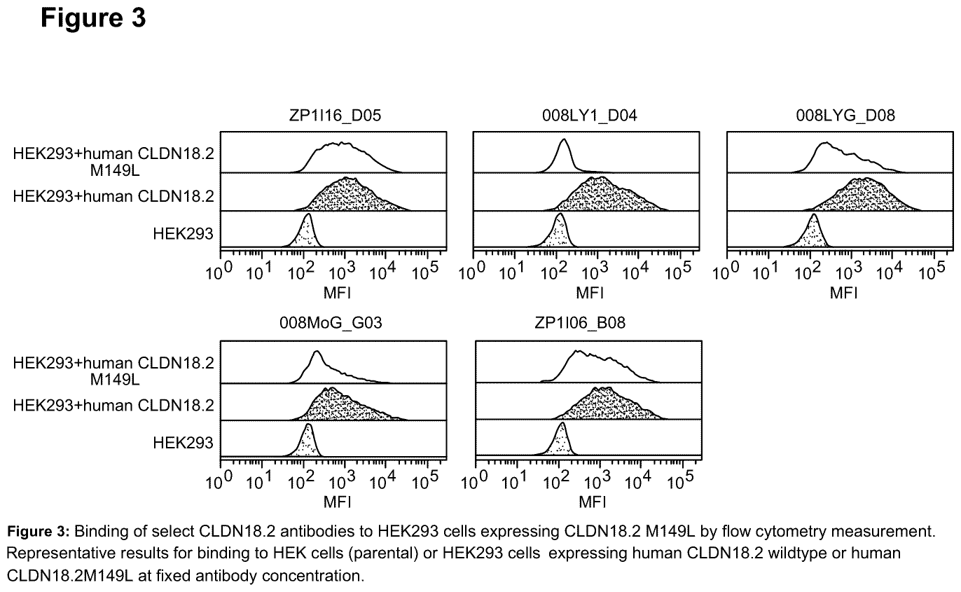

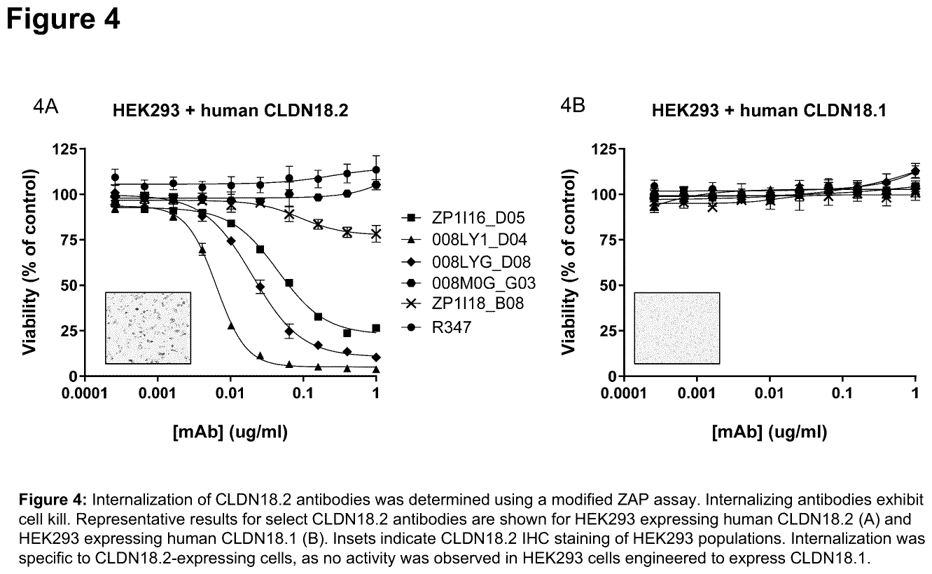

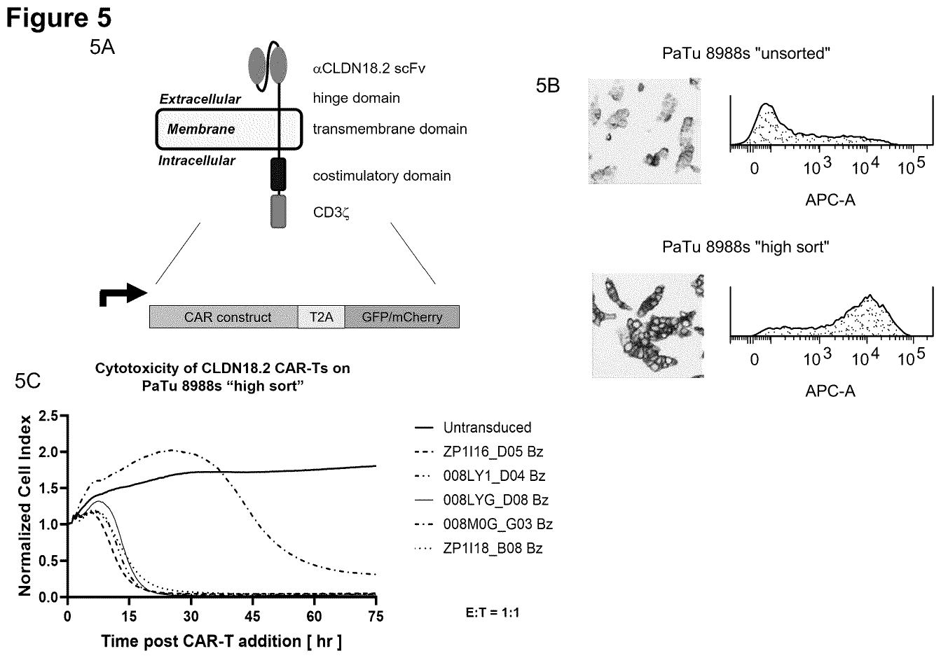

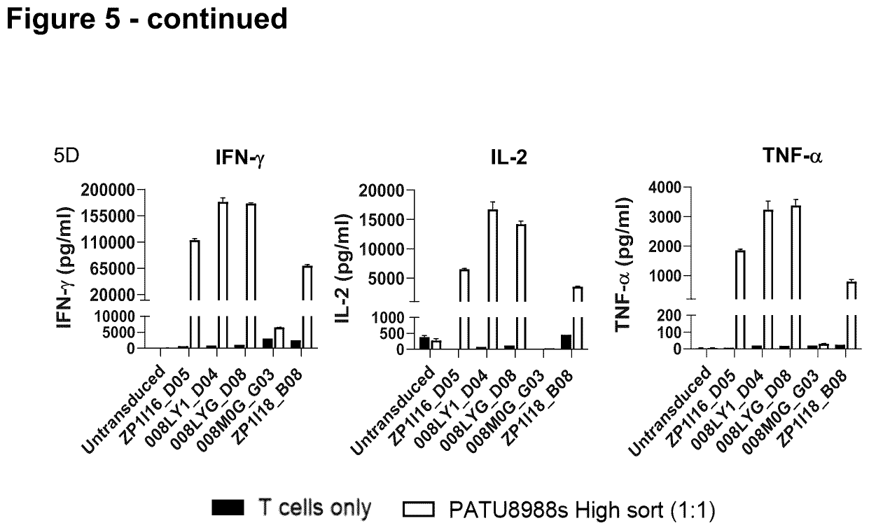

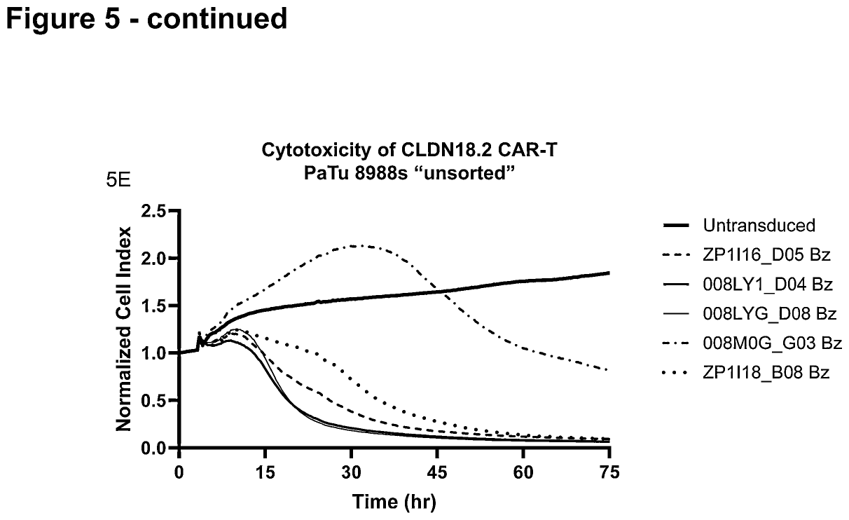

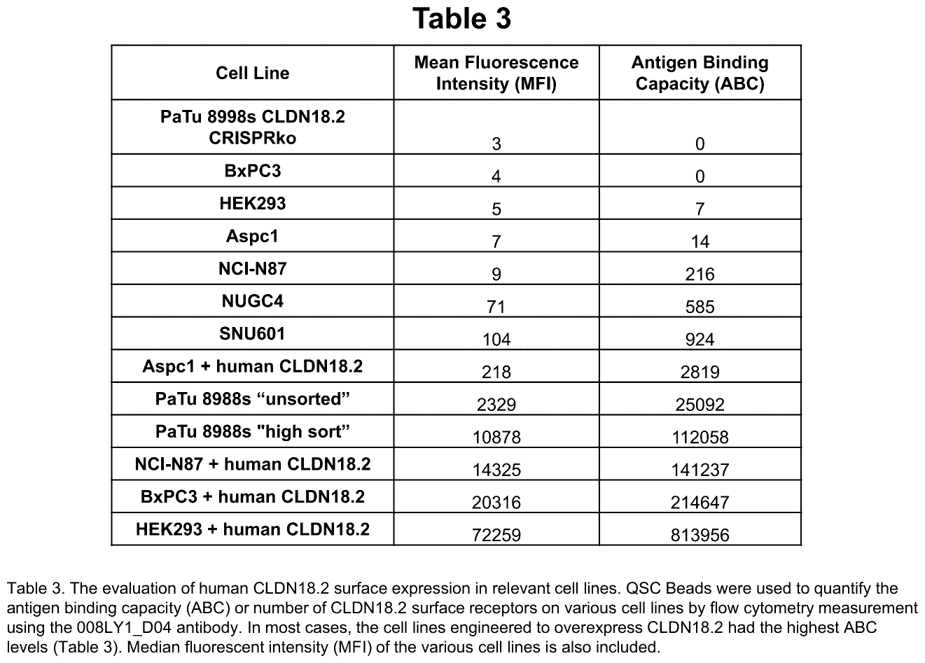

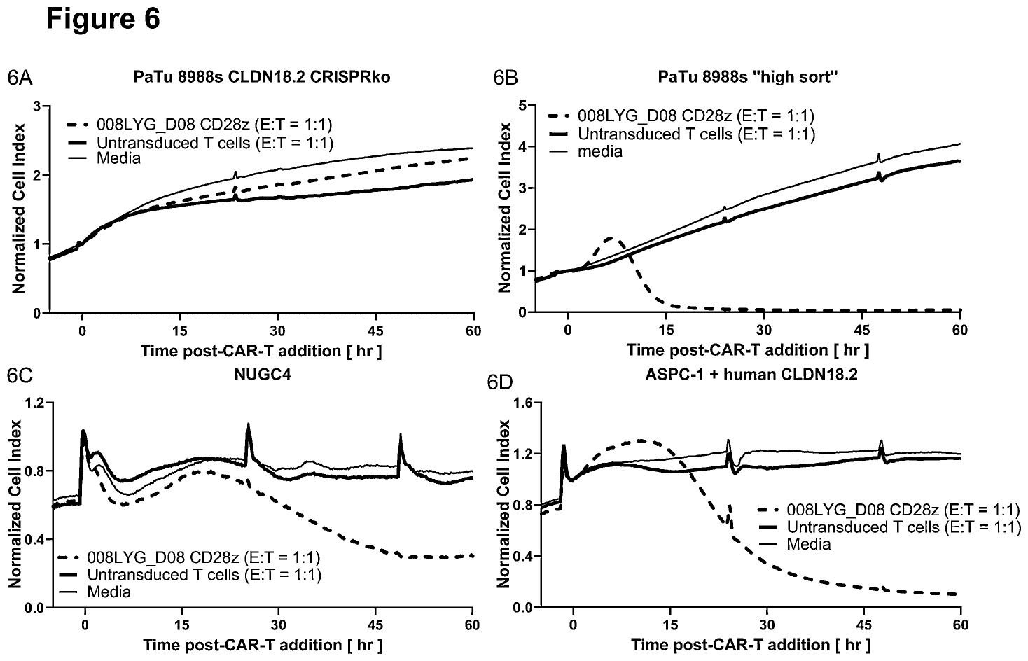

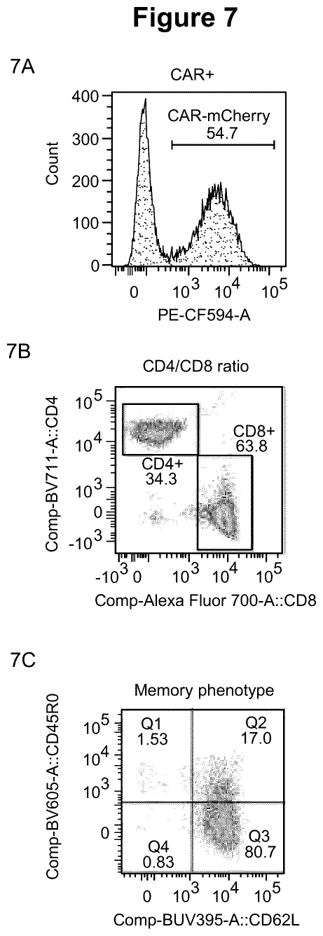

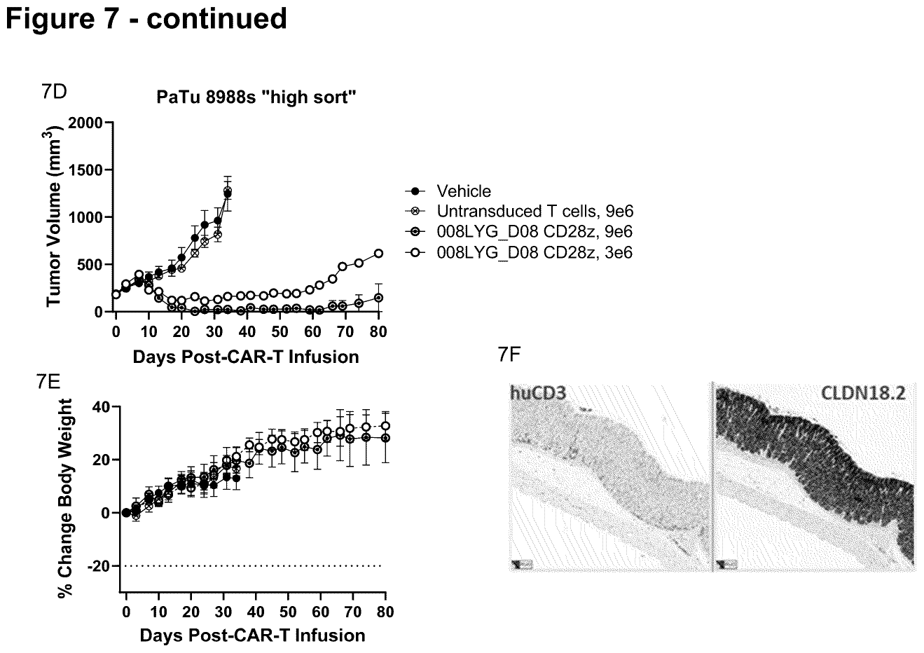

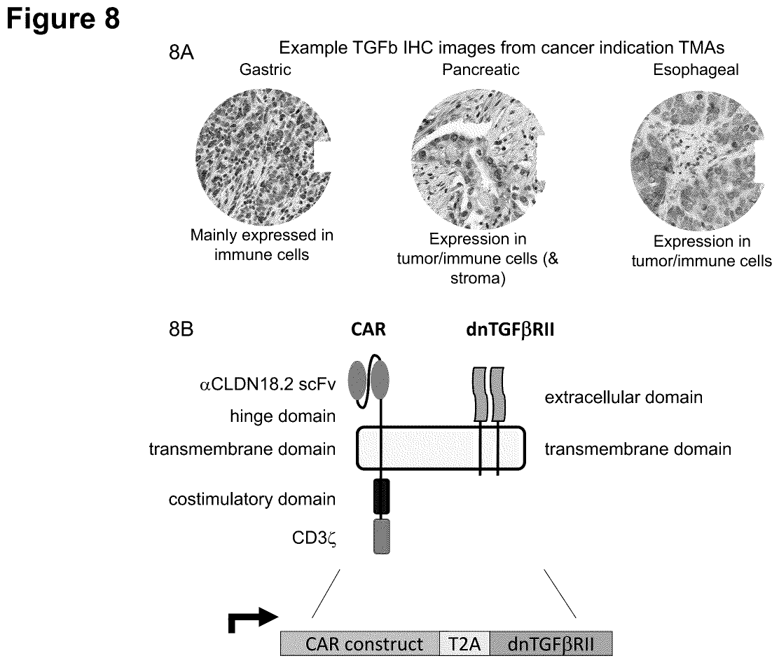

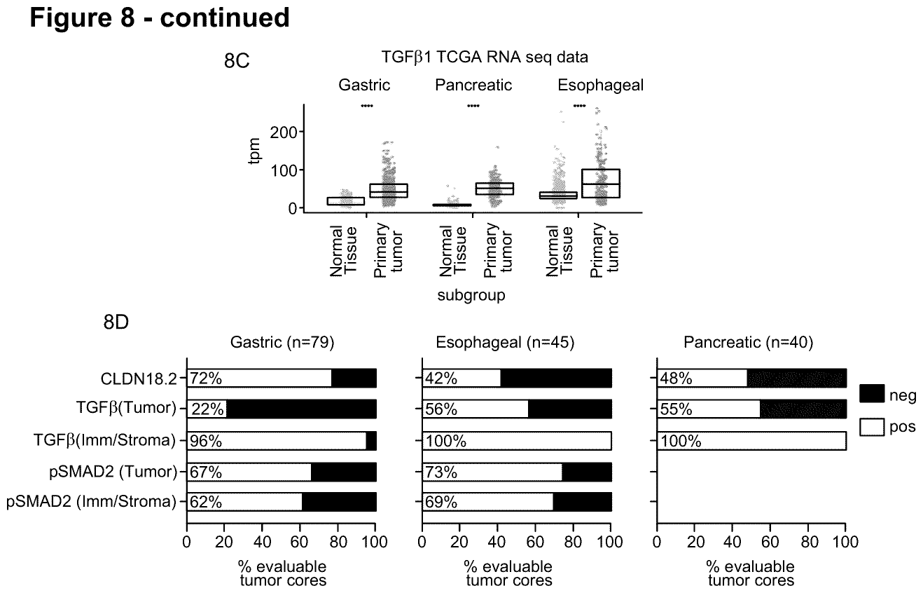

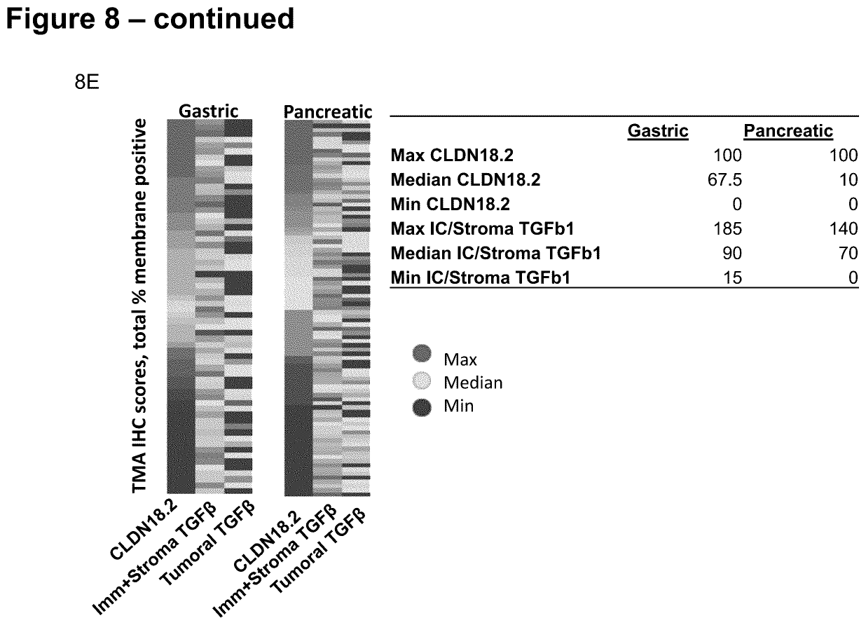

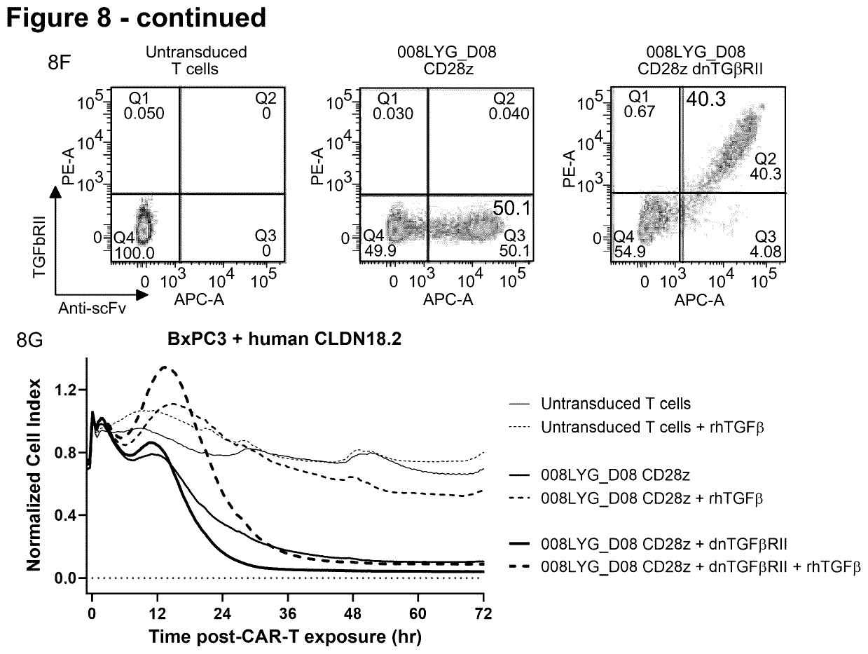

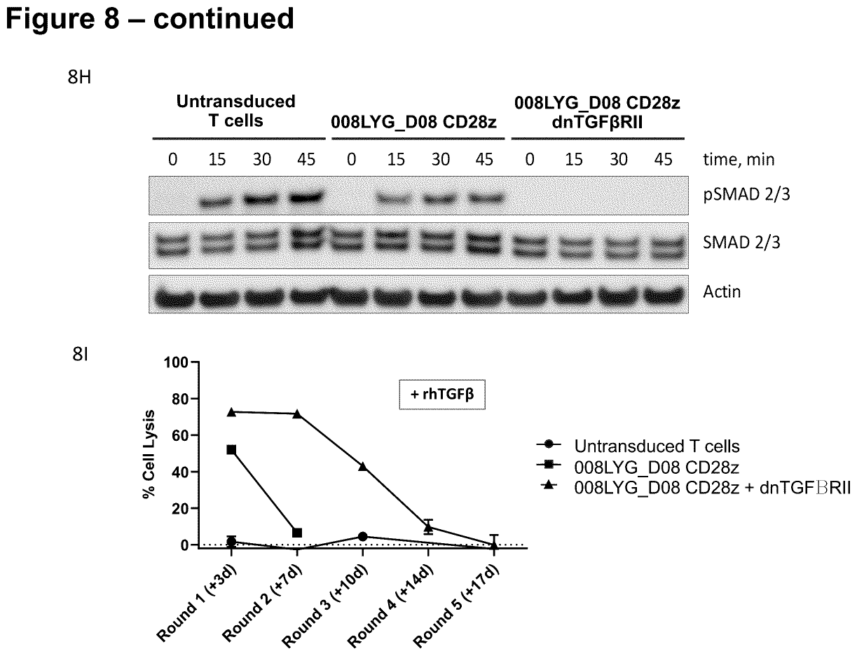

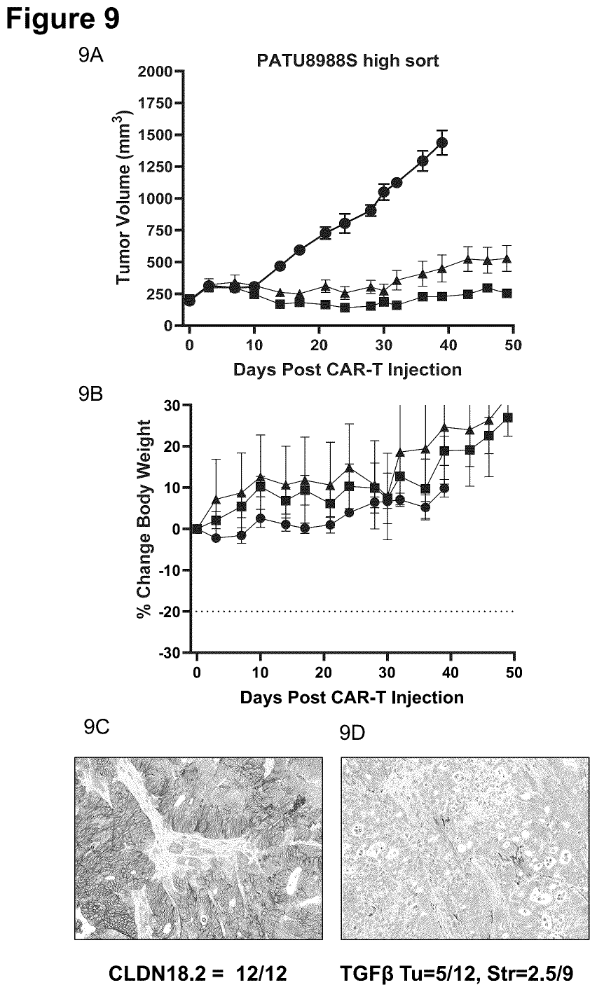

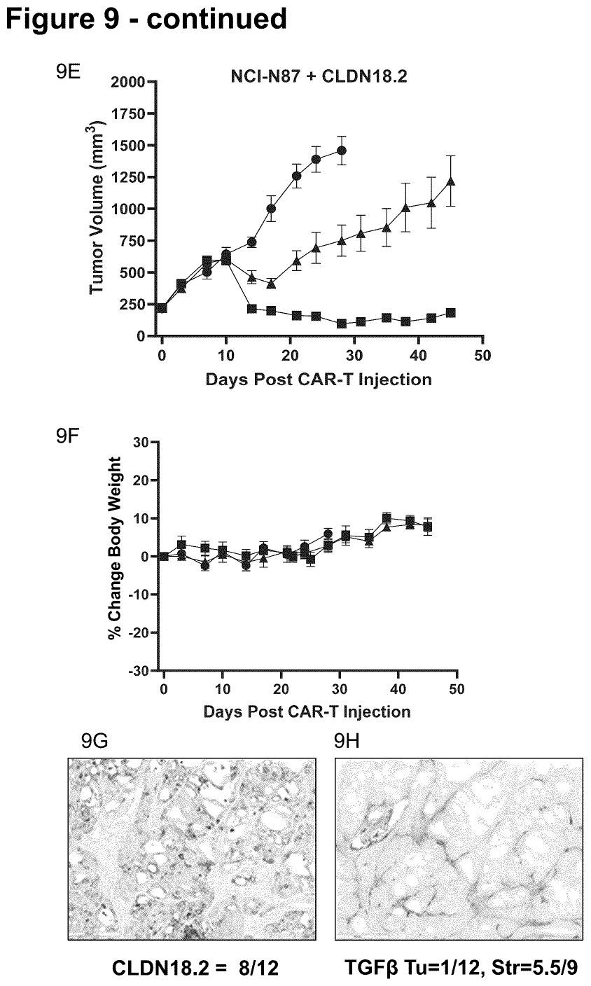

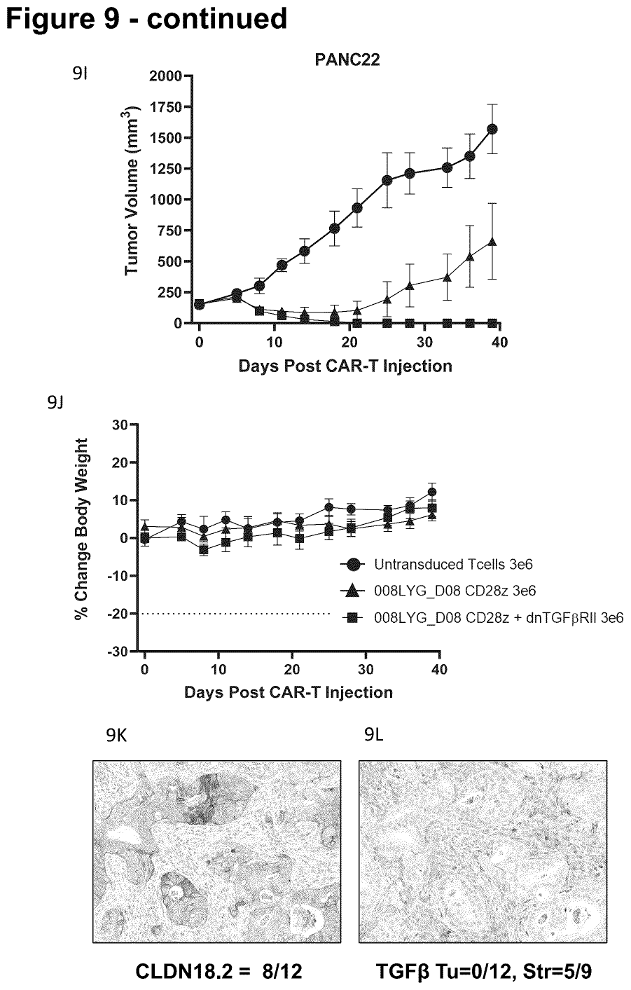

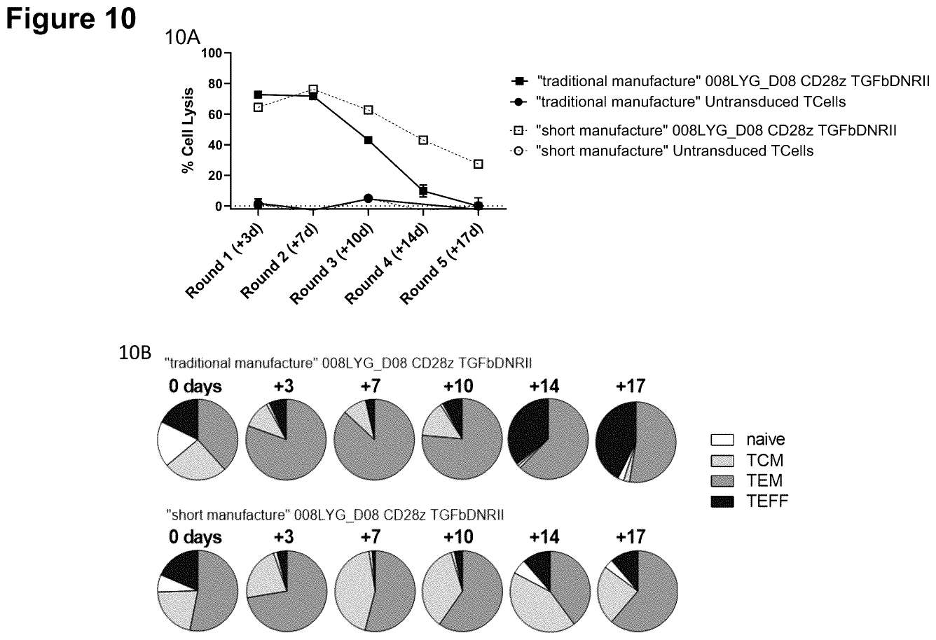

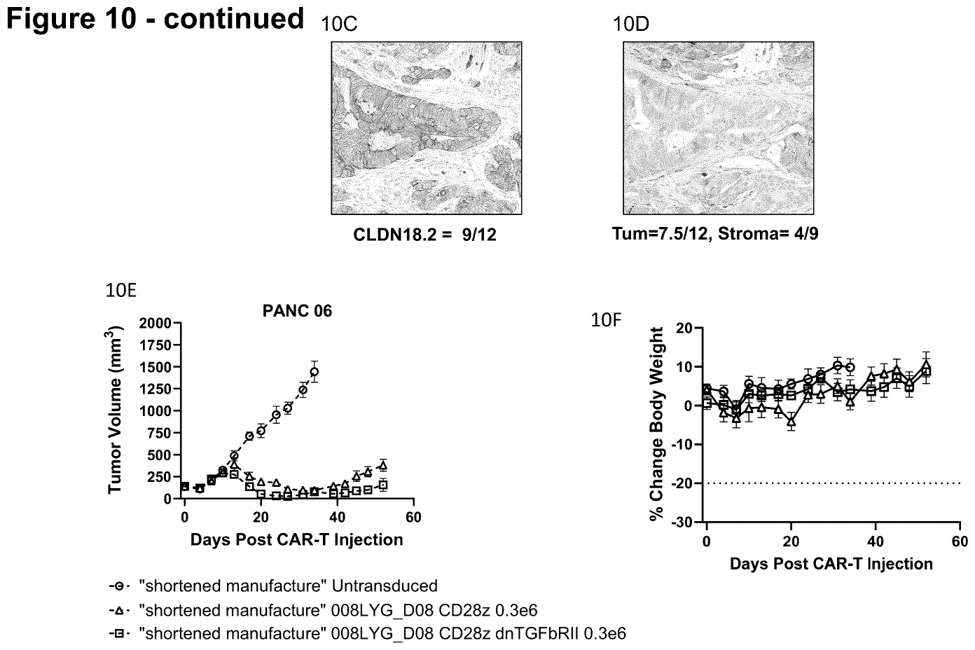

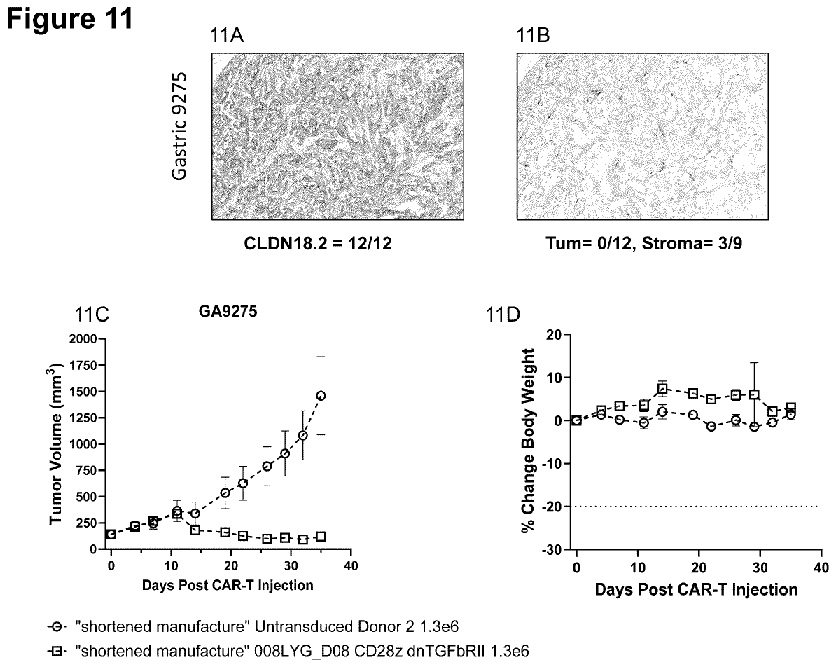

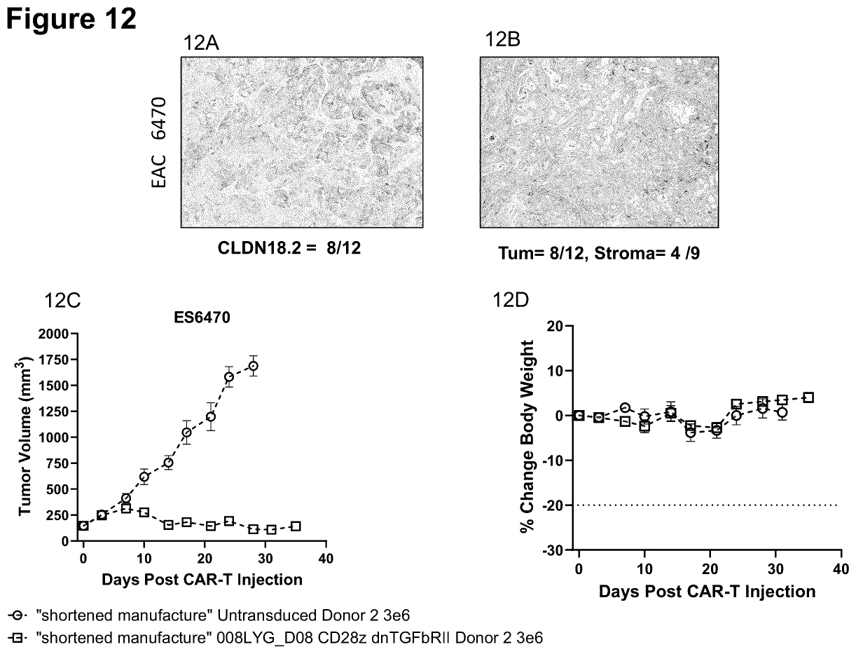

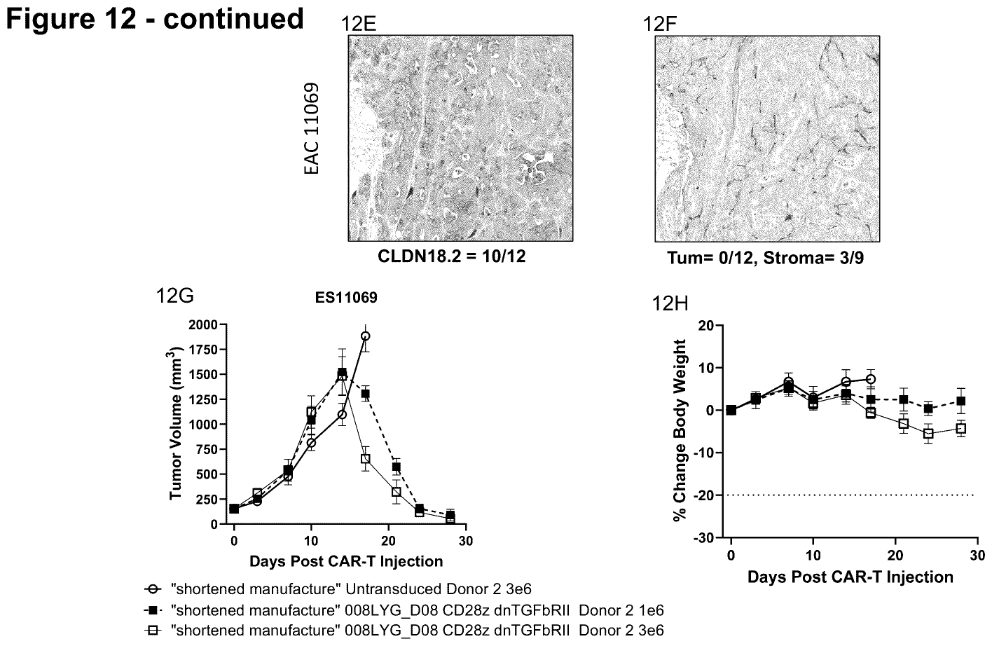

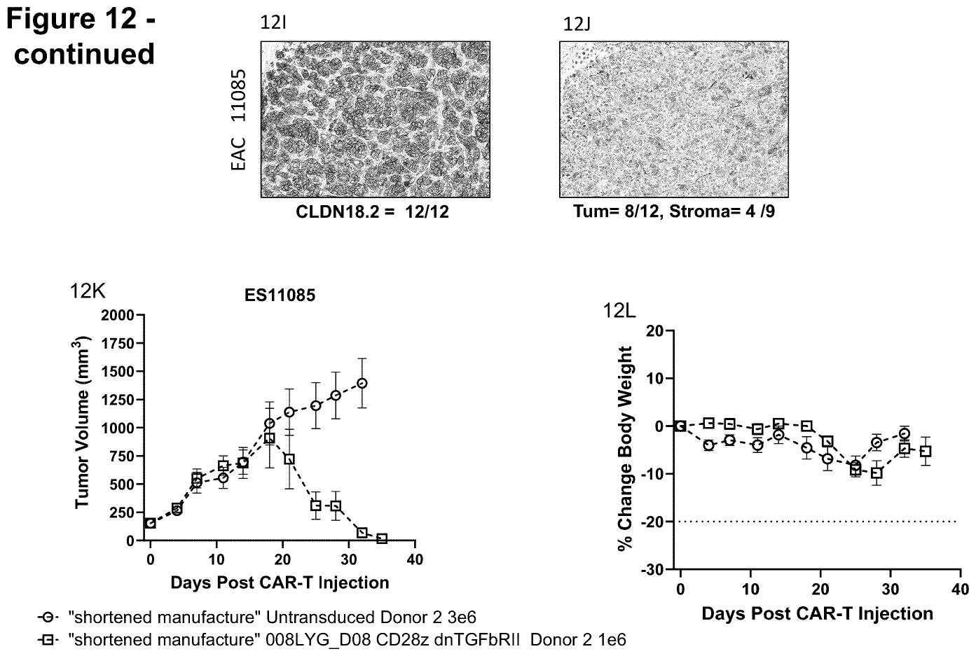

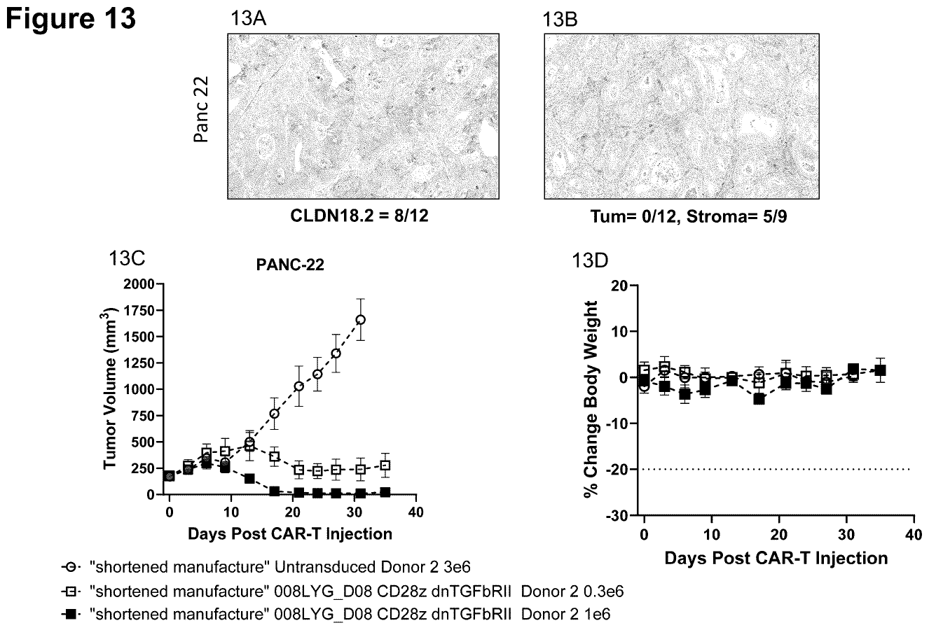

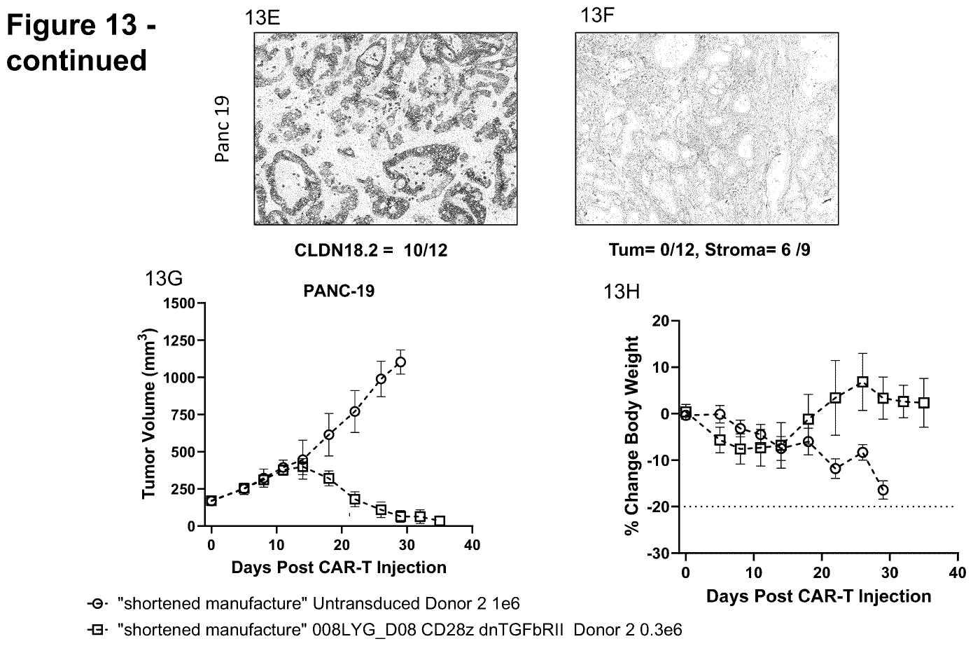

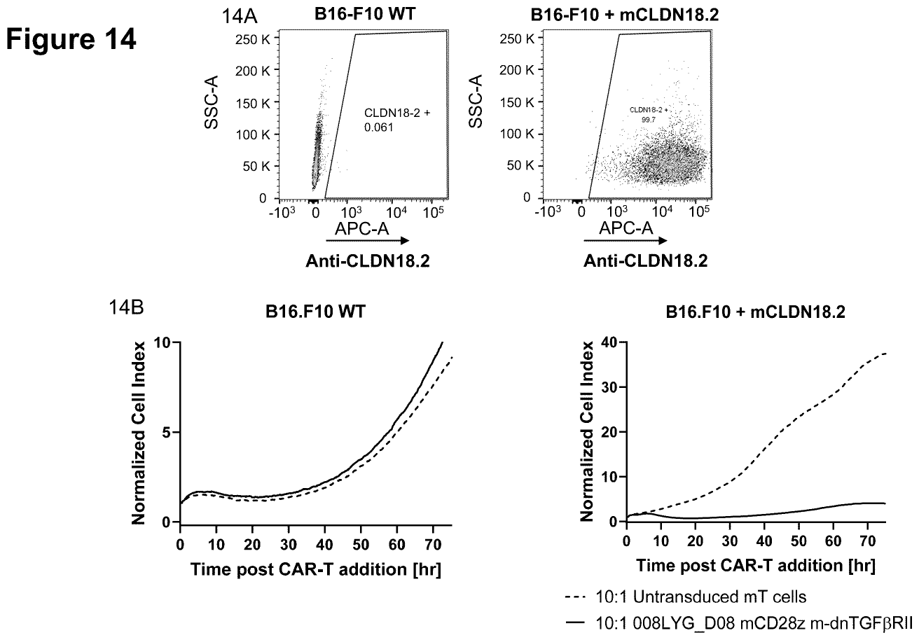

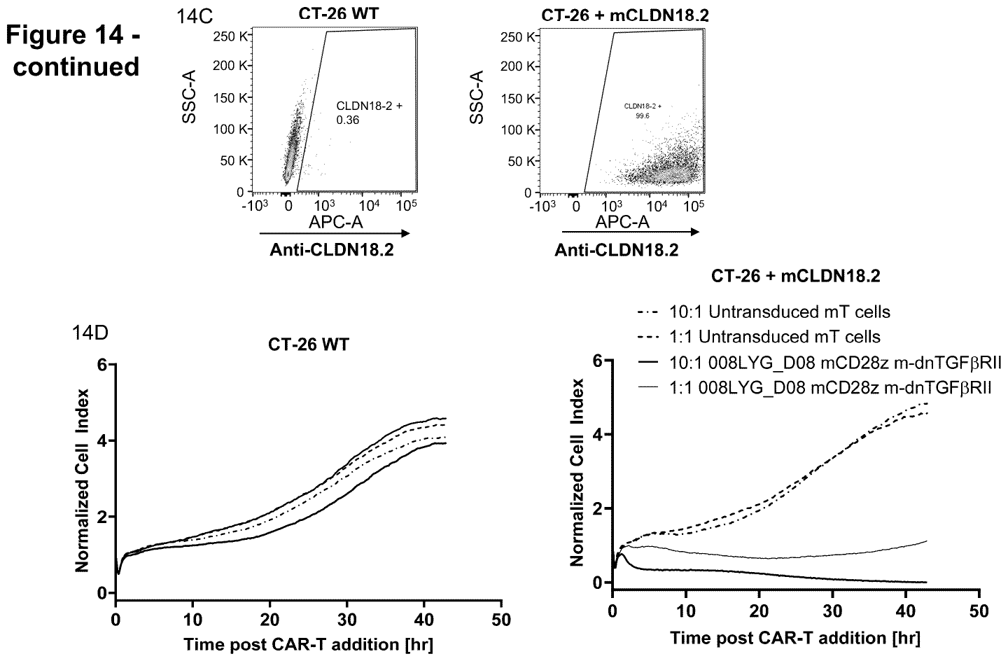

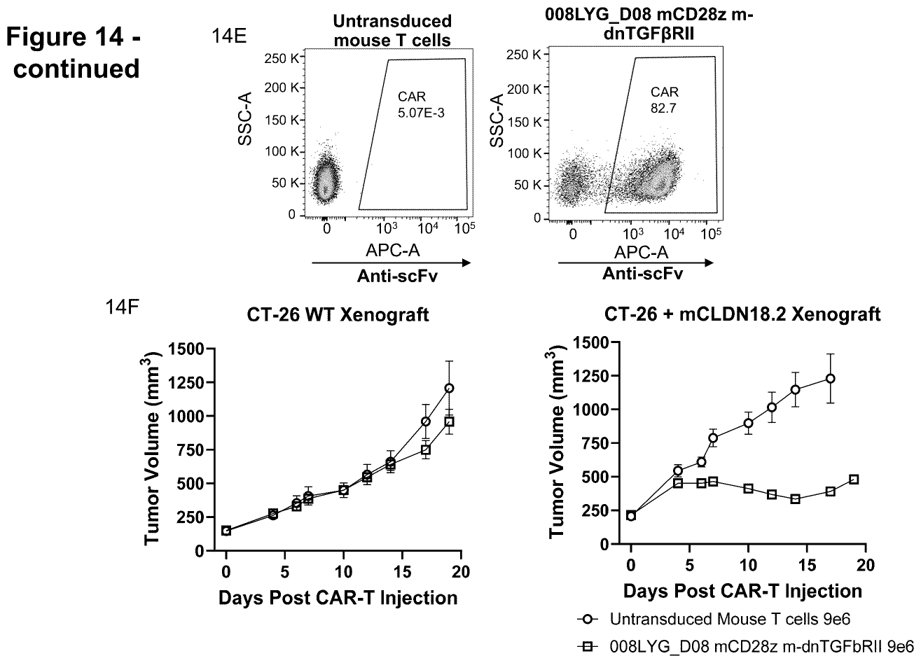

Table 1 provides a summary of CLDN18.2 binding affinity and species cross-reactivity of select CLDN18.2 antibodies. Results are reported as EC₅₀; all leads bind CLDN18.2 with an EC₅₀ ranging from 7 nM to greater than 1 μM. R347 is used as a negative control.

Table 2 summarizes the epitope characterization of select CLDN18.2-reactive leads by flow cytometry measurement. Differential binding to HEK293 cells expressing CLDN18.2 wild-type or CLDN18.2 variants were measured, where the designed CLDN18.2 variants differ from wild-type CLDN18.2 by only 1 amino acid present in CLDN18.1. Results are reported as no effect on binding (NE), influenced binding (INF) and abolished binding (Ab) to a given variant, where residues responsible for loss of binding indicate residues responsible for CLDN18.2 isoform specificity.

FIG. 3 shows binding histograms of select CLDN18.2 antibodies to HEK293 cells expressing CLDN18.2 M149L as measured by flow cytometry measurement. Representative results for binding to HEK cells (parental), HEK293 cells expressing human CLDN18.2 wildtype or human CLDN18.2 M149L at a fixed antibody concentration are shown.

FIGS. 4A-4B show the internalization properties of select CLDN18.2 antibodies as determined using a modified ZAP assay. Internalizing antibodies exhibit cell kill. Representative results for select CLDN18.2 antibodies are shown for HEK293 expressing human CLDN18.2 (FIG. 4A) and HEK293 expressing human CLDN18.1 (FIG. 4B). Insets indicate CLDN18.2 IHC staining of HEK293 populations. Internalization was specific to CLDN18.2-expressing cells, as no activity was observed in HEK293 cells engineered to express CLDN18.1. R347 is a negative control antibody.

FIGS. 5A-5E show a representative CAR-T design and data. FIG. 5A: CAR-T design. The design of the CAR-T is shown in FIG. 5A, with the CAR construct containing the CLDN18.2 binding domain, hinge domain, transmembrane domain, costimulatory domain and CD3z signaling domain, a self-cleaving peptide, and GFP/mCherry for ease of detection. FIG. 5B: IHC images of PaTu 8988s cell lines used in the assay. Representative IHC images of the “unsorted” and “high sort” PaTu 8988s cell pellets as well as flow cytometry plots with 008LY1_D04 are shown (FIG. 5B). FIG. 5C: Cell lysis capacity of various clones via in vitro xCELLigence assay using PaTu 8988s “high sort”. CAR-T tumor cell killing capacity was measured using the Agilent xCELLigence Real-Time Cell Analysis system. Tumor cell lysis is monitored as the normalized cell index drops to 0 on the x-axis. At a fixed ratio of 1 CAR-T cell to 1 tumor cell (E:T=1:1), clones ZP1I16_D05, 008LY1_D04, 008LYG_D08 and ZP1I18_B08 showed equivalent and potent cell lytic capacity reaching ˜50% at 15 h post-CAR-T introduction, whereas clone 008M0G_G03 CAR-Ts exhibited delayed cell lysis, reaching ˜50% at 60 h post-CAR-T introduction. FIG. 5D: Cytokine measurements of supernatant pulled from assay at 24 hours. 24 hours post-addition of CAR-T cells to the well, 25 ul of the culture medium was sampled to examine for T cell secreted pro-inflammatory cytokines using an MSD ELISA assay following the manufacturer's instructions. 008LY1_D04 and 008LYG_D08 had the largest and relatively equivalent cytokine secretion levels for IFN-γ, IL-2 and TNF-α, with reduced levels present for ZP1I16_D05 and ZP1I18_B08. Minimal IFNγ production was detected for clone 008M0G_G03. For all conditions, wells without cancer cells but just T cells alone in the cancer cell media were used as controls. Despite a heterogeneous expression, similar trends in potency of the various clones as CAR-Ts were observed in the PaTu 8988s “unsorted” cell line, with 008LY1_D04 and 008LYG_D08 remaining the most potent. FIG. 5E: Cell lysis capacity of various clones via in vitro xCELLigence assay using PaTu 8988s “unsorted” cells.

Table 3 shows the evaluation of human CLDN18.2 surface expression in relevant cell lines. QSC Beads were used to quantify the antigen binding capacity (ABC) or number of CLDN18.2 surface receptors on various cell lines by flow cytometry measurement using the 008LY1_D04 antibody. In most cases, the cell lines engineered to overexpress CLDN18.2 had the highest ABC levels (Table 3). Median fluorescent intensity (MFI) of the various cell lines is also included.

FIGS. 6A-6D shows the correlation of CLDN18.2 density to 008LYG_D08 CD28z CAR-T-mediated cell lytic capacity using the Agilent xCELLigence Real-Time Cell Analysis system. In each assessment, equivalent E:T ratios were maintained for CAR-T cells and untransduced matched donor T cells. Each cancer cell line was plated in an eSight 96 well plate at a pre-determined density for each cell line (40-50,000 cells per well). Media alone serves as a negative control and lack residual activity of the untransduced T cells. PaTu 8988s with CRISPR knock out of CLDN18.2 and an ABC=0 (FIG. 6A) were compared to the activity in PaTu 8988s “high sort” with an ABC=112,058 per cell (FIG. 6B). Maximal cell lysis was achieved by 15 hours post-CAR-T cell addition with the PaTu 8988s “high sort” cells, with no cell lysis observed in the CLDN18.2 knockout line up to the assay endpoint, which demonstrates specificity of 008LYG_D08 CD28z CAR-T cell lysis for cells that express CLDN18.2. NUGC4 gastric cancer cells with a low receptor density (ABC=585 per cell, FIG. 6C) and AsPC1 engineered to express a moderate CLDN18.2 receptor density (ABC=2819 per cell, FIG. 6D) were also examined. 008LYG_D08 CD28z CAR-T were able to lyse these cancer cells with low and moderate antigen expression patterns, though the kinetics and max lysis varied.

FIGS. 7A-7F show in vitro characterization of transduced CAR-T cells and in vivo efficacy and safety profiles of NSG mice dosed with 008LYG_D08 CD28z CAR-Ts. Using a murine cross-reactive CAR-T enabled simultaneous measurement of tumor lytic capacity and early assessment of potential safety concerns. CAR-T cells were generated with purified total T cells as described in materials and methods. CAR-T cells were expanded for 12 days prior to infusion, with CAR-T cell media exchanged every two days. CAR+ expression was determined via assessing mCherry fluorescence and determined to be 55% (FIG. 7A). The percentage of these CAR+ cells that were CD4 or CD8 was also determined (FIG. 7B), where a slightly higher percentage of CD8+ (64%) over CD4+ (34%) CAR-T cells were observed. Memory phenotype was determined pre-infusion (FIG. 7C) by examining CD45RO by CD62L expression, which showed 008LYG_D08 CD28z CAR-T maintained a less differentiated, CD62L⁺/CD45RO⁻ phenotype. In vivo efficacy of 008LYG_D08 CD28z CAR-T were carried out in 6 to 8-week-old female NOD.Cg-PrkdcscidIl2rgtm1Wj1/SzJ (Jackson Laboratories, NSG) mice. On day −10, 10E6 PaTu 8988s “high sort” cells were implanted subcutaneously in Cultrex™ Basement Membrane Extract (BME), Type 3 in the upper right flank. On day 0, when xenografts reached an average size of 180 to 200 mm³, mice were dosed intravenously with a single infusion of 3E6 or 9E6 008LYG_D08 CD28z CAR-T cells, 9E6 untransduced donor matched T cells, or vehicle control. 9E6 008LYG_D08 CD28z CAR-T led to potent tumor xenograft regression out to 65 days in a dose-dependent manner (FIG. 7D), with no associated body weight loss (FIG. 7E). 3E6 008LYG_D08 CD28z CAR-T caused tumor stasis with eventual outgrowth beyond day 60. At the study endpoint, tissues from the normal stomach from a representative animal were evaluated by IHC; staining for huCD3 indicates T cell infiltration and staining CLDN18.2 indicates normal stomach expression (FIG. 5F). These staining patterns show that CLDN18.2 gastric expression is maintained and is consistent with control animals. Overall, this study demonstrated a dose-dependent and long-lasting tumor response with no body weight loss indicative of a potential safety window.

FIGS. 8A-8I show rationale for dnTGFβRII armoring and proof of mechanism. Representative IHC images stained with TGFβ1 are shown for gastric, pancreatic and esophageal adenocarcinoma samples, with one image showing a sample with only immune cells stained positive and another showing all three components positive for TGFβ (FIG. 8A). Inhibitory cytokines can also lead to recruitment of suppressor cells such as Tregs and MDSCs. TGFβ is known to induce T cell exhaustion through downstream signaling of the receptor complex and limit CAR-T persistence and lytic capacity. To boost efficacy of CAR-Ts in the solid tumor setting, one approach that can be employed is use of a dominant negative TGFβ receptor II. The CAR-T design (FIG. 8B) is shown using a T2A self-cleaving peptide which enables expression of both the CAR and the dominant negative receptor in the T cells driven from a single promoter. CAR-T cells were generated with purified total T cells as described in materials and methods. CLDN18.2 CAR-Ts armored with the dominant negative TGFβRII may possess an advantage in gastric, pancreatic and esophageal cancers which all present with elevated TGFβ1 RNA expression in primary tumors as compared to normal tissue as per The Cancer Genome Atlas Program (TCGA) database (National Cancer Institute) (FIG. 8C). Data from 3 individual and indication-specific multispot tumor microarrays (TMAs) were profiled for CLDN18.2, TGFβ1 and phospho SMAD2 (gastric and esophageal only) by IHC. IHC expression was scored for positivity at 1% for any intensity of tumor or immune/stromal compartments. In gastric, esophageal and pancreatic cancer indications, TGFβ1 stain was observed in 22%, 56% and 55% of samples, respectively; in all indications TGFβ1 was abundantly observed in the immune and stromal cells at positivity of 1% of cells at any intensity (FIG. 8D). Phospho SMAD2 was also examined as a measure of TGFβ pathway activation, in the gastric and esophageal TMAs. Abundant staining for phospho SMAD2 was observed and correlated with TGFβ1 IHC, which indicates that TGFβ was active in these tumor samples. To further support selection of armoring, additional gastric and pancreatic TMAs were IHC stained for CLDN18.2 and TGFβ1 to cover a larger number of patient samples and IHC score was converted to a heat map of maximal and minimal as well as median level of CLDN18.2 or TGFβ1 expression (FIG. 8E). TGFβ1 was observed at varied levels in the tumor cells and immune and stromal cells, and varied across CLDN18.2 expression. The abundance and expression of TGFβ in all cells of the TME will increase likelihood of success of the armoring approach. Taken together, these data support dnTGFβRII armoring as a rational approach to overcome the tumor microenvironment and promote CAR-T activity. Prior to use in downstream assays, surface CAR+ and dnTGFβRII were detected by flow cytometry (FIG. 8F). Untransduced cells were negative for both markers, 008LYG_D08 CD28z were 50.1% CAR+ by the anti-scFv and 008LYG_D08 CD28z dnTGFβRII were 40.3% dual positive by both markers. To demonstrate in vitro proof of mechanism of the dominant negative TGFβRII armoring, cell lysis was measured using an xCELLigence assay as described in materials and methods. BXPC3 (pancreatic adenocarcinoma) cells were engineered to overexpress human CLDN18.2. These cells were co-cultured with untransduced donor-matched T cells, 008LYG_D08 CD28z CAR-T or 008LYG_D08 CD28z dnTGFβRII armored CAR-T in the absence or presence of 10 ng/ml recombinant human TGFβ at a constant E:T ratio and a matched total T cell number per well in the 96 well plate. In media alone, both CAR-Ts efficiently lysed CLDN18.2-expressing cells, with similar potency but slightly earlier max cell lysis were observed with the dnTGFβRII-armored CAR-Ts. In contrast, culture medium supplemented with 10 ng/ml recombinant human TGFβ reduced cell lytic capacity of the unarmored CAR-T but cell lysis was maintained for the dominant negative TGFβRII-expressing CAR-Ts (FIG. 8G). Additional proof of mechanism of the dominant negative TGFβRII was examined by monitoring of downstream signaling capacity via western blot. Induction of downstream phospho SMAD-2/3 signaling as early as 15 minutes post-co-incubation with recombinant human TGFβ was observed for the matched donor untransduced T cells and 008LYG_D08 CD28z, however, no signaling was observed up to 45 minutes after TGFβ exposure for the 008LYG_D08 CD28z dnTGFβRII CAR-Ts. Total SMAD-2/3 and loading control actin protein levels were consistent for all groups (FIG. 8H). Lastly, to demonstrate the advantages of CAR-T cells with mechanisms to overcome TGFβ suppression, a serial antigen restimulation assay was carried out. At each time point, the same CAR-T cells were added at a fixed E:T ratio (1:2) to BXPC3 cells were engineered to overexpress CLDN18.2. The assay was carried out with addition of 10 ng/ml recombinant human TGFβ to the culture medium. These data demonstrate that the armored CAR-Ts cells maintained a greater level of cell lysis and could do so for multiple rounds beyond unarmored CAR-Ts in the presence of TGFβ (FIG. 8I).

FIGS. 9A-9L show in vivo proof of mechanism of dominant negative TGFβRII armoring. Examination of in vivo efficacy of 008LYG_D08 CD28z and 008LYG_D08 CD28z dnTGFβRII armored CAR-T was carried out in 6 to 8-week-old female NSG MHC I/II KO mice. CAR-T cells were generated with purified total T cells as described in materials and methods. Cell line xenograft models PaTu 8988s (“high sort”, pancreatic) and NCI-N87 (gastric) engineered to overexpress CLDN18.2 as well as a patient-derived pancreatic xenograft model were implanted in Cultrex™ Basement Membrane Extract (BME), Type 3 and grown until tumors reached an average of 150-200 mm³. Animals were then dosed intravenously with a single infusion of 3E6 untransduced donor matched T cells, unarmored CAR-T or dnTGFβRII-armored CAR-T cells, all groups matched for an equivalent total T cell infusion within each experimental study. Long lasting in vivo efficacy was observed for both 008LYG_D08 CD28z CAR-T and 008LYG_D08 CD28z dnTGFβRII armored CAR-T cells in the PaTu 8988s model (FIG. 9A) with no body weight loss observed (FIG. 9B). Representative IHC images of CLDN18.2-specific staining (FIG. 9C) and TGFβ1 staining (FIG. 9D) are shown. This model was the highest in CLDN18.2 expression examined, with a score of 12/12, and this may reflect the equivalent activity of the two CAR-T constructs irrespective of TGFβ IHC scores. This model had a TGFβ tumor cell stain score of 5/12 and stromal cell compartment stain score of 2.5/9. Long lasting and superior in vivo efficacy was observed more specifically for the 008LYG_D08 CD28z dnTGFβRII armored CAR-T cells in the NCI-N87 engineered to overexpress CLDN18.2 model at a CAR-T single infusion dose of 3e6 cells (FIG. 9E) with no body weight loss observed (FIG. 9F). Representative IHC images of CLDN18.2-specific staining (FIG. 9G) and TGFβ1 staining (FIG. 9H) are shown. This model was scored as an 8/12 for CLDN18.2 expression, and with a TGFβ tumor cell stain score of 1/12 and stromal cell compartment stain score of 5.5/9. Similarly, long lasting and superior in vivo efficacy was observed following a single infusion of the 008LYG_D08 CD28z dnTGFβRII armored CAR-T cells in the Panc22 PDX model with 5/5 animals exhibiting a complete response at the study endpoint whereas early response to day 20 but subsequent tumor regrowth was observed in the 008LYG_D08 CD28z CAR-T group (FIG. 9I) with no body weight loss observed (FIG. 9J). Representative IHC images of CLDN18.2-specific staining (FIG. 9K) and TGFβ1 staining (FIG. 9L) are shown. This PDX model was scored as an 8/12 in CLDN18.2 expression, and with a TGFβ tumor cell stain score of 0/12 and stromal cell compartment stain score of 5/9.

FIGS. 10A-10F show effects of manufacturing on CAR-T persistence and memory phenotype. 008LYG_D08 CD28z dnTGFβRII armored CAR-T cells generated using the “traditional manufacture” process compared to cells generated with the “shortened manufacture” process were compared side-by-side in the serial antigen stimulation assay, co-cultured with BxPC3 cells engineered to overexpress CLDN18.2 at an E:T ratio of 1:2. Early in the assay both groups proved to have similar efficacy in tumor cell lysis, but on repeated antigen stimulation, “traditional manufacture” cells had lysed less than 10% of cancer cells in the fourth round of antigen stimulation, whereas the “shortened manufacture” cells still maintained lysis of 43% at the same time point (FIG. 10A). At each time point during the serial antigen restimulation assay, CAR+ T cells from each group were phenotyped for memory status using CD45RO by CD62L expression, with CD62L⁺/CD45RO⁻ representing naïve or less differentiated cells (T_{N}), CD62L⁺/CD45RO⁺ representing central memory cells (T_{CM}), CD62L⁻/CD45RO⁺ representing effector memory cells (T_{EM}) and CD62L⁻/CD45RO⁻ representing the most terminally differentiated (T_{EFF}) subset of the group. While both manufacturing processes generated cell populations on day 0 with relatively similar phenotype, the “traditional manufacture” cells had a larger percentage of T_{EM} cells at day 7 and 10, and this translated to a larger percentage of terminally differentiated T_{EFF} at day 14 and 17, demonstrating the impact of manufacture protocol on cell functionality, persistent tumor cell lysis and phenotype (FIG. 10B). Long-lasting in vivo efficacy was observed with “short manufacture” or SMART 008LYG_D08 CD28z and 008LYG_D08 CD28z dnTGFβRII armored CAR-T cells in the Panc06 PDX model at a CAR-T single infusion dose of 0.3E6 cells (FIG. 10E) with no body weight loss observed (FIG. 10F). Representative IHC images of CLDN18.2-specific staining (FIG. 10C) and TGFβ1 staining (FIG. 10D) are shown. This model was scored as an 9/12 in CLDN18.2 expression, and with a TGFβ tumor cell stain score of 7.5/12 and stromal cell compartment stain score of 4/9.

FIGS. 11A-11D show efficacy data in a xenograph model of gastric cancer treated with 008LYG_D08 CD28z dnTGFβRII armored CAR-T cells from a second T cell donor in a PDX model of gastric cancer (FIG. 11C), with long lasting tumor control and no body weight loss at the 1.3e6 dose (FIG. 11D). Representative IHC images of CLDN18.2-specific staining (FIG. 11A, scored as a 12/12) and TGFβ1 staining (FIG. 11B, scored as a tumor 0/12 and stroma 3/9) for this gastric cancer PDX are shown.

FIGS. 12A-12L show efficacy data in xenograph models of esophageal adenocarcinoma treated with 008LYG_D08 CD28z dnTGFβRII armored CAR-T cells from a second T cell donor in the ES6470 PDX model of esophageal adenocarcinoma (FIGS. 12A-D), the ES11069 PDX model of esophageal adenocarcinoma (FIGS. 12 E-H), and the ES11085 PDX model of esophageal adenocarcinoma (FIGS. 12 I-L) with evidence of tumor control (FIG. 12C, 12G, and 12K) and no significant body weight loss at the various doses shown (FIG. 12D, 12H, and 12L). Representative IHC images of CLDN18.2-specific staining (FIG. 12A, scored as a 8/12, FIG. 12E, scored as a 10/12 and FIG. 12I, scored as a 12/12) and TGFβ1 staining (FIG. 12B, FIG. 12F and FIG. 12J) for the selected esophageal adenocarcinoma PDXs are shown.

FIGS. 13A-13H show efficacy data in xenograph models of pancreatic cancer treated with 008LYG_D08 CD28z dnTGFβRII armored CAR-T cells from a second T cell donor in PDX models of pancreatic cancer Panc22 (FIGS. 13A-D) and Panc19 (FIGS. 13E-H), with evidence of tumor control (FIG. 13C, 13G) and no significant body weight loss at the various doses shown (FIG. 13D, 13H). Representative IHC images of CLDN18.2-specific staining (FIG. 13A, scored as a 8/12, FIG. 13E, scored as a 10/12) and TGFβ1 staining (FIG. 13B and FIG. 13F) for the selected pancreatic adenocarcinoma PDXs are shown.

FIGS. 14A-14F show efficacy of 008LYG_DO8 CAR-T cells in syngeneic murine models of melanoma (B16-F10) and colon carcinoma (CT-26) engineered to express murine CLDN18.2. The engineered B16-F10+mCLDN18.2 cells were 99.7% positive for CLDN18.2 compared to B16-F10 WT cells (FIG. 14A). The CT-26+mCLDN18.2 cells were 99.6% positive for CLDN18.2 compared to CT-26 WT cells (FIG. 14C). In vitro cell lysis capacity of 008LYG_DO8 mCD28z m-dnTGFbRII via in vitro xCELLigence assay showed potent lysis specifically of cells which express CLDN18.2 using B16-F10 cells and CAR-T at a 10:1 E:T ratio (FIG. 14B) and CT-26 cells at 10:1 and 1:1 ratios (FIG. 14D). A single murine CAR-T infusion led to tumor growth inhibition specifically in CLDN18.2 expressing xenografts (FIG. 14F), with no significant body weight loss (data not shown).

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present disclosure.

### DETAILED DESCRIPTION

Unless defined otherwise, all technical and scientific terms used herein have the meaning commonly understood by a person skilled in the art to which this disclosure belongs. The following references provide one of skill with a general definition of many of the terms used in this disclosure: Singleton, et al., Dictionary of Microbiology and Molecular Biology (2nd ed. 1994); The Cambridge Dictionary of Science and Technology (Walker ed., 1988); The Glossary of Genetics, 5th Ed., R. Rieger, et al. (eds.), Springer Verlag (1991); and Hale & Marham, The Harper Collins Dictionary of Biology (1991). As used herein, the following terms have the meanings ascribed to them below, unless specified otherwise.

As used herein, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indicates otherwise.

Percentages disclosed herein can vary in amount by ±10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. The term “about” also includes the exact amount. For example, “about 5%” means “about 5%” and also “5%.” The term “about” can also refer to ±10% of a given value or range of values. Therefore, about 5% also means 4.5%-5.5%, for example. Additionally, “about” or “comprising essentially of” can mean a range of up to ±10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition. Unless otherwise clear from context, all numerical values provided herein are modified by the term “about.”

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

As used herein, the terms “or” and “and/or” can describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

As used herein, the term “polypeptide” refers to a molecule composed of monomers (amino acids) linearly linked by amide bonds (also known as peptide bonds). The term “polypeptide” refers to any chain or chains of two or more amino acids. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms.

A “protein” as used herein can refer to a single polypeptide, i.e., a single amino acid chain as defined above, but can also refer to two or more polypeptides that are associated, e.g., by disulfide bonds, hydrogen bonds, or hydrophobic interactions, to produce a multimeric protein.

An “isolated” substance, e.g., isolated nucleic acid, is a substance that is not in its natural milieu, though it is not necessarily purified. For example, an isolated nucleic acid is a nucleic acid that is not produced or situated in its native or natural environment, such as a cell. An isolated substance can have been separated, fractionated, or at least partially purified by any suitable technique.

As used herein, the terms “antibody” and “antigen-binding fragment thereof” refer to at least the minimal portion of an antibody which is capable of binding to a specified antigen which the antibody targets, e.g., at least some of the complementarity determining regions (CDRs) of the variable domain of a heavy chain (VH) and the variable domain of a light chain (VL) in the context of a typical antibody produced by a B cell. In some antibodies, e.g., naturally-occurring IgG antibodies, the heavy chain constant region is comprised of a hinge and three domains, CH1, CH2 and CH3. In some antibodies, e.g., naturally-occurring IgG antibodies, each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. A heavy chain can have the C-terminal lysine or not. Unless specified otherwise herein, the amino acids in the variable regions are numbered using the Kabat numbering system and those in the constant regions are numbered using the EU system.

Antibodies or antigen-binding fragments thereof can be or be derived from polyclonal, monoclonal, human, humanized, or chimeric antibodies, single chain antibodies, epitope-binding fragments, e.g., Fab, Fab′ and F(ab′)2, Fd, Fv, single-chain fragment variable (scFv), single-chain antibodies, VHH, vNAR, nanobody, (single-domain antibody), disulfide-linked Fvs (sdFvs), fragments comprising either a VL or VH domain alone or in conjunction with a portion of the opposite domain (e.g., a whole VL domain and a partial VH domain with one, two, or three CDRs), and fragments produced by a Fab expression library. ScFv molecules are known in the art and are described, e.g., in U.S. Pat. No. 5,892,019. Antibody molecules encompassed by this disclosure can be of or be derived from any type (e.g., IgG, IgE, IgM, IgD, IgA, and IgY), class (e.g., IgG1, IgG2, IgG3, IgG4, IgA1 and IgA2), or subclass of immunoglobulin molecule.

As used herein, antibodies or antigen-binding fragments thereof also include “single-domain antibodies” which are antibodies whose complementary determining regions are part of a single domain polypeptide. Examples of single domain antibodies include heavy chain antibodies, antibodies naturally devoid of light chains, single domain antibodies derived from conventional four-chain antibodies, engineered or recombinant single-domain antibodies. Single domain antibodies may be derived from any species including, but not limited to mouse, human, camel, llama, goat, rabbit, and bovine. Single domain antibodies may be naturally occurring single domain antibodies known as heavy chain antibody devoid of light chains. In particular, Camelidae species, for example camel, dromedary, llama, alpaca and guanaco, produce heavy chain antibodies naturally devoid of light chain. The variable heavy chain of single-domain antibodies devoid of light chains are known as “VHH” or “nanobody”. Similar to conventional VH domains, VHHs contain four FRs and three CDRs. Nanobodies have advantages over conventional antibodies: they are smaller than IgG molecules, and as a consequence properly folded functional nanobodies can be produced by in vitro expression while achieving high yield. For example, VHH domains, Nanobodies and proteins/polypeptides containing the same can be produced using microbial fermentation and do not require the use of mammalian expression systems; VHH domains and nanobodies are relatively small (approximately 15 kDa, or 10 times smaller than a conventional IgG), and therefore show high(er) penetration into tissues (including but not limited to solid tumors and other dense tissues) than such conventional 4-chain antibodies and antigen-binding fragments thereof; VHH domains and nanobodies can show so-called cavity-binding properties (inter alia due to their extended CDR3 loop, compared to conventional VH domains) and can therefore also access targets and epitopes not accessible to conventional 4-chain antibodies and antigen-binding fragments thereof. Furthermore, nanobodies are very stable, and resistant to the action of proteases.

As used herein, “VHH domain” refers to variable domains present in naturally occurring heavy-chain antibodies, in order to distinguish them from the heavy chain variable domains that are present in conventional four-chain antibodies (referred to herein as “VH domains”) and from the light chain variable domains that present in conventional four-chain antibodies (referred to herein as “VL domains”). In some embodiments, the recombinant polypeptides of the disclosure correspond to amino acid sequences of naturally occurring VHH domains, but that have been “humanized,” i.e., by replacing one or more amino acid residues in the amino acid sequence of the naturally occurring VHH sequence by one or more of the amino acid residues that occur at the corresponding positions in a VH domain from a conventional four-chain antibody from a human being. This can be performed in a manner known in the art.

In one embodiment, the disclosure provides recombinant polypeptide sequences, such as immunoglobulin sequences (in some embodiments, VHH antibody sequences) that are capable of binding to an envelope epitope of CLDN18.2, wherein the immunoglobulin sequence comprises four framework regions (FR1, FR2, FR3, and FR4) and three complementarity determining regions (CDR1, CDR2, and CDR3), wherein:
a) CDR1 is the amino acid sequence of SEQ ID NO:1, 11, 21, 31, or 41; or selected from the group consisting of amino acid sequences that have at least 85%, or at least 90%, or at least 95%, or at least 99% sequence identity with the amino acid sequence of SEQ ID NO: 1, 11, 21, 31, or 41; or from the group consisting of amino acid sequences that have 2 or only 1 amino acid differences as compared to the amino acid sequence of SEQ ID No: 1, 11, 21, 31, or 41;
b) CDR2 is the amino acid sequence of SEQ ID NO:2, 12, 22, 32, or 42; or selected from the group consisting of amino acid sequences that have at least 85%, or at least 90%, or at least 95%, or at least 99% sequence identity with the amino acid sequence of SEQ ID NO: 2, 12, 22, 32, or 42; or from the group consisting of amino acid sequences that have 2 or only 1 amino acid differences as compared to the amino acid sequence of SEQ ID No: 2, 12, 22, 32, or 42;
c) CDR3 is the amino acid sequence of SEQ ID NO:3, 13, 23, 33, or 43; or selected from the group consisting of amino acid sequences that have at least 85%, or at least 90%, or at least 95%, or at least 99% sequence identity with the amino acid sequence of SEQ ID NO:3, 13, 23, 33, or 43; or from the group consisting of amino acid sequences that have 2 or only 1 amino acid differences as compared to the amino acid sequence of SEQ ID No:3, 13, 23, 33, or 43;
   and in which the framework sequences may be any suitable framework sequences, such as the framework sequences of a single-domain antibody and in particular of a VHH antibody.

The term “antigen-binding portion” of an antibody, as used herein, refers to one or more fragments of an antibody that retain the ability to specifically bind to an antigen (e.g., human CLDN18.2). The antigen-binding function of an antibody can be performed by fragments of a full-length antibody. Examples of binding fragments encompassed within the term “antigen-binding portion” of an antibody, e.g., an anti-CLDN18.2 antibody described herein, include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “chimeric antigen receptor” or “CAR,” as used herein, refers to an engineered antigen-binding polypeptide, comprising an antigen-binding domain, a transmembrane domain, and one or more intracellular domains (e.g. costimulatory domains). In some embodiments, a CAR can optionally comprise a spacer domain and/or a flexible hinge domain to provide conformational freedom to facilitate binding to the target antigen on the target cell. In some embodiments, a CAR can optionally comprise an armoring domain comprising a nucleic acid sequence encoding an armoring molecule. Expression of a CAR on the surface of a cell, e.g., an immune cell, allows the cell to target and bind a particular antigen. In some embodiments, the CAR is expressed by an immune cell, e.g., a T cell. In some embodiments, the antigen binding domain comprises an Fab, Fab′, F(ab′)2, Fd, Fv, single-chain fragment variable (scFv), single chain antibody, VHH, vNAR, nanobody (single-domain antibody), or any combination thereof. In some embodiments, the transmembrane domain comprises a transmembrane domain selected from the transmembrane domain of CD4, CD8α, or CD28. In some embodiments, the one or more intracellular domains comprises a costimulatory domain or a portion thereof. In some embodiments, the intracellular domain comprises a costimulatory domain or a portion thereof. In some embodiments, the intracellular domain comprises a costimulatory domain of CD3z or variants thereof. For instance, the CD3z costimulatory domain variants may contain only 1 or 2 functional immunoreceptor tyrosine-based activation motifs (ITAMs) of the three ITAMs present in wild-type CD3z. In some embodiments, the intracellular domain comprises a costimulatory domain selected from the group consisting a CD3zeta costimulatory domain, CD28 costimulatory domain, a CD27 costimulatory domain, a 4-1BB costimulatory domain, an ICOS costimulatory domain, an OX-40 costimulatory domain, a GITR costimulatory domain, a CD2 costimulatory domain, an IL-2Rβ costimulatory domain, a MyD88/CD40 costimulatory domain, and any combination thereof. A CAR can further comprise a “hinge” or “spacer” domain. Non-limiting examples of hinge/spacer domains include immunoglobulin hinge/spacer domains, such as an IgG1 hinge domain, and IgG2 hinge domain, an IgG3 hinge domain, an IgG4 hinge domain, an IgG4P hinge domain (an IgG4 hinge domain comprising a S241P mutation), or a CD8a hinge domain, or a CD28 hinge domain.

As used herein, the term “polynucleotide” includes a singular nucleic acid as well as multiple nucleic acids, and refers to an isolated nucleic acid molecule or construct, e.g., messenger RNA (mRNA) or plasmid DNA (pDNA). The term “nucleic acid” includes any nucleic acid type, such as DNA or RNA. “Conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In some embodiments, a predicted nonessential amino acid residue in a CLDN18.2-binding moiety (e.g., an anti-CLDN18.2 CAR or antibody) is replaced with another amino acid residue from the same side chain family.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (freely available), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and)(BLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules described herein. BLAST protein searches can be performed with the)(BLAST program, score=50, word length=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g.,)(BLAST and NBLAST) can be used.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., lentiviral vectors, replication defective retroviruses, adenoviruses and adeno-associated viruses), or transposons (e.g. DNA transposons or retrotransposons) which serve equivalent functions. In certain embodiments, the CARs and/or antibodies or antigen binding fragments thereof are encompassed by and/or delivered to a cell and or patient using a virus, a lentivirus, an adenovirus, a retrovirus, an adeno-associated virus (AAV), a transposon, a DNA vector, a mRNA, a lipid nanoparticle (LNP), or a CRISPR-Cas System. In an embodiment, lentiviral vectors are used.

As used herein, the term “vector” can refer to a nucleic acid molecule as introduced into a host cell, thereby producing a transformed host cell. A vector can include nucleic acid sequences that permits it to replicate in a host cell, such as an origin of replication. A vector can also include one or more selectable marker gene(s) and other genetic elements known in the art. Specific types of vector envisioned here can be associated with or incorporated into viruses to facilitate cell transformation.

A “transformed” cell, or a “host” cell, is a cell into which a nucleic acid molecule has been introduced by molecular biology techniques. All techniques by which a nucleic acid molecule can be introduced into such a cell, including transfection with viral vectors, transformation with plasmid vectors, and introduction of naked DNA by electroporation, lipofection, and particle gun acceleration are contemplated herein. In certain embodiments, cells are transformed by one or more techniques using a virus, a lentivirus, an adenovirus, a retrovirus, an adeno-associated virus (AAV), a transposon, a DNA vector, a mRNA, a lipid nanoparticle (LNP), and a CRISPR-Cas System.

As used herein, the term “affinity” refers to a measure of the strength of the binding of a antigen or target (such as an epitope) to its cognate binding domain (such as a paratope). As used herein, the term “avidity” refers to the overall stability of the complex between a population of epitopes and paratopes (i.e., antigens and antigen binding domains).

The term “epitope” refers to a site on an antigen (e.g., CLDN18.2) to which a chimeric antigen receptor, immunoglobulin, or antibody specifically binds, e.g., as defined by the specific method used to identify it. Epitopes can be formed both from contiguous amino acids (usually a linear epitope) or non-contiguous amino acids juxtaposed by tertiary folding of a protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation.

“Immunotherapy” refers to the treatment of a subject afflicted with, or at risk of contracting or suffering a recurrence of, a disease by a method comprising inducing, enhancing, suppressing or otherwise modifying the immune system or an immune response. An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell.

As used herein, the terms “treat,” “treatment,” or “treatment of” when used in the context of treating cancer refer to reducing disease pathology, reducing or eliminating disease symptoms, promoting increased survival rates, and/or reducing discomfort. For example, treating can refer to the ability of a therapy when administered to a subject, to reduce disease symptoms, signs, or causes. Treating also refers to mitigating or decreasing at least one clinical symptom and/or inhibition or delay in the progression of the condition and/or prevention or delay of the onset of a disease or illness.

As used herein, the terms “subject,” “individual,” or “patient,” refer to any subject, particularly a mammalian subject, for whom diagnosis, prognosis, or therapy is desired. Mammalian subjects include, for example, humans, non-human primates, dogs, cats, guinea pigs, rabbits, rats, mice, horses, cattle, bears, and so on.

As used herein, the term an “effective amount” or a “therapeutically effective amount” of an administered therapeutic substance, such as a CAR T cell, is an amount sufficient to carry out a specifically stated or intended purpose, such as treating or treatment of cancer. An “effective amount” can be determined empirically in a routine manner in relation to the stated purpose.

The terms “T cell” or “T lymphocyte” are art-recognized and are intended to include thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), a tumor infiltrating cytotoxic T cell (TIL; CD8⁺ T cell), CD4⁺CD8⁺ T cell, CD4⁻CD8⁻ T cell, or any other subset of T cells. Other illustrative populations of T cells suitable for use in particular embodiments include naive T cells and memory T cells.

As used herein, the term “proliferation” refers to an increase in cell division, either symmetric or asymmetric division of cells. In particular embodiments, “proliferation” refers to the symmetric or asymmetric division of T cells. “Increased proliferation” occurs when there is an increase in the number of cells in a treated sample compared to cells in a non-treated sample.

The term “expanding” in the method of the disclosure refers to the process of increasing the number of cells in a cell culture. In the expanding step, cells are fed and culture media is replaced at regular intervals, in one embodiment according to a feed regimen. The specific timings and amounts of media added in a particular feed regimen will depend on the cell number and the levels of metabolites in the culture.

As used herein, the term “differentiation” refers to a method of decreasing the potency or proliferation of a cell or moving the cell to a more developmentally restricted state. In particular embodiments, differentiated T cells acquire immune effector cell functions.

An “immune effector cell,” is any cell of the immune system that has one or more effector functions (e.g., cytotoxic cell killing activity, secretion of cytokines, induction of ADCC and/or CDC). The illustrative immune effector cells contemplated herein are T lymphocytes, in particular cytotoxic T cells (CTLs; CD8⁺ T cells), TILs, and helper T cells (HTLs; CD4⁺ T cells).

“Modified T cells” refer to T cells that have been modified by the introduction of a polynucleotide encoding an engineered CAR contemplated herein. Modified T cells include both genetic and non-genetic modifications (e.g., episomal or extrachromosomal).

As used herein, the term “genetically engineered” or “genetically modified” refers to the addition of extra genetic material in the form of DNA or RNA into the total genetic material in a cell.

The terms, “genetically modified cells,” “modified cells,” and, “redirected cells,” are used interchangeably.

The acronym “SMART” (Shorty-Manipulated Auto-Replicating T-Cells) refers to a shortened T cell manufacture and expansion process wherein the cells are cultured in the presence of IL-21 (and optionally IL-2).

The acronym “TNT” (Traditionally Nurtured T-Cells) refers to a traditional T-cell expansion process which does not employ IL-21, and typically comprises a cell culture for more than 7 days and/or typically comprises the use of IL-2.

The term “stimulation” refers to a primary response induced by binding of a stimulatory molecule (e.g., a TCR/CD3 complex) with its cognate ligand thereby mediating a signal transduction event including, but not limited to, signal transduction via the TCR/CD3 complex.

A “stimulatory molecule,” refers to a molecule on a T cell that specifically binds with a cognate stimulatory ligand.

A “stimulatory ligand,” as used herein, means a ligand that when present on an antigen presenting cell (e.g., an APC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands include, but are not limited to CD3 ligands, e.g., an anti-CD3 antibody and CD2 ligands, e.g., anti-CD2 antibody, and peptides, e.g., CMV, HPV, EBV peptides.

The term, “activation” refers to the state of a T cell that has been sufficiently stimulated to induce detectable cellular proliferation. In particular embodiments, activation can also be associated with induced cytokine production, and detectable effector functions. The term “activated T cells” refers to, among other things, T cells that are proliferating. Signals generated through the TCR alone are insufficient for full activation of the T cell and one or more secondary or costimulatory signals are also required. Thus, T cell activation comprises a primary stimulation signal through the TCR/CD3 complex and one or more secondary costimulatory signals. Costimulation can be evidenced by proliferation and/or cytokine production by T cells that have received a primary activation signal, such as stimulation through the CD3/TCR complex or through CD2.

A “costimulatory signal,” refers to a signal, which in combination with a primary signal, such as TCR/CD3 ligation, leads to T cell proliferation, cytokine production, and/or upregulation or downregulation of particular molecules (e.g., CD28).

A “costimulatory ligand,” refers to a molecule that binds a costimulatory molecule. A costimulatory ligand may be soluble or provided on a surface. A “costimulatory molecule” refers to the cognate binding partner on a T cell that specifically binds with a costimulatory ligand (e.g., anti-CD28 antibody).

“Autologous,” as used herein, refers to cells from the same subject. In some embodiments, the cells of the disclosure are autologous.

“Allogeneic,” as used herein, refers to cells of the same species that differ genetically to the cell in comparison. In some embodiments, the cells of the disclosure are allogeneic.

“Syngeneic,” as used herein, refers to cells of a different subject that are genetically identical to the cell in comparison. In some embodiments, the cells of the disclosure are syngeneic.

“Xenogeneic,” as used herein, refers to cells of a different species to the cell in comparison. In some embodiments, the cells of the disclosure are xenogeneic.

As used herein, the terms “individual” and “subject” are often used interchangeably and refer to any animal that exhibits a symptom of a cancer that can be treated with the gene therapy vectors, cell-based therapeutics, and methods disclosed elsewhere herein. Suitable subjects (e.g., patients) include laboratory animals (such as mouse, rat, rabbit, or guinea pig), farm animals, and domestic animals or pets (such as a cat or dog). Non-human primates and, preferably, human patients, are included. Typical subjects include human patients that have a cancer, have been diagnosed with a cancer, or are at risk or having a cancer.

By “enhance” or “promote,” or “increase” or “expand” refers generally to the ability of a composition contemplated herein to produce, elicit, or cause a greater physiological response (i.e., downstream effects) compared to the response caused by either vehicle or a control molecule/composition. A measurable physiological response may include an increase in T cell expansion, activation, persistence, and/or an increase in cancer cell death killing ability, among others apparent from the understanding in the art and the description herein. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. A “decrease” or “reduced” amount is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

By “maintain,” or “preserve,” or “maintenance,” or “no change,” or “no substantial change,” or “no substantial decrease” refers generally to the ability of a composition contemplated herein to produce, elicit, or cause a lesser physiological response (i.e., downstream effects) in a cell, as compared to the response caused by either vehicle, a control molecule/composition, or the response in a particular cell lineage. A comparable response is one that is not significantly different or measurable different from the reference response.

### Overview

In some aspects, the present disclosure is directed to compositions and methods for treating cancer using chimeric antigen receptor (CAR) cell therapy. More particularly, the present disclosure concerns CAR cell therapies in which the transformed cells, such as T cells, express CARs that target CLDN18.2. Still further, the CAR constructs, transformed cells expressing the constructs, and the therapies utilizing the transformed cells disclosed herein can provide robust cancer treatments against cancer expressing CLDN18.2. The present disclosure relates to culturing methods of T cells transduced with chimeric antigen receptors (CARs) that generate a persisting population of T cells that exhibit increased antigen-independent activation.

Without wishing to be bound by theory, CLDN18.2 is believed to be a viable cancer target across multiple modalities, including bispecific T cell engagers, CAR cells, as well as monoclonal antibodies and antibody-drug conjugates (ADCs). Further, it is believed that CLDN18.2 is a promising target for CAR cell therapy. Therefore, antibodies and CAR constructs derived from these antibodies have been developed as described herein.

### CAR Construct Design

CAR constructs of the present disclosure can have several components, many of which can be selected based upon a desired or refined function of the resultant CAR construct. In addition to an antigen binding domain, CAR constructs can have a spacer domain, a hinge domain, a signal peptide domain, a transmembrane domain, and one or more intracellular domains (for example, one or more costimulatory domains). In some embodiments, a CAR can optionally comprise an armoring domain comprising a nucleic acid sequence encoding an armoring molecule. Selection of one component over another (i.e., selection of a specific costimulatory domain from one receptor versus a costimulatory domain from a different receptor) can influence clinical efficacy and safety profiles.

Antigen Binding Domain

Antigen binding domains contemplated herein can include antibodies or one or more antigen-binding fragments thereof. One contemplated CAR construct targeting CLDN18.2 comprises a single chain variable fragment (scFv) containing light and heavy chain variable regions from one or more antibodies specific for CLDN18.2 that are either directly linked together or linked together via a flexible linker (e.g., a repeat of G45 having 1, 2, 3 or more repeats).

The antigen binding domain of a CAR targeting CLDN18.2 as disclosed herein can vary in its binding affinity for the CLDN18.2 protein. The relationship between binding affinity and efficacy can be more nuanced in the context of CARs as compared with antibodies, for which higher affinity is typically desirable. For example, preclinical studies on a receptor tyrosine kinase-like orphan receptor 1 (ROR1)-CAR derived from a high-affinity scFv (with a dissociation constant of 0.56 nM) resulted in an increased therapeutic index when compared with a lower-affinity variant. Conversely, other examples have been reported that engineering the scFv for lower affinity improves the discrimination among cells with varying antigen density. This could be useful for improving the therapeutic specificity for antigens differentially expressed on tumor versus normal tissues.

A variety of methods can be used to ascertain the binding affinity of the antigen binding domain. In some embodiments, methodologies that exclude avidity effects can be used. Avidity effects involve multiple antigen-binding sites simultaneously interacting with multiple target epitopes, often in multimerized structures. Thus, avidity functionally represents the accumulated strength of multiple interactions. An example of a methodology that excludes avidity effects is any approach in which one or both of the interacting proteins is monomeric/monovalent since multiple simultaneous interactions are not possible if one or both partners contain only a single interaction site.

Spacer Domain

A CAR construct of the present disclosure can have a spacer domain to provide conformational freedom to facilitate binding to the target antigen on the target cell. The optimal length of a spacer domain may depend on the proximity of the binding epitope to the target cell surface. For example, proximal epitopes can require longer spacers and distal epitopes can require shorter ones. Besides promoting binding of the CAR to the target antigen, achieving an optimal distance between a CAR cell and a cancer cell may also help to sterically occlude large inhibitory molecules from the immunological synapse formed between the CAR cell and the target cancer cell. A CAR targeting CLDN18.2 can have a long spacer, an intermediate spacer, or a short spacer. Long spacers can include a CH2CH3 domain (˜220 amino acids) of immunoglobulin G1 (IgG1) or IgG4 (either native or with modifications common in therapeutic antibodies, such as a S228P mutation), whereas the CH3 region can be used on its own to construct an intermediate spacer (˜120 amino acids). Shorter spacers can be derived from segments (<60 amino acids) of CD28, CD8α, CD3 or CD4. Short spacers can also be derived from the hinge regions of IgG molecules. These hinge regions may be derived from any IgG isotype and may or may not contain mutations common in therapeutic antibodies such as the S228P mutation mentioned above. For example, a hinge domain can comprise an IgG1 hinge domain or variants thereof, an IgG2 hinge domain or variants thereof, an IgG3 hinge domain or variants thereof, an IgG4 hinge domain or variants thereof, a CD8 hinge domain or variants thereof or a CD28 hinge domain or variants thereof.

Hinge Domain

A CAR targeting CLDN18.2 can also have a hinge domain. The flexible hinge domain is a short peptide fragment that provides conformational freedom to facilitate binding to the target antigen on the tumor cell. It may be used alone or in conjunction with a spacer sequence. The terms “hinge” and “spacer” are often used interchangeably—for example, IgG4 sequences can be considered both “hinge” and “spacer” sequences (i.e., hinge/spacer sequences). In some embodiments, a hinge domain can comprise an IgG1 hinge domain or variants thereof, an IgG2 hinge domain or variants thereof, an IgG3 hinge domain or variants thereof, an IgG4 hinge domain or variants thereof (in particular, an IgG4P hinge domain), a CD8 hinge domain or variants thereof or a CD28 hinge domain or variants thereof.

A CAR targeting CLDN18.2 can further include a sequence comprising a signal peptide. Signal peptides function to prompt a cell to translocate the CAR to the cellular membrane. Examples include an IgG1 heavy chain signal polypeptide, Ig kappa or lambda light chain signal peptides, granulocyte-macrophage colony stimulating factor receptor 2 (GM-CSFR2 or CSFR2) signal peptide, a CD8a signal polypeptide, or a CD33 signal peptide.

Transmembrane Domain

A CAR targeting CLDN18.2 can further include a sequence comprising a transmembrane domain. The transmembrane domain can include a hydrophobic α helix that spans the cell membrane. The properties of the transmembrane domain have not been as meticulously studied as other aspects of CAR constructs, but they can potentially affect CAR expression and association with endogenous membrane proteins. Transmembrane domains can be derived, for example, from CD4, CD8α, or CD28. Any transmembrane domain can be used in the compositions disclosed herein. In some embodiments, the transmembrane domain comprises a transmembrane domain selected from the transmembrane domain of CD3, CD4, CD8α, or CD28. In some embodiments, the transmembrane domain comprises a CD28 transmembrane domain.

Intracellular Domain/Costimulatory Domain

A CAR targeting CLDN18.2 can further include one or more sequences that form an intracellular domain and/or a costimulatory domain (also sometimes referred to as a signaling domain). A costimulatory domain is a domain capable of potentiating or modulating the response of immune effector cells (i.e., capable of initiating the response of immune effector cells). In some embodiments, the costimulatory domains and/or signaling domains can be derived from the intracellular T cell receptor (TCR) signalling domain (for example, the cytoplasmic domains of CD3ζ which contains sequence motifs called immunoreceptor tyrosine-based activation motifs (ITAMs)). Costimulatory domains can include sequences, for example, from one or more of CD3ζ (CD3z or CD3zeta), CD28, 4-1BB, OX-40, ICOS, CD27, GITR, CD2, IL-2Rβ and MyD88/CD40. In some embodiments, the costimulatory domain, can include variants of one or more of CD3ζ (CD3z or CD3zeta), CD28, 4-1BB, OX-40, ICOS, CD27, GITR, CD2, IL-2Rβ and MyD88/CD40. For example, in an embodiment, a CAR costimulatory domain can further include modifications to the CD3z domain. For instance, the CD3z signaling domain variants may contain 1 or 2 functional immunoreceptor tyrosine-based activation motifs (ITAMs) of the three ITAMs present in wild-type CD3z. The choice of costimulatory domain influences the phenotype and metabolic signature of CAR cells. For example, CD28 costimulation yields a potent, yet short-lived, effector-like phenotype, with high levels of cytolytic capacity, interleukin-2 (IL-2) secretion, and glycolysis. By contrast, T cells modified with CARs bearing 4-1BB costimulatory domains tend to expand and persist longer in vivo, have increased oxidative metabolism, are less prone to exhaustion, and have an increased capacity to generate central memory T cells. In some embodiments, the intracellular signaling domain comprises a costimulatory domain or a portion thereof.

In some embodiments, the intracellular domain comprises a costimulatory domain selected from the group consisting of the intracellular domain of a CD28 costimulatory domain, a CD27 costimulatory domain, a 4-1BB costimulatory domain, an ICOS costimulatory domain, an OX-40 costimulatory domain, a GITR costimulatory domain, a CD2 costimulatory domain, an IL-2Rβ costimulatory domain, a MyD88/CD40 costimulatory domain, and any combination thereof.

In certain embodiments, the intracellular domain comprises a costimulatory domain comprising a portion of the intracellular T cell receptor (TCR) signaling domain, CD3zeta (or CD3z; the CD3z signaling domain is also referred to herein as a “CD3z costimulatory domain”). In some embodiments, the CD3zeta comprises one or more modifications to the CD3z format. For example, CD3z signaling domain variants may contain 1 or 2 functional immunoreceptor tyrosine-based activation motifs (ITAMs) of the three ITAMs present in wild-type CD3z (e.g. 1XX, X1X, or X2X).

Exemplary CAR

According to all aspects of the invention, the CAR may comprise or consist of the amino acid sequence set forth as SEQ ID NO: 52. According to all aspects of the invention, the nucleic acid CAR construct may comprise or consist of the nucleic acid sequence set forth as SEQ ID NO: 51.

Armoring

In some embodiments, the CAR T cell (including TCR T cell) of the disclosure may be an “armored” CAR T cell, which is transformed with a CAR construct comprising one or more armoring domains encoding one or more armoring molecules and/or an independent construct comprising one or more armoring domains encoding one or more armoring molecules (e.g. such that the transformed cell expresses the CAR protein along with the one or more armoring molecules, such as cytokines to modulate the cytokine milieu of the tissue microenvironment). An “armoring molecule” refers to a protein that counters immunosuppression of a cell in a tumor microenvironment when expressed on a surface of the cell or when excreted in a tumor microenvironment and can provide many additional benefits not described herein that allow for T cell survival in the immunosuppressive tumor microenvironment (TME). In some embodiments, expression of the armoring molecule can be inducible or constitutive. In some embodiments, the armoring molecule is expressed on the surface of the cell. In some embodiments, the armoring molecule is secreted outside of the cell to armor CAR T cells. Expression of the armoring molecule on the cell surface and/or excretion to the TME can improve efficacy and persistence for the CAR T cells. In certain embodiments, certain genes encoding armoring molecules can be knocked out or their expression effectively eliminated (for example, using CRISPR) in order to improve efficacy and persistence for the CAR T cells in the TME. In this context, such CAR T cells are also referred to as “armored CAR T cells”. The armoring molecule may be selected based on the tumor microenvironment and other elements of the innate and adaptive immune systems. In certain embodiments, the armoring molecule is selected from a dominant negative TGFβ receptor type II, IL-7, IL-12, IL-15, IL-18, a hybrid IL-4/IL-7 receptor, hybrid IL-7/IL-2 receptor, and HIF1α dominant-negative. Furthermore, investigators reported modifying CAR-T cells to secrete PD-1-blocking single-chain variable fragments (scFv), which improved CAR-T cell anti-tumor activity in mouse models of PD-L1+ hematologic and solid tumors (Rafiq, S., Yeku, O., Jackson, H. et al. Targeted delivery of a PD-1-blocking scFv by CAR-T cells enhances anti-tumor efficacy in vivo. Nat Biotechnol 36, 847-856 (2018)). In some embodiments, the armoring molecule comprises a dominant-negative TGFβ receptor type 2 (dnTGFβRII). In certain embodiments, the armoring molecule comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 54. In some embodiments, the armoring molecule comprises the amino acid sequence set forth in SEQ ID NO: 54.

In some embodiments, a CAR nucleic acid construct can comprise an armoring domain comprising a nucleic acid sequence encoding an armoring molecule (e.g. SEQ ID NO: 53). In certain embodiments, the armoring domain is located at the 3′ end of the nucleic acid encoding the CAR or at the 5′ end of the nucleic acid encoding the CAR. In some embodiments, the CAR and the armoring domain are operably linked under the control of a single promoter. In some embodiments, the CAR and the armoring domain are operably linked by an internal ribosome entry site (IRES). In some embodiments, the CAR and the armoring domain are linked by a nucleotide sequence encoding a cleavable peptide linker (for example, a self-cleaving peptide linker). In certain embodiments, the cleavable peptide linker comprises a T2A peptide. As referred to herein, 2A self-cleaving peptides, or 2A peptides, are a class of 18-22 aa-long peptides, which can induce ribosomal skipping during translation of a protein in a cell. Examples can include, but are not limited to, P2A (ATNFSLLKQAGDVEENPGP; SEQ ID NO:69), E2A (QCTNYALLKLAGDVESNPGP; SEQ ID NO:70), F2A (VKQTLNFDLLKLAGDVESNPGP; SEQ ID NO:71), and T2A (EGRGSLLTCGDVEENPGP; SEQ ID NO:72). Thus, in such embodiments, whilst the CAR nucleic acid construct and armoring domain may be incorporated into the same nucleic acid vector and/or operably linked, upon transcription and translation, the CAR and armoring molecule (encoded by the armoring domain) may be expressed as independent proteins.

Exemplary Armored CAR

According to all armored aspects of the invention, the CAR may comprise or consist of the amino acid sequence set forth as SEQ ID NO: 52 and the armoring molecule may comprise or consist of the amino acid sequence set forth as SEQ ID NO: 54. According to all armored aspects of the invention, the nucleic acid CAR construct may encode an armored CAR sequence with the amino acid sequence set forth as SEQ ID NO: 56. In a particular embodiment, the nucleic acid CAR construct may comprise or consist of the nucleic acid sequence set forth as SEQ ID NO: 55.

### CAR Construct Assessment

Constructs of the present disclosure were compared and assessed based on safety as well as persistence and establishment of central memory. The lower affinity (high off-rate) scFv, 008LYG_D08, was assessed favorably on account of its improved safety. The CD3z signaling domain and CD28 costimulatory domains (both in the same construct) were assessed favorably based on their contribution to improved persistence and favorable in vivo phenotype (more central memory). The CLDN18.2 CARs of the present disclosure compared favorably to constructs based on published CLDN18.2-targeting CARs. Details of the assessment can be found in the Examples.

### CAR Cell Production

CAR constructs of the present disclosure can include some combination of the modular components described herein. For example, in some embodiments of the present disclosure, a CAR construct comprises a CLDN18.2 scFv antigen binding domain. In some embodiments of the present disclosure, a CAR construct comprises a CSFR2 signal peptide. In some embodiments, a CAR construct comprises an IgG4 hinge/spacer domain carrying an S241P mutation (IgG4P). In some embodiments, a CAR construct comprises a CD28 transmembrane domain.

Different costimulatory domains can be utilized in the CAR constructs of the present disclosure. In some embodiments, a CAR construct comprises a costimulatory domain comprising a signaling domain from the intracellular domain of CD3z (for example, a portion of the intracellular T cell receptor (TCR) signaling domain, CD3zeta (or CD3z) or a variant thereof). In some embodiments, a CAR construct comprises a CD28 costimulatory domain. In some embodiments, a CAR construct comprises a 4-1BB costimulatory domain. In some embodiments, a CAR construct comprises costimulatory domains from CD3z and CD28, as described herein. In some embodiments, a CAR construct comprises costimulatory domains from CD3z and 4-1BB, as described herein. In some embodiments, a CAR construct comprises costimulatory domains from all of CD3z, CD28, and 4-1BB, as described herein. In some embodiments, a CAR construct comprises costimulatory domains from ICOS, OX-40, and/or GITR.

### Cells

CAR-based cell therapies can be used with a variety of cell types, such as lymphocytes. Particular types of cells that can be used include T cells, Natural Killer (NK) cells, Natural Killer T (NKT) cells, Invariant Natural Killer T (iNKT) cells, alpha beta T cells, gamma delta T cells, viral-specific T (VST) cells, cytotoxic T lymphocytes (CTLs), and regulatory T cells (Tregs). In one embodiment, CAR cells for treating a subject are autologous. In other embodiments, the CAR cells may be from a genetically similar, but non-identical donor (allogeneic).

### SMART

The present disclosure also relates to culturing methods of T cells transduced with chimeric antigen receptors (CARs) that generate a persisting population of T cells that exhibit increased antigen-independent activation. The acronym “SMART” (Shorty-Manipulated Auto-Replicating T-Cells) refers to a shortened T cell manufacture and expansion process wherein the cells are cultured in the presence of IL-21 (and optionally IL-2).

Some aspects of the present disclosure are directed to cells comprising a polynucleotide or a polypeptide disclosed herein. Some aspects of the present disclosure are directed to a cell comprising (i) a polynucleotide encoding a chimeric antigen receptor (CAR) that binds human CLDN18.2. In some embodiments, the cell further comprises (ii) a polynucleotide encoding an armoring molecule. In some embodiments, the cell is an immune cell. In some embodiments, the cell is an autologous cell to the recipient. In some embodiments, the cell is selected from the group consisting of a T cell, a Natural Killer (NK) cell, a cytotoxic T lymphocyte (CTL), a regulatory T cell, a γδ T cell, a TSCM cell, a CMV+ T cell, a tumor infiltrating lymphocyte, and any combination thereof. In some embodiments, the cell is a mammalian cell. In some embodiments, the cell is a human cell.

Prior to expansion and genetic modification of the T cells of the disclosure, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell lines available in the art, may be used. In certain embodiments of the present disclosure, T cells can be obtained from a unit of blood collected from a subject using any number of techniques known to the skilled artisan, such as Ficoll™ separation. In one embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In some embodiments, the cells are washed with phosphate buffered saline (PBS). In an alternative embodiment, the wash solution lacks calcium and may lack magnesium or may lack many if not all divalent cations. Again, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca2+-free, Mg2+-free PBS, PlasmaLyte A, or other saline solution with or without buffer. Alternatively, the undesirable components of the apheresis sample may be removed and the cells directly resuspended in culture media.

In other embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. In some embodiments, T cells are isolated by positive selection for CD4 and CD8 expression. For example, in one embodiment, T cells are isolated by incubation with anti-CD4/anti-CD8-conjugated beads for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, by simply shortening or lengthening the time T cells are allowed to bind to the CD4/CD8 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD4 and/or anti-CD8 antibodies on the beads or other surface, subpopulations of T cells can be preferentially selected for or against at culture initiation or at other desired time points. The skilled artisan would recognize that multiple rounds of selection can also be used in the context of this disclosure. In certain embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, and HLA-DR. In certain embodiments, it may be desirable to enrich for or positively select for regulatory T cells which typically express CD4+, CD25+, CD62Lhi, GITR+, and FoxP3+. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature.

T cells for stimulation can also be frozen after a washing step. In some embodiments, the freeze and subsequent thaw step can provide a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed and allowed to rest for one hour at room temperature prior to activation using the methods of the present disclosure.

Also contemplated in the context of this disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In a further embodiment of the present disclosure, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in certain embodiments, mobilization (for example, mobilization with GM-CSF or G-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

Activation and Expansion of T Cells

Whether prior to or after genetic modification of the T cells to express a desirable CAR, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the disclosure are expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For costimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD28 antibody include 9.3, B-T3, XR-CD28 (Diaclone, Besangon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain embodiments, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the costimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present disclosure.

In one embodiment, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof, and both agents are co-immobilized to the same bead in equivalent molecular amounts. In one embodiment, a 1:1 ratio of each antibody bound to the beads for CD4+ T cell expansion and T cell growth is used. In certain embodiments of the present disclosure, a ratio of anti CD3:CD28 antibodies bound to the beads is used such that an increase in T cell expansion is observed as compared to the expansion observed using a ratio of 1:1.

In further embodiments of the present disclosure, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), IL-21, insulin, IFN-7, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2). In one embodiment, the media is X-VIVO 15 serum-free media containing 1% (v/v) recombinant serum replacement (ITSE-A).

In one embodiment, the T cells are cultured in media containing between 10 and 300 IU/mL of recombinant human IL-2. In one embodiment, the T cells are cultured in media containing 10, 15, 20, 25, 30, 35, 40, 45, 50, 55, 60, 65, 70, 75, 80, 85, 90, 95, 100, 200, or 300 IU/mL of recombinant human IL-2. In another embodiment, the T cells are cultured in media also containing between 0.1 and 0.3 U/mL of recombinant IL-21. In another embodiment, the T cells are cultured in media containing IL-2 and 0.1, 0.2, 0.5, 1, 2, 5, 10, 15, 20, 25, 30, 40, 50, 75, or 100 U/mL of recombinant human IL-21. In another embodiment, the T cells are culture in media containing IL-2 and 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, or 0.30 U/mL of recombinant human IL-21. In one embodiment, the T cells are cultured in a media containing 40 IU/mL of recombinant human IL-2 and 0.24 U/mL of recombinant human IL-21.

In one embodiment of the present disclosure, the cells cultured for up to 14 days. In another embodiment, the mixture may be cultured for 4 days. The T cells can be agitated during any stage of culture. In one embodiment, the cells are agitated during cell culture in media containing IL-2 and IL-21. In certain embodiments, the T cells harvested on day 4 exhibit higher target independent killing activity compared to CAR-T cells harvested on day 6.

### Anti-CLDN18.2 Antibodies of the Present Disclosure

Some aspects of the present disclosure are directed to antibodies or antigen-binding portions thereof that specifically binds human CLDN18.2. In some embodiments, the antibody or antigen-binding portion thereof comprises a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3. In some embodiments, the antibody or an antigen-binding portion thereof that specifically binds CLDN18.2 comprises a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3, wherein
(a) the VH-CDR1 comprises an amino acid sequence selected from SEQ ID NOs: 1, 11, 21, 31, and 41;
(b) the VH-CDR2 comprises an amino acid sequence selected from SEQ ID NOs: 2, 12, 22, 32, and 42;
(c) the VH-CDR3 comprises an amino acid sequence selected from SEQ ID NOs: 3, 13, 23, 33, and 43;
(d) the VL-CDR1 comprises an amino acid sequence selected from SEQ ID NOs: 4, 14, 24, 34, and 44;
(e) the VL-CDR2 comprises an amino acid sequence selected from SEQ ID NOs: 5, 15, 25, 35, and 45; and
(f) the VL-CDR3 comprises an amino acid sequence selected from SEQ ID NOs: 6, 16, 26, 36, and 46.

The VH may comprise an amino acid sequence selected from SEQ ID NOs: 7, 17, 27, 37 and 47. The VL may comprise an amino acid sequence selected from SEQ ID NOs: 8, 18, 28, 38 and 48.

In some embodiments, the antibody or an antigen-binding portion comprises a VH-CDR1, a VH-CDR2, a VH-CDR3; and a VL-CDR1, a VL-CDR2, and VL-CDR3, wherein:
(a) VH-CDR1 comprises the amino acid sequence set forth in SEQ ID NO: 1, VH-CDR2 comprises the amino acid sequence set forth in SEQ ID NO: 2, VH-CDR3 comprises the amino acid sequence set forth in SEQ ID NO: 3, VL-CDR1 comprises the amino acid sequence set forth in SEQ ID NO: 4, VL-CDR2 comprises the amino acid sequence set forth in SEQ ID NO: 5, and VL-CDR3 comprises the amino acid sequence set forth in SEQ ID NO: 6;
(b) VH-CDR1 comprises the amino acid sequence set forth in SEQ ID NO: 11, VH-CDR2 comprises the amino acid sequence set forth in SEQ ID NO: 12, VH-CDR3 comprises the amino acid sequence set forth in SEQ ID NO: 13, VL-CDR1 comprises the amino acid sequence set forth in SEQ ID NO: 14, VL-CDR2 comprises the amino acid sequence set forth in SEQ ID NO: 15, and VL-CDR3 comprises the amino acid sequence set forth in SEQ ID NO: 16;
(c) VH-CDR1 comprises the amino acid sequence set forth in SEQ ID NO: 21, VH-CDR2 comprises the amino acid sequence set forth in SEQ ID NO: 22, VH-CDR3 comprises the amino acid sequence set forth in SEQ ID NO: 23, VL-CDR1 comprises the amino acid sequence set forth in SEQ ID NO: 24, VL-CDR2 comprises the amino acid sequence set forth in SEQ ID NO: 25, and VL-CDR3 comprises the amino acid sequence set forth in SEQ ID NO: 26;
(d) VH-CDR1 comprises the amino acid sequence set forth in SEQ ID NO: 31, VH-CDR2 comprises the amino acid sequence set forth in SEQ ID NO: 32, VH-CDR3 comprises the amino acid sequence set forth in SEQ ID NO: 33, VL-CDR1 comprises the amino acid sequence set forth in SEQ ID NO: 34, VL-CDR2 comprises the amino acid sequence set forth in SEQ ID NO: 35, and VL-CDR3 comprises the amino acid sequence set forth in SEQ ID NO: 36; or
(e) VH-CDR1 comprises the amino acid sequence set forth in SEQ ID NO: 41, VH-CDR2 comprises the amino acid sequence set forth in SEQ ID NO: 42, VH-CDR3 comprises the amino acid sequence set forth in SEQ ID NO: 43, VL-CDR1 comprises the amino acid sequence set forth in SEQ ID NO: 44, VL-CDR2 comprises the amino acid sequence set forth in SEQ ID NO: 45, and VL-CDR3 comprises the amino acid sequence set forth in SEQ ID NO: 46.

In some embodiments, the antibody or an antigen-binding portion comprises a VH and a VL, wherein:
(a) the VH comprises the amino acid sequence set forth in SEQ ID NO: 7 and the VL comprises the amino acid sequence set forth in SEQ ID NO: 8, optionally wherein the antibody or an antigen-binding portion comprises an scFv comprising the amino acid sequence set forth in SEQ ID NO: 9;
(b) the VH comprises the amino acid sequence set forth in SEQ ID NO: 17 and the VL comprises the amino acid sequence set forth in SEQ ID NO: 18, optionally wherein the antibody or an antigen-binding portion comprises an scFv comprising the amino acid sequence set forth in SEQ ID NO: 19;
(c) the VH comprises the amino acid sequence set forth in SEQ ID NO: 27 and the VL comprises the amino acid sequence set forth in SEQ ID NO: 28, optionally wherein the antibody or an antigen-binding portion comprises an scFv comprising the amino acid sequence set forth in SEQ ID NO: 29;
(d) the VH comprises the amino acid sequence set forth in SEQ ID NO: 37 and the VL comprises the amino acid sequence set forth in SEQ ID NO: 38, optionally wherein the antibody or an antigen-binding portion comprises an scFv comprising the amino acid sequence set forth in SEQ ID NO: 39; or
(e) the VH comprises the amino acid sequence set forth in SEQ ID NO: 47 and the VL comprises the amino acid sequence set forth in SEQ ID NO: 48, optionally wherein the antibody or an antigen-binding portion comprises an scFv comprising the amino acid sequence set forth in SEQ ID NO: 49.

### Vectors, Host Cells, and Pharmaceutical Compositions of the Disclosure

In some embodiments, the polynucleotides of the present disclosure are present in a vector. As such, provided herein are vectors comprising the polynucleotides of the present disclosure. In some embodiments, the present disclosure is directed to a vector or a set of vectors comprising a polynucleotide encoding a CAR, as described herein. In other embodiments, the present disclosure is directed to a vector or a set of vectors comprising a polynucleotide encoding an armoring molecule, as disclosed herein. In other embodiments, the present disclosure is directed to a vector or a set of vectors comprising a polynucleotide encoding an antibody or an antigen binding molecule thereof that specifically binds to CLDN18.2, as disclosed herein.

In some embodiments, the set of vectors comprises a first vector and a second vector, wherein the first vector comprises a nucleic acid sequence encoding a CAR disclosed herein, and the second vector comprises a nucleic acid sequence encoding an armoring molecule disclosed herein. In other embodiments, the vector comprises both a nucleic acid sequence encoding a CAR disclosed herein and an armoring domain encoding an armoring molecule as defined herein.

Any vector known in the art can be suitable for the present disclosure. In some embodiments, the vector is a viral vector. In some embodiments, the vector is a retroviral vector, a DNA vector, a murine leukemia virus vector, an SFG vector, a plasmid, a RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector, transposon, or any combination thereof. In certain embodiments, the CARs and/or antibodies or antigen binding fragments thereof are encompassed by and/or delivered to a cell and or patient using a virus, a lentivirus, an adenovirus, a retrovirus, an adeno-associated virus (AAV), a transposon, a DNA vector, a mRNA, a lipid nanoparticle (LNP), or a CRISPR-Cas System.

In other embodiments, provided herein are host cells comprising a polynucleotide or a vector of the present disclosure. In some embodiments, the present disclosure is directed to host cells, e.g., in vitro cells, comprising a polynucleotide encoding a CAR or a TCR, as described herein. In some embodiments, the present disclosure is directed to host cells, e.g., in vitro cells, comprising a polynucleotide encoding an antibody or an antigen binding molecule thereof that specifically binds to CLDN18.2, as disclosed herein. In other embodiments, the present disclosure is directed to in vitro cells comprising a polypeptide encoded by a polynucleotide encoding a CAR that specifically binds to CLDN18.2. In other embodiments, the present disclosure is directed to cells, in vitro cells, comprising a polypeptide encoded by a polynucleotide encoding an antibody or an antigen binding molecule thereof that specifically binds to CLDN18.2, as disclosed herein.

Any cell can be used as a host cell for the polynucleotides, the vectors, or the polypeptides of the present disclosure. In some embodiments, the cell can be a prokaryotic cell, fungal cell, yeast cell, or higher eukaryotic cells such as a mammalian cell. Suitable prokaryotic cells include, without limitation, eubacteria, such as Gram-negative or Gram-positive organisms, for example, Enterobacteriaceae such as Escherichia, e.g., E. coli; Enterobacter; Erwinia; Klebsiella; Proteus; Salmonella, e.g., Salmonella typhimurium; Serratia, e.g., Serratia Marcescens, and Shigella; Bacilli such as B. subtilis and B. licheniformis; Pseudomonas such as P. aeruginosa; and Streptomyces. In some embodiments, the cell is a human cell.

Other embodiments of the present disclosure are directed to compositions comprising a polynucleotide described herein, a vector described herein, a polypeptide described herein, or cell described herein. In some embodiments, the composition comprises a pharmaceutically acceptable carrier, diluent, solubilizer, emulsifier, preservative and/or adjuvant. In some embodiments, the composition comprises an excipient. In one embodiment, the composition comprises a polynucleotide encoding a CAR, wherein the CAR comprises an antigen binding molecule that specifically binds to CLDN18.2. In another embodiment, the composition comprises a CAR encoded by a polynucleotide of the present disclosure, wherein the CAR comprises an antigen binding molecule that specifically binds to CLDN18.2. In another embodiment, the composition comprises a T cell comprising a polynucleotide encoding a CAR, wherein the CAR comprises an antigen binding molecule that specifically binds to CLDN18.2. In another embodiment, the composition comprises an antibody or an antigen binding molecule thereof that specifically binds CLDN18.2, as described herein. In another embodiment, the composition comprises a cell (e.g., a T cell, e.g., a CAR-T cell) comprising a polynucleotide encoding CAR comprising an antigen binding domain that specifically binds CLDN18.2, as disclosed herein.

In other embodiments, the composition is formulated for parenteral delivery, for inhalation, or for delivery through the digestive tract, such as orally. The preparation of such pharmaceutically acceptable compositions is within the ability of one skilled in the art. In certain embodiments, buffers are used to maintain the composition at physiological pH or at a slightly lower pH, typically within a pH range of from about 5 to about 8. In certain embodiments, when parenteral administration is contemplated, the composition is in the form of a pyrogen-free, parenterally acceptable aqueous solution with or without additional therapeutic agents, in a pharmaceutically acceptable vehicle. In certain embodiments, the vehicle for parenteral injection is sterile distilled water with or without at least one additional therapeutic agent, is formulated as a sterile, isotonic solution, properly preserved. In certain embodiments, the preparation involves the formulation of the desired molecule with polymeric compounds (such as polylactic acid or polyglycolic acid), beads or liposomes, that provide for the controlled or sustained release of the product, which are then be delivered via a depot injection. In certain embodiments, implantable drug delivery devices are used to introduce the desired molecule.

Treatment of Cancers with CARs

In some embodiments, the present disclosure provides CAR cells for treatment of cancer. The compositions (e.g., antibodies, CAR constructs, and CAR cells) and methods of their use described herein are especially useful for inhibiting neoplastic cell growth or spread; particularly neoplastic cell growth in which CLDN18.2 plays a role.

Neoplasms treatable by the compositions of the disclosure include solid tumors, for example, those of the liver, lung, or pancreas. However, the cancers listed herein are not intended to be limiting. For example, types of cancer that are contemplated for treatment herein include, for example, gastric cancer, gastroesophageal junction cancer (GEJ; e.g., distal oesophageal cancers, proximal gastric cancers and cancers of cardia), pancreatic cancer, breast cancer, colon cancer, liver cancer, head and neck cancer, bronchial cancer, or non-small-cell lung cancer.

In one embodiment, cancers contemplated for treatment here include any that express CLDN18.2 on the cell surfaces of the cancer cells. Cancers contemplated for treatment herein can include, but is not limited to, gastric cancer, gastroesophageal junction cancer (GEJ; e.g., distal oesophageal cancers, proximal gastric cancers and cancers of cardia), pancreatic cancer, breast cancer, colon cancer, liver cancer, head and neck cancer, bronchial cancer, biliary adenocarcinoma, ovarian cancer, hepatocellular carcinoma, and non-small-cell lung cancer.

### Methods of Treatment

CAR− modified cells of the present disclosure, such as CART cells, may be administered alone or as a pharmaceutical composition with a diluent and/or other components associated with cytokines or cell populations. Briefly, pharmaceutical compositions of the disclosure can include, for example CAR T cells as described herein, with one or more pharmaceutically or physiologically acceptable carrier, diluent, or excipient. Such compositions can comprise buffers such as neutral buffered saline, buffered saline, and the like; sulfates; carbohydrates such as glucose, mannose, sucrose, or dextrans, mannitol; proteins, polypeptides, or amino acids such as glycine; antioxidants; chelating agents such as EDTA or glutathione; adjuvants (e.g., aluminum hydroxide); and preservatives. The pharmaceutical compositions of the disclosure may be adapted to the treatment (or prophylaxis).

In some embodiments, the present disclosure provides a method of treating cancer including administering to a subject in need thereof an effective amount of a cell comprising an anti-CLDN18.2 chimeric antigen receptor (CAR) comprising an antigen binding domain. The antigen binding domain can be an antibody, Fab, or an scFv comprising a heavy chain variable region (VH) and a light chain variable region (VL). In certain embodiments, the VH comprises a CDR1 comprising an amino acid sequence selected from SEQ ID NO: 1, 11, 21, 31, and 41; a CDR2 comprising an amino acid sequence selected from SEQ ID NO: 2, 12, 22, 32, and 42; and a CDR3 comprising an amino acid sequence selected from SEQ ID NO: 3 13, 23, 33, and 43; and the VL comprises a CDR1 comprising an amino acid sequence selected from SEQ ID NO: 4, 14, 24, 34, and 44; a CDR2 comprising an amino acid sequence selected from SEQ ID NO: 5, 15, 25, 35, and 45; and a CDR3 comprising an amino acid sequence selected from SEQ ID NO: 6, 16, 26, 36, and 46. In certain embodiments, the VH comprises an amino acid sequence selected from SEQ ID NO: 7, 17, 27, 37 and 47. In some embodiments, the VL comprises an amino acid sequence selected from SEQ ID NO: 8, 18, 28, 38, and 48. In some embodiments, the method further inhibits tumor growth, induces tumor regression, and/or prolongs survival of the subject.

In some embodiments, the present disclosure provides a method of treatment that include administering to a subject in need thereof an effective amount of an anti-CLDN18.2 antibody or antigen binding fragment thereof. As used herein, an “effective amount” of the anti-CLDN 18.2 antibodies or antigen binding fragments thereof as disclosed herein (or a pharmaceutical formulation), refers to an amount effective, at dosages and for periods of time necessary, to achieve the desired therapeutic or prophylactic result.

In some embodiments, the cell is an autologous cell. For example, the autologous cell can be selected from the group consisting of a T cell, a Natural Killer (NK) cell, a cytotoxic T lymphocyte (CTL), and a regulatory T cell.

In some embodiments, the cancer treated by the method is a solid tumor. For example, the cancer can be gastric cancer, gastroesophageal junction cancer (GEJ; e.g., distal oesophageal cancers, proximal gastric cancers and cancers of cardia), pancreatic cancer, breast cancer, colon cancer, liver cancer, head and neck cancer, bronchial cancer, biliary adenocarcinoma, ovarian cancer, hepatocellular carcinoma, and non-small-cell lung cancer.

### Embodiments

In some embodiments, the present disclosure provides:

Embodiment 1. An isolated nucleic acid sequence encoding a chimeric antigen receptor (CAR), wherein the CAR comprises:
(a) an antigen binding domain specific for claudin 18.2 (CLDN18.2);
(b) a transmembrane domain; and
(c) one or more intracellular domains.

Embodiment 2. The isolated nucleic acid sequence of embodiment 1, wherein the antigen binding domain comprises an antibody or antigen-binding fragment thereof, Fab, Fab′, F(ab′)2, Fd, Fv, single-chain fragment variable (scFv), single chain antibody, VHH, vNAR, nanobody (single-domain antibody), or any combination thereof.

Embodiment 3. The isolated nucleic acid sequence of embodiment 2, wherein the antigen binding domain is a single chain variable fragment (scFv).

Embodiment 4. The isolated nucleic acid sequence of embodiment 3, wherein the antigen binding domain is a scFv comprising an amino acid sequence selected from SEQ ID NO: 9, 19, 29, 39, and 49.

Embodiment 5. The isolated nucleic acid sequence of any one of embodiments 1 to 4, wherein the transmembrane domain comprises a transmembrane domain selected from the transmembrane domain of CD4, CD8α, or CD28.

Embodiment 6. The isolated nucleic acid sequence of embodiment 5, wherein the transmembrane domain comprises a CD28 transmembrane domain.

Embodiment 7. The isolated nucleic acid sequence of any one of embodiments 1 to 6, wherein the one or more intracellular domains comprises a costimulatory domain or a portion thereof.

Embodiment 8. The isolated nucleic acid sequence of embodiment 7, wherein the costimulatory domain comprises one or more of CD3z, CD2, CD27, CD28, 4-1BB, OX-40, ICOS, IL-2Rβ, GITR, MyD88/CD40a costimulatory domains, and/or variants thereof.

Embodiment 9. The isolated nucleic acid sequence of any one of embodiments 1 to 8, wherein the intracellular domain comprises a CD3z costimulatory domain and a CD28 costimulatory domain.

Embodiment 10. The isolated nucleic acid sequence of any one of embodiments 1 to 8, wherein the intracellular domain comprises a CD3z costimulatory domain and a 4-1BB costimulatory domain.

Embodiment 11. The isolated nucleic acid sequence of any one of embodiments 1 to 8, wherein the intracellular domain comprises a CD3z costimulatory domain, a CD28 costimulatory domain, and a 4-1BB costimulatory domain.

Embodiment 12. The isolated nucleic acid sequence of any one of embodiments 1 to 11, wherein the CAR further comprises a hinge/spacer domain, optionally, wherein the hinge/spacer domain is located between the antigen binding domain and the transmembrane domain.

Embodiment 13. The isolated nucleic acid sequence of embodiment 12, wherein the hinge/spacer domain comprises an IgG1 hinge domain or variants thereof, an IgG2 hinge domain or variants thereof, an IgG3 hinge domain or variants thereof, an IgG4 hinge domain or variants thereof, an IgG4P domain, a CD8 hinge domain or variants thereof or a CD28 hinge domain or variants thereof.

Embodiment 14. The isolated nucleic acid sequence of embodiment 13, wherein the hinge/spacer domain is an IgG4 hinge/spacer, or variants thereof, optionally an IgG4P hinge/spacer comprising an S241P mutation.

Embodiment 15. The isolated nucleic acid sequence of any one of embodiments 1 to 14, wherein the nucleic acid sequence encodes a CAR with an amino acid sequence as set forth in SEQ ID NO: 52, optionally wherein the nucleic acid sequence is as set forth in SEQ ID NO: 51.

Embodiment 16. The isolated nucleic acid sequence of any one of embodiments 1 to 15, which further comprises an armoring domain comprising a nucleic acid sequence encoding an armoring molecule, optionally wherein the armoring domain is located at the 3′ end of the nucleic acid encoding the CAR or at the 5′ end of the nucleic acid encoding the CAR.

Embodiment 17. The isolated nucleic acid sequence of embodiment 16, wherein the armoring molecule is selected from a dominant-negative TGFβ receptor type II, IL-7, IL-12, IL-15, IL-18, a hybrid IL-4/IL-7 receptor, a hybrid IL-7/IL-2 receptor, and HIF1α dominant-negative.

Embodiment 18. The isolated nucleic acid sequence of embodiment 17, wherein the armoring molecule comprises a dominant-negative TGFβ receptor type II (dnTGFβRII).

Embodiment 19. The isolated nucleic acid sequence of either embodiment 17 or 18, wherein the armoring molecule comprises an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO: 54.

Embodiment 20. The isolated nucleic acid sequence of any one of embodiments 17 to 19, wherein the dominant negative TGFβ receptor type II comprises the sequence of SEQ ID NO:54, optionally wherein the armoring domain encoding the dnTGFβRII has the sequence as set forth in SEQ ID NO: 53.

Embodiment 21. The isolated nucleic acid sequence of any one of embodiments 1 to 20, wherein the CAR and the armoring domain are operably linked under the control of a single promoter.

Embodiment 22. The isolated nucleic acid sequence of any one of embodiments 1 to 20, wherein the CAR and the armoring domain are operably linked by an internal ribosome entry site (IRES).

Embodiment 23. The isolated nucleic acid sequence of any one of embodiments 1 to 22, wherein the CAR and the armoring domain are linked by a nucleotide sequence encoding a cleavable peptide linker.

Embodiment 24. The isolated nucleic acid sequence of embodiment 23, wherein the cleavable peptide linker is a self-cleaving peptide linker.

Embodiment 25. The isolated nucleic acid sequence of either embodiment claim 23 or 24, wherein the cleavable peptide linker comprises a T2A peptide.

Embodiment 26. The isolated nucleic acid sequence of any one of embodiments 1 to 25, wherein the nucleic acid sequence encodes a sequence selected from SEQ ID NO: 55, 10, 20, 30, 40, and 50.

Embodiment 27. An anti-CLDN18.2 chimeric antigen receptor (CAR) comprising an antigen binding domain, wherein the antigen binding domain comprises an antibody, Fab, or an scFv comprising a heavy chain variable region (VH) and a light chain variable region (VL);
wherein the VH comprises a CDR1 comprising an amino acid sequence selected from SEQ ID NO: 1, 11, 21, 31, and 41; a CDR2 comprising an amino acid sequence selected from SEQ ID NO: 2, 12, 22, 32, and 42; and a CDR3 comprising an amino acid sequence selected from SEQ ID NO: 3 13, 23, 33, and 43; and
wherein the VL comprises a CDR1 comprising an amino acid sequence selected from SEQ ID NO: 4, 14, 24, 34, and 44; a CDR2 comprising an amino acid sequence selected from SEQ ID NO: 5, 15, 25, 35, and 45; and a CDR3 comprising an amino acid sequence selected from SEQ ID NO: 6, 16, 26, 36, and 46.

Embodiment 28. The anti-CLDN18.2 CAR of embodiment 27, wherein the VH comprises an amino acid sequence selected from SEQ ID NO: 7, 17, 27, 37 and 47.

Embodiment 29. The anti-CLDN18.2 CAR of either embodiment 27 or 28, wherein the VL comprises an amino acid sequence selected from SEQ ID NO: 8, 18, 28, 38, and 48.

Embodiment 30. The anti-CLDN18.2 CAR of embodiment 27 to 29, wherein the CAR comprises a transmembrane domain, and one or more intracellular domains.

Embodiment 31. The anti-CLDN18.2 CAR of any one of embodiments 27 to 30, wherein the transmembrane domain comprises a transmembrane domain selected from the transmembrane domain of CD4, CD8α, or CD28.

Embodiment 32. The anti-CLDN18.2 CAR of embodiment 31, wherein the transmembrane domain comprises a CD28 transmembrane domain.

Embodiment 33. The anti-CLDN18.2 CAR of any one of embodiments 27 to 32, wherein the one or more intracellular domains comprises a costimulatory domain or a portion thereof.

Embodiment 34. The anti-CLDN18.2 CAR of embodiment 33, wherein the costimulatory domain comprises one or more of CD3z, CD2, CD27, CD28, 4-1BB, OX-40, ICOS, IL-2Rβ, GITR, MyD88/CD40a costimulatory domains, and/or variants thereof.

Embodiment 35. The anti-CLDN18.2 CAR of any one of embodiments 30 to 34, wherein the intracellular domain comprises a CD3z costimulatory domain and a CD28 costimulatory domain.

Embodiment 36. The anti-CLDN18.2 CAR of any one of embodiments 30 to 34, wherein the intracellular domain comprises a CD3z costimulatory domain and a 4-1BB costimulatory domain.

Embodiment 37. The anti-CLDN18.2 CAR of any one of embodiments 30 to 34, wherein the intracellular domain comprises a CD3z costimulatory domain, a CD28 costimulatory domain, and a 4-1BB costimulatory domain.

Embodiment 38. The anti-CLDN18.2 CAR of any one of embodiments 27 to 37, wherein the CAR further comprises a hinge/spacer domain, optionally, wherein the hinge/spacer domain is located between the antigen binding domain and the transmembrane domain.

Embodiment 39. The anti-CLDN18.2 CAR of embodiment 38, wherein the hinge/spacer domain comprises an IgG1 hinge domain or variants thereof, an IgG2 hinge domain or variants thereof, an IgG3 hinge domain or variants thereof, an IgG4 hinge domain or variants thereof, an IgG4P domain, a CD8a hinge domain or variants thereof, or a CD28 hinge domain or variants thereof.

Embodiment 40. The anti-CLDN18.2 CAR of embodiment 39, wherein the hinge/spacer domain is an IgG4 hinge/spacer, or variants thereof, optionally an IgG4P hinge/spacer comprising an S241P mutation.

Embodiment 41. The anti-CLDN18.2 CAR of any one of embodiments 27 to 40, wherein the CAR has an amino acid sequence as set forth in SEQ ID NO: 52.

Embodiment 42. The anti-CLDN18.2 CAR of any one of embodiments 27 to 40, wherein the CAR further comprises an armoring molecule.

Embodiment 43. The anti-CLDN18.2 CAR of embodiment 42, wherein the armoring molecule is selected from a dominant-negative TGFβ receptor type II, IL-7, IL-12, IL-15, IL-18, a hybrid IL-4/IL-7 receptor, a hybrid IL-7/IL-2 receptor, and HIF1α dominant-negative.

Embodiment 44. The anti-CLDN18.2 CAR of embodiment 43, wherein the armoring molecule comprises a dominant-negative TGFβ receptor type II (dnTGFβRII).

Embodiment 45. The anti-CLDN18.2 CAR of either embodiment 43 or 44, wherein the armoring molecule comprises an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO:54.

Embodiment 46. The anti-CLDN18.2 CAR of any one of embodiments 43 to 45, wherein the dominant negative TGFβ receptor type II comprises the sequence of SEQ ID NO:54.

Embodiment 47. The anti-CLDN18.2 CAR of any one of embodiments 27 to 46, wherein the CAR and the armoring molecule are linked by a nucleotide sequence encoding a cleavable peptide linker.

Embodiment 48. The anti-CLDN18.2 CAR of embodiment 47, wherein the cleavable peptide linker is a self-cleaving peptide linker.

Embodiment 49. The anti-CLDN18.2 CAR of either embodiment 47 or 48, wherein the cleavable peptide linker comprises a T2A peptide.

Embodiment 50. The anti-CLDN18.2 CAR of any one of embodiments 27-49, wherein the CAR comprises an amino acid sequence selected from SEQ ID NO: 56, 10, 20, 30, 40, and 50.

Embodiment 51. A vector comprising the isolated nucleic acid sequence of any one of embodiments 1-26 or encoding the chimeric antigen receptor of any one of embodiments 27-50, optionally wherein the vector is a virus, a lentivirus, an adenovirus, a retrovirus, an adeno-associated virus (AAV), a transposon, a DNA vector, a mRNA, a lipid nanoparticle (LNP), or a CRISPR-Cas System, optionally wherein the vector is a lentivirus.

Embodiment 52. A cell comprising the vector of embodiment 51.

Embodiment 53. A cell comprising a nucleic acid sequence encoding the chimeric antigen receptor (CAR) of any one of embodiments 27-50, preferably wherein the cell comprises a nucleic acid sequence encoding a CAR with an amino acid sequence as set forth in SEQ ID NO: 52 and a nucleic acid encoding a dominant negative TGFβ receptor type II with a sequence as set forth in SEQ ID NO:54, optionally wherein the nucleic acid sequence encoding the CAR is as set forth in SEQ ID NO: 51 and the sequence encoding the dominant negative TGFβ receptor type II is as set forth in SEQ ID NO: 53.

Embodiment 54. A cell comprising a CLDN18.2 specific antigen binding domain, wherein the antigen binding domain comprises an antibody, Fab, or an scFv comprising a heavy chain variable region (VH) and a light chain variable region (VL);
wherein the VH comprises a CDR1 comprising an amino acid sequence selected from SEQ ID NO: 1, 11, 21, 31, and 41; a CDR2 comprising an amino acid sequence selected from SEQ ID NO: 2, 12, 22, 32, and 42; and a CDR3 comprising an amino acid sequence selected from SEQ ID NO: 3 13, 23, 33, and 43; and
wherein the VL comprises a CDR1 comprising an amino acid sequence selected from SEQ ID NO: 4, 14, 24, 34, and 44; a CDR2 comprising an amino acid sequence selected from SEQ ID NO: 5, 15, 25, 35, and 45; and a CDR3 comprising an amino acid sequence selected from SEQ ID NO: 6, 16, 26, 36, and 46.

Embodiment 55. The cell of embodiment 54, wherein the VH comprises an amino acid sequence selected from SEQ ID NO: 7, 17, 27, 37 and 47.

Embodiment 56. The cell of either embodiment 54 or 55, wherein the VL comprises an amino acid sequence selected from SEQ ID NO: 8, 18, 28, 38, and 48.

Embodiment 57. The cell of any one of embodiments 54 to 56, wherein the CLDN18.2 specific antigen binding domain comprises the sequence as set forth in SEQ ID NO: 52.

Embodiment 58. The cell of any one of embodiments 54 to 57, wherein the cell further comprises an armoring molecule.

Embodiment 59. The cell of embodiment 58, wherein the armoring molecule is selected from a dominant-negative TGFβ receptor type II, IL-7, IL-12, IL-15, IL-18, a hybrid IL-4/IL-7 receptor, a hybrid IL-7/IL-2 receptor, and HIF1α dominant-negative.

Embodiment 60. The cell of embodiment 59, wherein the armoring molecule comprises a dominant-negative TGFβ receptor type II (dnTGFβRII).

Embodiment 61. The cell of either embodiment 59 or 60, wherein the armoring molecule comprises an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO:54.

Embodiment 62. The cell of any one of embodiments 59 to 61, wherein the dominant negative TGFβ receptor type II comprises the sequence of SEQ ID NO:54.

Embodiment 63. The cell of any one of embodiments 52-62, wherein the cell is selected from a T cell, a Natural Killer (NK) cell, a cytotoxic T lymphocyte (CTL), a tumor infiltrating lymphocyte, and a regulatory T cell.

Embodiment 64. The cell of embodiment 63, wherein the cell exhibits an anti-tumor immunity upon contacting a tumor cell expressing CLDN18.2.

Embodiment 65. A method of treating cancer, comprising:
administering to a subject in need thereof an effective amount of a cell comprising an anti-CLDN18.2 chimeric antigen receptor (CAR) comprising an antigen binding domain, wherein the antigen binding domain comprises an antibody, Fab, or an scFv comprising a heavy chain variable region (VH) and a light chain variable region (VL),
wherein the VH comprises a CDR1 comprising an amino acid sequence selected from SEQ ID NO: 1, 11, 21, 31, and 41; a CDR2 comprising an amino acid sequence selected from SEQ ID NO: 2, 12, 22, 32, and 42; and a CDR3 comprising an amino acid sequence selected from SEQ ID NO: 3 13, 23, 33, and 43; and
wherein the VL comprises a CDR1 comprising an amino acid sequence selected from SEQ ID NO: 4, 14, 24, 34, and 44; a CDR2 comprising an amino acid sequence selected from SEQ ID NO: 5, 15, 25, 35, and 45; and a CDR3 comprising an amino acid sequence selected from SEQ ID NO: 6, 16, 26, 36, and 46.

Embodiment 66. The method of embodiment 65 further comprising inhibiting tumor growth, inducing tumor regression, and/or prolonging survival of the subject.

Embodiment 67. The method of embodiment 65, wherein the cell is an autologous cell.

Embodiment 68. The method of embodiment 67, wherein the autologous cell is selected from a T cell, a Natural Killer (NK) cell, a cytotoxic T lymphocyte (CTL), a tumor infiltrating lymphocyte, and a regulatory T cell.

Embodiment 69. The method of any of embodiments 65-68, wherein the cancer is a solid tumor.

Embodiment 70. The method of embodiment 69, wherein the solid tumor is gastric cancer, gastroesophageal junction cancer (GEJ; e.g., distal oesophageal cancers, proximal gastric cancers and cancers of cardia), pancreatic cancer, breast cancer, colon cancer, liver cancer, head and neck cancer, bronchial cancer, biliary adenocarcinoma, ovarian cancer, hepatocellular carcinoma, or non-small-cell lung cancer.

Embodiment 71. The method of embodiment 70, wherein the solid tumor is pancreatic cancer.

Embodiment 72. An antibody or an antigen-binding portion thereof that specifically binds CLDN18.2, comprising a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3, wherein:
(a) the VH-CDR1 comprises an amino acid sequence selected from SEQ ID NOs: 1, 11, 21, 31, and 41;
(b) the VH-CDR2 comprises an amino acid sequence selected from SEQ ID NOs: 2, 12, 22, 32, and 42;
(c) the VH-CDR3 comprises an amino acid sequence selected from SEQ ID NOs: 3, 13, 23, 33, and 43;
(d) the VL-CDR1 comprises an amino acid sequence selected from SEQ ID NOs: 4, 14, 24, 34, and 44;
(e) the VL-CDR2 comprises an amino acid sequence selected from SEQ ID NOs: 5, 15, 25, 35, and 45; and
(f) the VL-CDR3 comprises an amino acid sequence selected from SEQ ID NOs: 6, 16, 26, 36, and 46.

Embodiment 73. The antibody or an antigen-binding portion thereof of embodiment 72, wherein:
(a) the VH-CDR1 comprises the amino acid sequence set forth in SEQ ID NO: 1, the VH-CDR2 comprises the amino acid sequence set forth in SEQ ID NO: 2, the VH-CDR3 comprises the amino acid sequence set forth in SEQ ID NO: 3, the VL-CDR1 comprises the amino acid sequence set forth in SEQ ID NO: 4, the VL-CDR2 comprises the amino acid sequence set forth in SEQ ID NO: 5, and the VL-CDR3 comprises the amino acid sequence set forth in SEQ ID NO: 6;
(b) the VH-CDR1 comprises the amino acid sequence set forth in SEQ ID NO: 11, the VH-CDR2 comprises the amino acid sequence set forth in SEQ ID NO: 12, the VH-CDR3 comprises the amino acid sequence set forth in SEQ ID NO: 13, the VL-CDR1 comprises the amino acid sequence set forth in SEQ ID NO: 14, the VL-CDR2 comprises the amino acid sequence set forth in SEQ ID NO: 15, and the VL-CDR3 comprises the amino acid sequence set forth in SEQ ID NO: 16;
(c) the VH-CDR1 comprises the amino acid sequence set forth in SEQ ID NO: 21, the VH-CDR2 comprises the amino acid sequence set forth in SEQ ID NO: 22, the VH-CDR3 comprises the amino acid sequence set forth in SEQ ID NO: 23, the VL-CDR1 comprises the amino acid sequence set forth in SEQ ID NO: 24, the VL-CDR2 comprises the amino acid sequence set forth in SEQ ID NO: 25, and the VL-CDR3 comprises the amino acid sequence set forth in SEQ ID NO: 26;
(d) the VH-CDR1 comprises the amino acid sequence set forth in SEQ ID NO: 31, the VH-CDR2 comprises the amino acid sequence set forth in SEQ ID NO: 32, the VH-CDR3 comprises the amino acid sequence set forth in SEQ ID NO: 33, the VL-CDR1 comprises the amino acid sequence set forth in SEQ ID NO: 34, the VL-CDR2 comprises the amino acid sequence set forth in SEQ ID NO: 35, and the VL-CDR3 comprises the amino acid sequence set forth in SEQ ID NO: 36; or
(e) the VH-CDR1 comprises the amino acid sequence set forth in SEQ ID NO: 41, the VH-CDR2 comprises the amino acid sequence set forth in SEQ ID NO: 42, the VH-CDR3 comprises the amino acid sequence set forth in SEQ ID NO: 43, the VL-CDR1 comprises the amino acid sequence set forth in SEQ ID NO: 44, the VL-CDR2 comprises the amino acid sequence set forth in SEQ ID NO: 45, and the VL-CDR3 comprises the amino acid sequence set forth in SEQ ID NO: 46.

Embodiment 74. The antibody or an antigen-binding portion thereof of embodiment 72, wherein the VH comprises an amino acid sequence having at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 7, 17, 27, 37, and 47.

Embodiment 75. The antibody or an antigen-binding portion thereof of any one of embodiments 72 to 74, wherein the VH comprises an amino acid sequence selected from SEQ ID NOs: 7, 17, 27, 37, and 47.

Embodiment 76. The antibody or an antigen-binding portion thereof of any one of embodiments 72 to 75, wherein the VL comprises an amino acid sequence having at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 8, 18, 28, 38, and 48.

Embodiment 77. The antibody or an antigen-binding portion thereof of any one of embodiments 72 to 75, wherein the VL comprises an amino acid sequence selected from SEQ ID NOs: 8, 18, 28, 38, and 48.

Embodiment 78. The antibody or an antigen-binding portion thereof of any one of embodiments 72 to 77, wherein:
(a) the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7, and the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8;
(b) the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17, and the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18;
(c) the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 27, and the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 28;
(d) the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 37, and the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 38; or
(e) the VH comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 47, and the VL comprises an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 48.

Embodiment 79. The antibody or an antigen-binding portion thereof of any one of embodiments 72 to 78, wherein:
(a) the VH comprises the amino acid sequence set forth in SEQ ID NO: 7, and the VL comprises the amino acid sequence set forth in SEQ ID NO: 8;
(b) the VH comprises the amino acid sequence set forth in SEQ ID NO: 17, and the VL comprises the amino acid sequence set forth in SEQ ID NO: 18;
(c) the VH comprises the amino acid sequence set forth in SEQ ID NO: 27, and the VL comprises the amino acid sequence set forth in SEQ ID NO: 28;
(d) the VH comprises the amino acid sequence set forth in SEQ ID NO: 37, and the VL comprises the amino acid sequence set forth in SEQ ID NO: 38; or
(e) the VH comprises the amino acid sequence set forth in SEQ ID NO: 47, and the VL comprises the amino acid sequence set forth in SEQ ID NO: 48.

Embodiment 80. A pharmaceutical composition comprising the isolated nucleic acid of any one of embodiments 1 to 26, the anti-CLDN18.2 CAR of any one of embodiments 27 to 50, the vector of embodiment 51, the cell of any one of embodiments 52 to 64, or the antibody or the antigen-binding portion thereof of any one of embodiments 72 to 79, and a pharmaceutically acceptable excipient.

Embodiment 81. A method of treating a disease or condition in a subject in need thereof, comprising administering to the subject the isolated nucleic acid of any one of embodiments 1 to 26, the anti-CLDN18.2 CAR of any one of embodiments 27 to 50, the vector of embodiment 51, the cell of any one of embodiments 52 to 64, the antibody or the antigen-binding portion thereof of any one of embodiments 72 to 79, or the pharmaceutical composition of embodiment 80.

Embodiment 82. The method of embodiment 81, wherein the disease or condition comprises a cancer.

Embodiment 83. A method of treating a cancer in a subject in need thereof, comprising administering to the subject the isolated nucleic acid of any one of embodiments 1 to 26, the anti-CLDN18.2 CAR of any one of embodiments 27 to 50, the vector of embodiment 51, the cell of any one of embodiments 52 to 64, the antibody or the antigen-binding portion thereof of any one of embodiments 72 to 79, or the pharmaceutical composition of embodiment 80.

Embodiment 84. The method of either embodiment 82 or 83, wherein the cancer is gastric cancer, gastroesophageal junction cancer (GEJ; e.g., distal oesophageal cancers, proximal gastric cancers and cancers of cardia), pancreatic cancer, breast cancer, colon cancer, liver cancer, head and neck cancer, bronchial cancer, biliary adenocarcinoma, ovarian cancer, hepatocellular carcinoma, or non-small-cell lung cancer.

Embodiment 85. Use of the isolated nucleic acid of any one of embodiments 1 to 26, the anti-CLDN18.2 CAR of any one of embodiments 27 to 50, the vector of embodiment 51, the cell of any one of embodiments 52 to 64, the antibody or the antigen-binding portion thereof of any one of embodiments 72 to 79, or the pharmaceutical composition of embodiment 80 in the treatment of a disease or condition in a subject in need thereof.

Embodiment 86. The use of embodiment 85, wherein the disease or condition comprises a cancer.

Embodiment 87. Use of the isolated nucleic acid of any one of embodiments 1 to 26, the anti-CLDN18.2 CAR of any one of embodiments 27 to 50, the vector of embodiment 51, the cell of any one of embodiments 52 to 60, the antibody or the antigen-binding portion thereof of any one of embodiments 72 to 79, or the pharmaceutical composition of embodiment 80 in the treatment of a cancer in a subject in need thereof.

Embodiment 88. The use of either embodiment 86 or 87, wherein the cancer is gastric cancer, gastroesophageal junction cancer (GEJ; e.g., distal oesophageal cancers, proximal gastric cancers and cancers of cardia), pancreatic cancer, breast cancer, colon cancer, liver cancer, head and neck cancer, bronchial cancer, biliary adenocarcinoma, ovarian cancer, hepatocellular carcinoma, or non-small-cell lung cancer.

Embodiment 89. A method of expanding a population of T cells comprising:
(a) isolating CD3⁺ T cells from a sample;
(b) culturing the CD3⁺ T cells in a culture media that comprises human interleukin 21 (IL-21);
(c) activating the CD3⁺ T cells;
(d) transducing the CD3⁺ T cells with a vector comprising a nucleic acid encoding a chimeric antigen receptor (CAR) that binds CLDN18.2 to produce CAR-T cells;
(e) culturing the CAR-T cells in a medium; and
(f) harvesting the CAR-T cells.

Embodiment 90. A method of manufacturing a T cell therapeutic comprising:
(a) obtaining a sample comprising a population of CD3⁺ T cells;
(b) culturing the CD3⁺ T cells in a culture media that comprises human interleukin 21 (IL-21);
(c) activating the CD3⁺ T cells;
(d) transducing the CD3⁺ T cells with a vector comprising a nucleic acid encoding a chimeric antigen receptor (CAR) that binds CLDN18.2 to produce CAR-T cells;
(e) culturing the CAR-T cells or T-cell Receptor (TCR) cells in a medium; and
(f) harvesting the CAR-T cells.

Embodiment 91. The method of either embodiment 89 or 90, wherein the population of CD3+ T cells is formed from an isolated population of CD4+ and CD8+ T cells.

Embodiment 92. The method of any one of embodiments 89 to 91, wherein the culture media further comprises human interleukin 2 (IL-2).

Embodiment 93. The method of any one of embodiments 89 to 92, wherein about from 1×106 to about 1×109 CD3+ T cells are cultured in step (b) in the culture media.

Embodiment 94. The method of any one of embodiments 89 to 93, wherein the sample is an enriched apheresis product collected via leukapheresis.

Embodiment 95. The method of any one of embodiments 89 to 94, wherein the CD3+ T cells in step (c) are cultured for about one day or about two days.

Embodiment 96. The method of any one of embodiments 89 to 95, wherein the CD3+ T cells in step (c) are activated with agonists of CD2, CD3, CD28, or any combination thereof.

Embodiment 97. The method of any one of embodiments 89 to 96, wherein the CD3+ T cells in step (c) are activated with magnetic microbeads.

Embodiment 98. The method of any one of embodiments 89 to 97, wherein the CD3+ T cells in step (c) are activated with an anti-CD3 antibody or CD3-binding fragment thereof, and an anti-CD28 antibody or a CD28-binding fragment thereof.

Embodiment 99. The method of embodiment 98, wherein the anti-CD3 antibody or CD3-binding fragment thereof, and the anti-CD28 antibody or a CD28-binding fragment thereof are coupled to a magnetic microbead.

Embodiment 100. The method of any one of embodiments 89 to 99, wherein the CAR-T cells are cultured in step (e) from about two to about ten days.

Embodiment 101. The method of any one of embodiments 89 to 99, wherein the CAR-T cells are cultured in step (e) from about four to about six days.

Embodiment 102. The method of embodiment 101, wherein the CAR-T cells are cultured in step (e) for about four days.

Embodiment 103. The method of embodiment 101, wherein the CAR-T cells are cultured in step (e) for about six days.

Embodiment 104. The method of any one of embodiments 92 to 103, wherein the concentration of human IL-21 is from about 0.01 U/mL to about 0.3 U/mL, and the concentration of human IL-2 is from about 5 IU/mL to about 100 IU/mL.

Embodiment 105. The method of any one of embodiments 89 to 104, wherein the concentration of human IL-21 is about 0.19 U/mL.

Embodiment 106. The method of embodiment 105, wherein the concentration of human IL-2 is about 40 IU/mL.

Embodiment 107. The method of any one of embodiments 89 to 106, wherein the CD3+ T cells are agitated during step (b).

Embodiment 108. A method of manufacturing a T cell therapeutic comprising: (a) isolating CD4+ and CD8+ T cells from a sample to form a population of CD3+ T cells; (b) culturing the CD3+ T cells in a culture media that comprises human interleukin 2 at a concentration of 40 IU/mL and human interleukin 21 at a concentration of 0.19 U/mL; (c) activating the CD3+ T cells with a magnetic bead comprising an anti-CD3 antibody or CD3-binding fragment thereof, and an anti-CD28 antibody or a CD28-binding fragment thereof; (d) transducing the CD3+ T cells with a vector comprising a nucleic acid encoding a chimeric antigen receptor (CAR) that binds CLDN18.2 to produce CAR-T cells; (e) culturing the CAR-T cells in a medium for about four days; and (f) harvesting the CAR-T cells.

Embodiment 109. The method of any one of embodiments 89 to 108, wherein the vector is a virus, a lentivirus, an adenovirus, a retrovirus, an adeno-associated virus (AAV), a transposon, a DNA vector, a mRNA, a lipid nanoparticle (LNP), or a CRISPR-Cas System.

Embodiment 110. The method of any one of embodiments 89 to 109, wherein the vector is a lentivirus.

Embodiment 111. The method of embodiment 110, wherein the lentivirus is added at a multiplicity of invention (MOI) of about 0.25 to about 20.

Embodiment 112. The method of embodiment 111, wherein the lentivirus is added at a MOI of about 1 to about 4.

Embodiment 113. The method of embodiment 111, wherein the lentivirus is added at a MOI of about 2, or about 4.

Embodiment 114. The method of any one of embodiments 89 to 113, wherein the cell culture media is increased in volume after step (d).

Embodiment 115. The method of embodiment 114, wherein the cell culture media is increased in volume at least about 6 fold.

Embodiment 116. The method of any one of embodiments 89 to 115, wherein the medium in step (e) is exchanged at least once per day.

Embodiment 117. The method of any one of embodiments 89 to 116, wherein the medium in step (e) is exchanged about every 12 hours.

Embodiment 118. The method of any one of embodiments 89 to 117, wherein the CAR-T cells are expanded from at least about 1 fold to about 5 fold during step (e).

Embodiment 119. The method of any one of embodiments 89 to 117, wherein the CAR-T cells are expanded from at least about 1 fold to about 3 fold during step (e).

Embodiment 120. The method of embodiment 119, wherein the CAR-T cells are expanded about 2 fold during step (e).

Embodiment 121. The method of embodiment 119, wherein the CAR-T cells are expanded about 3 fold during step (e).

Embodiment 122. The method of any one of embodiments 89 to 121, wherein the CAR that binds CLDN18.2 comprises an antigen-binding domain comprising:
(a) a VH-CDR1 comprises the amino acid sequence set forth in SEQ ID NO: 1, a VH-CDR2 comprises the amino acid sequence set forth in SEQ ID NO: 2, a VH-CDR3 comprises the amino acid sequence set forth in SEQ ID NO: 3, a VL-CDR1 comprises the amino acid sequence set forth in SEQ ID NO: 4, a VL-CDR2 comprises the amino acid sequence set forth in SEQ ID NO: 5, and a VL-CDR3 comprises the amino acid sequence set forth in SEQ ID NO: 6;
(b) a VH-CDR1 comprises the amino acid sequence set forth in SEQ ID NO: 11, a VH-CDR2 comprises the amino acid sequence set forth in SEQ ID NO: 12, a VH-CDR3 comprises the amino acid sequence set forth in SEQ ID NO: 13, a VL-CDR1 comprises the amino acid sequence set forth in SEQ ID NO: 14, a VL-CDR2 comprises the amino acid sequence set forth in SEQ ID NO: 15, and a VL-CDR3 comprises the amino acid sequence set forth in SEQ ID NO: 16;
(c) a VH-CDR1 comprises the amino acid sequence set forth in SEQ ID NO: 21, a VH-CDR2 comprises the amino acid sequence set forth in SEQ ID NO: 22, a VH-CDR3 comprises the amino acid sequence set forth in SEQ ID NO: 23, a VL-CDR1 comprises the amino acid sequence set forth in SEQ ID NO: 24, a VL-CDR2 comprises the amino acid sequence set forth in SEQ ID NO: 25, and a VL-CDR3 comprises the amino acid sequence set forth in SEQ ID NO: 26;
(d) a VH-CDR1 comprises the amino acid sequence set forth in SEQ ID NO: 31, a VH-CDR2 comprises the amino acid sequence set forth in SEQ ID NO: 32, a VH-CDR3 comprises the amino acid sequence set forth in SEQ ID NO: 33, a VL-CDR1 comprises the amino acid sequence set forth in SEQ ID NO: 34, a VL-CDR2 comprises the amino acid sequence set forth in SEQ ID NO: 35, and a VL-CDR3 comprises the amino acid sequence set forth in SEQ ID NO: 36; or
(e) a VH-CDR1 comprises the amino acid sequence set forth in SEQ ID NO: 41, a VH-CDR2 comprises the amino acid sequence set forth in SEQ ID NO: 42, a VH-CDR3 comprises the amino acid sequence set forth in SEQ ID NO: 43, a VL-CDR1 comprises the amino acid sequence set forth in SEQ ID NO: 44, a VL-CDR2 comprises the amino acid sequence set forth in SEQ ID NO: 45, and a VL-CDR3 comprises the amino acid sequence set forth in SEQ ID NO: 46.

Embodiment 123. The method of embodiment 122, wherein the CAR that binds CLDN18.2 comprises a VH comprising an amino acid sequence having at least about 75%, at least about 80%, at least about 85%, at least about 90%, at least about 95%, at least about 96%, at least about 97%, at least about 98%, or at least about 99% sequence identity to the amino acid sequence selected from SEQ ID NOs: 7, 17, 27, 37, and 47.

Embodiment 124. The method of embodiment 122, wherein the CAR that binds CLDN18.2 comprises a VH comprising an amino acid sequence selected from SEQ ID NOs: 7, 17, 27, 37, and 47.

Embodiment 125. The method of any one of embodiments 122 to 124, wherein the CAR that binds CLDN18.2 comprises a VL comprising an amino acid sequence having at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 8, 18, 28, 38, and 48.

Embodiment 126. The method of embodiment 125, wherein the CAR that binds CLDN18.2 comprises a VL comprising an amino acid sequence selected from SEQ ID NOs: 8, 18, 28, 38, and 48.

Embodiment 127. The method of any one of embodiments 122 to 126, wherein the CAR that binds CLDN18.2 comprises:
(a) a VH comprising an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7, and a VL comprising an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8;
(b) a VH comprising an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17, and a VL comprising an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18;
(c) a VH comprising an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 27, and a VL comprising an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 28;
(d) a VH comprising an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 37, and a VL comprising an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 38; or
(e) a VH comprising an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 47, and a VL comprising an amino acid sequence having at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 48.

Embodiment 128. The method of embodiment 127, wherein the CAR that binds CLDN18.2 comprises:
(a) a VH comprising the amino acid sequence set forth in SEQ ID NO: 7, and a VL comprising the amino acid sequence set forth in SEQ ID NO: 8;
(b) a VH comprising the amino acid sequence set forth in SEQ ID NO: 17, and a VL comprising the amino acid sequence set forth in SEQ ID NO: 18;
(c) a VH comprising the amino acid sequence set forth in SEQ ID NO: 27, and a VL comprising the amino acid sequence set forth in SEQ ID NO: 28;
(d) a VH comprising the amino acid sequence set forth in SEQ ID NO: 37, and a VL comprising the amino acid sequence set forth in SEQ ID NO: 38; or
(e) a VH comprising the amino acid sequence set forth in SEQ ID NO: 47, and a VL comprising the amino acid sequence set forth in SEQ ID NO: 48.

Embodiment 129. The method of any one of embodiments 89 to 125, wherein the CAR that binds CLDN18.2 comprises the sequence as set forth in SEQ ID NO: 52.

Embodiment 130. The method of any one of embodiments 89 to 129, wherein the nucleic acid encoding the CAR that binds CLDN18.2 further comprises an armoring domain comprising a nucleic acid encoding an armoring molecule, optionally wherein the armoring domain is located at the 3′ end of the nucleic acid encoding the CAR or at the 5′ end of the nucleic acid encoding the CAR.

Embodiment 131. The method of any one of embodiments 89 to 129, wherein the CAR-T cells comprise an armoring molecule.

Embodiment 132. The method of either embodiment 130 or 131, wherein the armoring molecule is selected from a dominant-negative TGFβ receptor type II, IL-7, IL-12, IL-15, IL-18, a hybrid IL-4/IL-7 receptor, a hybrid IL-7/IL-2 receptor, and HIF1α dominant-negative.

Embodiment 133. The method of any one of embodiments 130 to 132, wherein the armoring molecule comprises a dominant-negative TGFβ receptor type II (dnTGFβRII).

Embodiment 134. The method of any one of embodiments 130 to 133, wherein the armoring molecule comprises an amino acid sequence having at least about 75%, at least about 80%, at least about 85%, at least about 90%, at least about 95%, at least about 96%, at least about 97%, at least about 98%, or at least about 99% sequence identity to the amino acid sequence of SEQ ID NO:54.

Embodiment 135. The method of any one of embodiments 132 to 134, wherein the dominant negative TGFβ receptor type II comprises the sequence of SEQ ID NO:54.

Embodiment 136. The method of any one of embodiments 89 to 113530, wherein the CAR-T cells are formulated in an isotonic solution.

Embodiment 137. The method of embodiment 136, wherein the isotonic solution comprises plasmalyte containing human serum albumin.

Embodiment 138. The method of either embodiment 136 or claim 137, wherein the isotonic solution contains between about 1×106 and about 1×109 CAR-T cells.

Embodiment 139. The method of embodiment 138, wherein the isotonic solution contains about 3.4×106 CAR-T cells.

Embodiment 140. The method of any one of embodiments 89 to 139, wherein the CAR-T cells are a mixture of TCM and TSCM cells.

Embodiment 141. The method of embodiment 140, wherein from about 15% to about 50% of the CAR-T cells are TSCM cells and express CD45RA, CCR7 and CD27, and do not express CD45RO.

Embodiment 142. The method of embodiment 141, wherein about 20% to about 30% of the CAR-T cells are TSCM cells and express CD45RA, CCR7 and CD27, and do not express CD45RO.

Embodiment 143. The method of any one of embodiments 89 to 142, wherein more than 50% of the CAR-T cells express a chimeric antigen receptor.

Embodiment 144. The method of embodiment 143, wherein from about 40% to about 60% of the CAR-T cells express a chimeric antigen receptor.

Embodiment 145. The method of any one of embodiments 89 to 144, wherein more than 50% of the CAR-T cells express CD8.

Embodiment 146. The method of embodiment 145, wherein from about 40% to about 60% of the CAR-T cells express CD8.

It is to be understood that the particular aspects of the specification are described herein are not limited to specific embodiments presented, and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

### EXAMPLES

The Examples that follow are illustrative of specific embodiments of the disclosure, and various uses thereof. They are set forth for explanatory purposes only and should not be construed as limiting the scope of the disclosure in any way.

### BACKGROUND

Chimeric antigen receptor T cell therapies have exhibited exceptional anti-tumor activities for so-called liquid tumors or cancers that develop in the blood, bone marrow or lymph nodes. However, CAR-T therapies with potent activities for solid tumors has been elusive. The reasons for lack of translation to solid tumors is multifold, but can be attributed to a few factors. First of which is selection of an antigen which has limited normal tissue expression so as to prevent so called “on-target, off-tumor toxicities”. As such, one of the first aspects critical to a successful CAR-T is selection of the tumor associated antigen, ideally with increased expression on tumor cells and limited expression or restricted access in normal tissues. CLDN18 is a well-characterized four transmembrane protein involved in the formation of tight junctions and maintenance of cell barrier function and cellular polarity. Two different isoforms of CLDN18, CLDN18.1 and CLDN18.2, have been identified, each with distinct normal tissue expression patterns with most normal tissue expression of CLDN18.2 being found in the differentiated cells of the gastric mucosa. Elevated levels of CLDN18.2 have been observed in a high proportion of pancreatic, gastric and esophageal adenocarcinomas, with some expression though lower prevalence in other cancer indications including colorectal, ovarian and biliary tree cancers. Expression of CLDN18.2 is maintained across animal species, with homology being 99% with cynomolgus monkey, 89% with murine and 90% with rat CLDN18.2.

Another challenge to translate the success of CAR-Ts in hematologic malignancies to solid tumors is the highly immunosuppressive tumor microenvironment often faced by the CAR-Ts once they are able to persist and infiltrate to the tumor site. This includes presence of suppressive immune cells in the TME, such as Tregs, myeloid derived suppressor cells (MDSCs), and tumor associated macrophages (TAMs), which promote tumor cell proliferation, metastasis, and secretion of inhibitory cytokines, such as IL-4, IL-10, and TGFβ, which can shut down T cell function.

An additional hurdle to reach effective responses in solid tumors is to develop CAR-T cells which can continue to expand and persist post reinfusion to prevent potential tumor regrowth or relapse. It has been established that selection or generation of CAR-T cells with a less differentiated or T_{SCM} phenotype can endow the CAR-T product with enhanced self-renewal and proliferative capacity which may lead to enhanced persistence and a more robust anti-tumor response (Gattinoni, Nat Med 2011; 17:1290-7). Generation of a less differentiated CAR-T can be accomplished by multiple approaches, including shortened and optimized manufacturing protocols. To deliver CAR-Ts to patients, T cells are isolated from patient blood then genetically engineered and manufactured ex vivo, going through multiple doublings and expanded until CAR-T numbers are reached to dose or reinfuse to the patient. Optimization of the manufacturing strategy to generate an infusion product enriched in these less differentiated cells is a means to generate a more persistent CAR-T cell with potential for enhanced anti-tumor activity.

### Materials and Methods:

### Cell Lines:

All cells were cultured in medium as per supplier recommendations and maintained in tissue culture flasks at 37° C. in a humidified atmosphere at 5% CO₂. Aspc1, BxPC3, HEK293 and NCI-N87 were obtained from the American Tissue Culture Collection (ATCC, Manassas, VA). NUGC4 was obtained from Riken BioResource Research Center (Ibaraki, Japan). SNU-601 was obtained from the Korean Cell Line Bank (Seoul, Korea). The PaTu 8988s cell line (“unsorted”), which endogenously expresses CLDN18.2, was obtained from the DSMZ collection (Braunschweig, Germany).

### Lentivirus Prep

To generate cell lines expressing CLDN18 variants, DNA encoding: human CLDN18.2 (Uniprot: P56856-2), human CLDN18.1 (Uniprot: P56856-1), human CLDN18.2 M149L, human CLDN18.2 Q29M, human CLDN18.2 N37D, human CLDN18.2 A42S, human CLDN18.2 N45Q, human CLDN18.2 Q47E, human CLDN18.2 E56Q, human CLDN18.2 G65P, human CLDN18.2 L69I, cynomolgous monkey CLDN18.2 (Uniprot: A0A2K5VV62), cynomolgous monkey CLDN18.1 (Uniprot: A0A2K5VVB4), rat CLDN18.2 (Uniprot: Q5I0E5), rat CLDN18.1 (Uniprot: P56857-3), mouse CLDN18.2 (Uniprot: P56857-3), and mouse CLDN18.1 (Uniprot: P56857) were obtained from Integrated DNA Technologies (Coralville, IA), and transferred into the lentiviral pCDH-CMV-MCS-EF1-Puro vector from System Biosciences (Palo Alto, CA). The pCDH-CMV-MCS-EF1-Puro vector expresses the gene incorporated into the multiple cloning site (MCS) and the puromycin resistance gene used for antibiotic selection. In addition, DNA encoding human, cynomolgous monkey, rat and mouse CLDN18.2 were transferred into a modified lentiviral pCDH1-CMV-MCS-EF1-Puro-T2A-GFP vector. The modified pCDH1-CMV-MCS-EF1-Puro-T2A-GFP vector expresses the gene incorporated into the MCS, the puromycin resistance gene for antibiotic selection and GFP, which proved useful for high throughput screening.

To generate lentiviruses, lentiviral vectors were co-transfected with pPACKH1 (System Biosciences, cat no LV500A-1) into suspension HEK293 cells and incubated overnight at 37° C., 8% CO₂ and 125 RPM. On day 1 post-transfection, cultures containing transfected cells were centrifuged for 5 minutes at 2500 RPM. Culture supernatants were discarded and pelleted cells were resuspended in 30 mL fresh FreeStyle 293 media and incubated at 37° C., 8% CO₂ and 125 RPM. On day 2 post-transfection, suspension HEK293 cultures were transferred to a 50 mL conical tube and centrifuged for 5 minutes at 2500 RPM. Culture supernatants containing lentivirus were filtered and centrifuged at 100,000×g for 2 hours. Pelleted lentivirus was resuspended in 600 μL Opti-MEM (ThermoFisher Scientific cat no 31985062), aliquoted in cryovials, and stored at −80° C.

To generate suspension HEK293 cells expressing CLDN18 variants, suspension HEK293 were diluted to 4E5 cells/mL in 15 mL and transduced with 50 μL 50× concentrated lentivirus. Cells were grown at 37° C., 8% CO₂ and 125 RPM for 3 days before being selected with 2 μg/mL puromycin, expanded and banked. Aspc1, HEK293, NCI-N87, BxPC3, and NUGC4 were similarly transduced with lentivirus and selected with puromycin although media and culture requirements were different.

### Example 1. Development and Characterization of Anti-CLDN18.2 Antibodies and/or Antigen Binding Fragments Thereof

### Cell-Based Phage Selections for Isolation of CLDN18.2-Specific Leads

CLDN18.2-reactive scFv leads were generated through cell-based phage selections. Engineered HEK293 cells expressing human CLDN18.2 (Clone D2=150,000 receptors/cell) were used as source antigen for selection of CLDN18.2-reactive phage from the recombinant framework (REF) single-chain variable fragment (scFv) phage library. The REF phage library is a naïve synthetic VH-VL scFv library that is based on the IGHV1-69*01 and IGLV1-44*01 germlines; CDR H1-2 and CDR L1-2 contain wholly germline sequences, where the library diversity (1×10⁹) results from 9 randomized amino acids in CDR H3 (ARXXXXXXXXDX; SEQ ID NO:57) and 5 randomized amino acids in CDR L3 (AAWDXXXXXVV; SEQ ID NO:58).

In brief, HEK293 cells expressing human CLDN18.2 (Clone D2) and an aliquot of the REF phage library containing 10 12 phage were blocked in DMEM supplemented with 10% FBS for 1 hour at room temperature with gentle shaking, blocked cells and library were then incubated for 1 hour with gentle shaking, cells and bound phage were washed extensively with PBS, and phage were recovered by addition of triethylamine (TEA). Recovered phage were used to infect exponentially growing TG1 for 1 hour at 37° C. An aliquot of the infected TG1 culture was used to titrate the selection output and the remaining infected TG1 culture was centrifuged at 3000 RPM for 10 minutes. The culture supernatant was discarded and the pellet representing infected TG1 was resuspended in 500 μL 2×TYCG (2×YT media containing 100 μg/mL carbenicillin and 2% glucose), spread on 2×TYCG agar bioassay plates, and incubated overnight at 30° C. Bioassay plates containing carbenicillin-resistant TG1 were scraped and transferred to a 50 mL polypropylene tube containing 10 mL 2×TYCG. Aliquots of the selection output were prepared for long-term storage, DNA isolation, and phage rescue. For long-term storage, 1200 μL selection output was transferred to cryovials containing 600 μL 50% (v/v) glycerol and stored at −80° C. For DNA isolation, phagemid DNA was isolated from selection output using the Plasmid Plus Maxi Kit (Qiagen, Cat no. 12963) according to the manufacturer's protocol. Isolated DNA was stored at −20° C.

For phage rescue, 50-100 μL of the selection output was used to inoculate 50 mL 2×YTCG and grown at 37° C. and 250 RPM until an OD600 of 0.5 was reached. A portion of the culture (25 mL) was transferred to a 50 mL polypropylene tube, super-infected with M13KO7 helper phage (MOI>10) for 1 hour at 37° C. (30 minutes stationary and 30 minutes shaken at 150 RPM). Helper phage-infected selection output was then centrifuged for 10 minutes at 3000 RPM. The cellular supernatant containing helper phage was discarded and the cellular pellet was resuspended in 25 mL 2×TYCK (2×TY media containing 100 μg/mL carbenicillin and 30 μg/mL kanamycin) and grown overnight at 25° C. and 250 RPM in a 250 mL Erlenmeyer culture flask. Overnight cultures were transferred to a 50 mL polypropylene tube and centrifuged at 4750 RPM for 15 minutes at 4° C. Cellular supernatants were transferred to a fresh 50 mL polypropylene tube and centrifuged at 8000 RPM for 25 minutes at 4° C. The supernatant containing amplified phage was then transferred to a fresh 50 mL polypropylene tube containing 6 mL PEG/NaCl, mixed gently, and incubated for 1 hour on ice. PEG-precipitated phage was harvested by centrifugation for 25 minutes at 8000 RPM and 4° C. The supernatant was discarded and the phage pellet was resuspended in 1 mL PBS-LT (phosphate-buffered saline supplemented with 0.01% (v/v) Tween 20) and transferred to a 1.5 mL Eppendorf tube. The phage suspension was then centrifuged for 10 minutes at 24,000×g and 4° C. to remove contaminating bacteria. Eight hundred μL of the supernatant was transferred to a fresh 1.5 mL Eppendorf tube containing 200 μL PEG-NaCl and incubated for 15 minutes on ice. Twice PEG-precipitated phage was then centrifuged at 4,000×g for 10 minutes at 4° C. The supernatant was discarded and the phage pellet was resuspended in 400 μL PBS-LT and transferred to a fresh 1.5 mL Eppendorf tube. The phage suspension was then centrifuged at 24,000×g for 10 minutes at 4° C. Pure and soluble phage was then transferred to a fresh 1.5 mL Eppendorf tube, titer determined and stored at 4° C.

In all, three rounds of phage selections were performed on CLDN18.2-expressing HEK293 cells, where phage selections exhibited round-to-round enrichment of CLDN18.2-reactive leads. Round 2 selection outputs exhibited favorable specificity and diversity profiles and were used as the basis for a large-scale screen.

### Screening Leads for CLDN18.2 Specificity

Candidate scFvs from phage selections were converted to scFv-Fc format for screening by flow cytometry. CLDN18.2 isoform reactivity and specificity were assessed by evaluating the binding candidate scFv-Fcs to HEK293 expressing CLDN18.2 and CLDN18.1 derived from human, rat and mouse and PaTu 8988s. PaTu 8988s is a pancreatic cancer-derived cell line that endogenously expresses human CLDN18.2.

Briefly, bulk phagemid DNA from selection outputs were digested overnight with NotI (New England BioLabs, R3189) and SfiI (New England BioLabs, R0123) at 37° C. for 6 hours and 50° C. for 6 hours. DNA fragments representing the scFv-encoding sequence were gel-purified using the QIAquick Gel Extraction Kit (Qiagen, cat no 28706), ligated into NotI- and SfiI-digested pSpliceV4 using T4 ligase (New England BioLabs, M0202), and transformed into One Shot TOP10 cells (New England BioLabs, C3019). The pSpliceV4 vector encodes a mammalian signal sequence, multiple cloning site, and human IgG Fc domain. Transformants were grown for 1.5 hours at 250 RPM and 37° C., plated on 2×TYCG agar bioassay plates and incubated overnight at 37° C.

Eighty-eight bacterial colonies representing individual transformants were selected by ClonePix and transferred to each 96 deep well plate containing 1.2 mL 2×TY media supplemented with 100 μg/mL carbenicillin; the remaining wells were left empty and used for positive and negative screening controls. Inoculated culture plates were sealed with two breathable membranes and grown overnight at 800 RPM and 37° C. Fifty μL overnight culture were transferred to a 96 well round bottom plates (VWR cat no 73520-474) containing 50 μL 50% (v/v) glycerol and stored at −80° C. DNA was isolated from the remaining bacterial culture using the NucleoSpin 96 Plasmid Kit (Macherey-Nagel, cat no 740625.4) according to the manufacturer's protocol except that DNA was eluted in 120 μL nuclease-free water after a 5-10 minute incubation at room temperature. The resulting DNA (35-45 ng/μL) in 96 well round bottom plates was stored at −20° C.

Transient transfections were used to generate candidate scFv-Fcs. Briefly, suspension HEK293 were split to a density of 0.7E6 cells/mL the day before transfection. On the day of transfection, 0.53 μL 293Fectin (ThermoFisher Scientific cat no 12347019), 24 μL Opti-MEM and 10 μL pSpliceV4 DNA (350-450 ng) were added to each well for transfection. 293Fectin, Opti-MEM and DNA were incubated for 20-25 minutes at room temperature before addition of 350 μL suspension HEK293 culture. Plates containing transfected cells were sealed with two breathable membranes and incubated at 37° C., 8% CO₂ and 350 RPM. After 3 days post-transfection, each well of culture was supplemented with 150 μL FreeStyle 293 expression medium and cultured for an additional 3 days. On day 6 post-transfection, cultures containing candidate scFv-Fcs were filtered using 96 well filters (Millipore, cat no MSHVS4510) and a vacuum apparatus. Clarified transfection supernatants were quantified and immediately tested in binding experiments by flow cytometry measurement.

For screening on suspension HEK293 cells, CLDN18.2 and CLDN18.1 binding and specificity were simultaneously assessed by flow cytometry. Briefly, 25,000 suspension HEK293 expressing human CLDN18.2 and green fluorescent protein (GFP) and 25,000 suspension HEK293 expressing human CLDN18.1 were washed and resuspended in 50 μL FACS buffer (PBS, pH 7.2 supplemented with 2% FBS, 2 mM EDTA and 0.1% sodium azide). Twenty-five μL FACS buffer and 25 μL clarified transfection supernatants were added to the corresponding wells. Dilute candidate scFv-Fcs were incubated with the mixed cell suspension on ice for 30 minutes. Cell-bound scFv-Fc were washed extensively with ice-cold FACS buffer and then stained with Alexa Fluor® 647 AffiniPure F(ab′)₂ Fragment Goat Anti-Human IgG (Jackson ImmunoResearch cat no 109-606-98) for 30 minutes on ice. Cells were then washed extensively with ice-cold FACS buffer and stained with FACS buffer supplemented with DAPI (ThermoFisher Scientific cat no 62248) for 10 minutes on ice. Positive and negative control for CLDN18.2 binding were included in all screening assessments; 5 μg/mL positive control CLDN18.2 antibody was used as positive control and a secondary only was used as negative control. Candidate scFv-Fc binding to cells was assessed on an IntelliCyt iQue (IntelliCyt). Cell populations were first gated for live/dead cells, and then gated based on GFP+/GFP−. ScFv-Fcs that exhibited CLDN18.2+ cell binding (GFP+) but not CLDN18.1+ cell binding (GFP−) were deemed potential leads and further characterized.

Candidate scFv-Fc binding specificity for rat CLDN18.2, but not rat CLDN18.1, and separately mouse CLDN18.2, but not mouse CLDN18.1 were assessed by flow cytometry in a manner similar to that accomplished for human CLDN18.2 and CLDN18.1. Cynomolgous monkey CLDN18.2 shares identical sequences in relevant extracellular domains to human CLDN18.2; therefore, screening cynomolgous monkey CLDN18.2 was not prioritized.

Instead, binding of candidate scFv-Fcs to PaTu 8988s, a pancreatic cancer cell line that endogenously expresses human CLDN18.2, was used to confirm CLDN18.2 binding capacity. In brief, PaTu 8988s cells were stained with clarified scFv-Fc supernatants, washed with FACS buffer, stained with Alexa Fluor® 647 AffiniPure F(ab′)₂ Fragment Goat Anti-Human IgG, washed with FACS buffer and stained with FACS buffer supplemented with DAPI. Candidate scFv-Fc binding to cells was assessed on an IntelliCyt iQue with gates for live/dead cells.

In all, 444 potential leads were identified from 2640 candidate scFv-Fcs screened. Of the 444 potential leads, 442 exhibited strong binding to PaTu 8988s (CLDN18.2+) cells. Of the 442 potential leads, 359 showed strong binding to rat CLDN18.2 but not rat CLDN18.1; however, of the 442 potential leads, only one showed strong binding to mouse CLDN18.2 but not mouse CLDN18.1.

### DNA Sequencing of CLDN18.2-Specific Leads

DNA sequences encoding all 442 CLDN18.2-specific scFvs were recovered from pSpliceV4 DNA preparations using pSpliceFwd (5′-CAGCTATGACCATGATTACGAATTT-3′; SEQ ID NO:59) and pMcoFcRev (5′-CTGATCATCAGGGTGTCCTTGG-3′; SEQ ID NO:60) by EuroFins (USA). Results were analyzed using DNASTAR software (Madison, WI) and identified 218 unique CLDN18.2-specific leads.

### Affinity and Cross-Reactivity Assessments of Leads in IgG1 Format

Prospective CLDN18.2-specific leads were converted to IgG1 format for affinity and cross-reactivity assessments. Due in large part to the restricted randomization inherent to the REF library, many of the CLDN18.2-specific sequences can be categorized as novel VH and VL, common VH and novel VL, variant VH and novel VL, or variant VH and common VL. Therefore, prioritized leads demonstrated: (1) high binding capacity to PaTu 8988s, (2) high selectivity for CLDN18.2 in human, rat, and mouse assessments; and (3) unique CDR H3 sequences with the goal of identifying antibodies with unique properties.

DNA encoding VH and VL of prospective CLDN18.2-specific leads were amplified by PCR and assembled into appropriately digested pOE-IgG1 (λLC) using NEBuilder HiFi DNA Assembly Master Mix (New England BioLabs, cat no E2621). Briefly, 100 μg PCR product, 50 μg digested pOE-IgG1 (λLC), 10 μL NEBuilder HiFi DNA Assembly Master Mix in 20 μL reaction in a 200 μL PCR tube. The assembly mix was briefly centrifuged and incubated at 50° C. for 1 hour. Following the incubation, 1 μl of the assembly products was transformed into One Shot TOP10 cells (New England BioLabs, C3019). Transformants were grown for 1 hour at 250 RPM and 37° C., plated on 2×TYC agar plates and incubated overnight at 37° C. Carbenicillin-resistant colonies were grown in 2×YTC media overnight at 37° C. DNA was isolated from inoculated cultures using QIAprep Spin Miniprep Kit (Qiagen, cat no 27106). DNA were sequenced using P130s FOR (5′-CCGTCGCCGCCACCATGGAC-3′; SEQ ID NO:61), P219 REV (5′-CTAGAAGGCACAGTCGAGGC-3′; SEQ ID NO:62), Signal pep intron FOR (5′-GGAGCTGTATCATCCTCTTC-3′; SEQ ID NO:63), and P220 REV (5′-GAGATGCTACTGGGGCAACGG-3′; SEQ ID NO:64). Sequence-verified expression constructs were used in transient transfections.

Chinese hamster ovary-derived G22 cells were used for transient transfections. Briefly, pOE-IgG1 (λLC) vectors containing prospective CLDN18.2-specific VH and VL domains were transfected into G22 cells using LIPOFECTAMINE™. Transfected G22 were fed using proprietary feed at 3 and 7 days post-transfection. Transfection supernatants were harvested on day 10 post-transfection, filtered and purified by MabSelect SuRE affinity and size-exclusion chromatography. Purified antibodies were pure and free of aggregates (>98.0% monomer).

Purified CLDN18.2 antibodies were then characterized for affinity and cross-reactivity by flow cytometry. Briefly. HEK293 cells expressing human, cynomolgous monkey, rat or mouse CLDN18.2 were resuspended in FACS Buffer, stained with varying antibody concentrations (0-533 nM), washed with FACS Buffer, stained with Alexa Fluor® 647 AffiniPure F(ab′)₂ Fragment Goat Anti-Human IgG, washed with FACS buffer and stained with FACS buffer supplemented with DAPI. Antibody binding to cells was assessed on a FACSymphony (BD) with gates for live/dead cells. Histograms of median fluorescence intensity (MFI) were used to calculate the geometric mean for each antibody at a given concentration (FlowJo, LLC). Geometric means were plotted in Prism (GraphPad) and used to calculated binding EC₅₀. Representative results are shown for 5 antibodies (ZP1I16_D05 IgG1, 008LY1_D04 IgG1, 08LYG_D08 IgG1, 008M0G_G03 IgG1, ZP1I18_B08 IgG1) and 1 negative control antibody (R347 IgG1) although 31 other prospective antibodies were also characterized (Table 1).

Results show that ZP1I16_D05 IgG1, 008LY1_D04 IgG1, 08LYG_D08 IgG1, 008M0G_G03 IgG1, ZP1I18_B08 IgG1 bind human and cynomolgous monkey CLDN18.2, ZP1I16_D05 IgG1, 08LYG_D08 IgG1, 008M0G_G03 IgG1, ZP1I18_B08 IgG1 bind rat CLDN18.2. And, only 08LYG_D08 IgG1 binds mouse CLDN18.2. Differences in human and rodent CLDN18.2 binding suggests that those antibodies possess conformational epitopes that encompass residues on both extracellular loop 1 (ECL1) and extracellular loop 2 (ECL2) as rodent CLDN18.2 have minor differences in ECL2 but identical ECL1. Moreover, all binding affinities and cross-reactivity assessments were consistent with screening results performed in scFv-Fc format.

### Epitope Assessment of Prospective CLDN18.2 Antibodies

Characteristics of the binding epitope, in particular membrane proximity, has been shown to critically influence the potency of CAR-T- and T cell engager-mediated cytolysis. Therefore, methods were developed to characterize the epitopes of prospective CLDN18.2 antibodies and prioritize leads for conversion to CAR-T format.

For this purpose, prospective antibody binding to wildtype and variants of human CLDN18.2 was characterized by flow cytometry. Variants were focused on determinants of human CLDN18.2 specificity by generating human CLDN18.2 variants that differ from wild-type by only one amino acid that appears in human CLDN18.1. In this way, we could differentiate antibody epitopes to specific regions and likely ensure the mutations would not significantly perturb overall CLDN18.2 architecture and surface expression. HEK293 expressing CLDN18.2 Q29M, CLDN18.2 N37D, CLDN18.2 A42S, CLDN18.2 N45Q, CLDN18.2 Q47E, CLDN18.2 E56Q, CLDN18.2 G65P, or CLDN18.2 L69I were generated for this purpose.

Analogous to the CLDN18.2 specificity screen, 25,000 HEK293 cells expressing wildtype CLDN18.2 were labeled with CellTrace™ CFSE Cell Proliferation Kit (C34554) and mixed with 25,000 unlabeled HEK293 expressing variant CLDN18.2 in 50 μL FACS buffer per well in a 96 well round bottom plate. For each antibody characterized, 8 wells were required to determine CLDN18.2 specificity. Cell mixtures were labeled with antibody in FACS buffer (10-20 μg/mL final concentration) for 30 minutes on ice, washed with FACS buffer, stained with Alexa Fluor® 647 AffiniPure F(ab′)₂ Fragment Goat Anti-Human IgG for 30 minutes on ice, washed with FACS buffer, and stained with FACS supplemented with DAPI for 10 minutes. Antibody binding to cells was assessed on a MACSQuant flow cytometer (MiltenyiBiotec) with gates for live/dead cells, FITC+ (CLDN18.2 wild-type) and FITC− (CLDN18.2 variant) cells.

For variants that do not participate in the binding epitope, the FITC+ and FITC− cell populations exhibit equivalent MFI in the APC channel. For variants that participate directly in the binding epitope, the FITC+ cell population exhibit strong binding in the APC channel, whereas the FITC− population exhibit little or no signal.

Thirty-six prospective CLDN18.2 antibodies were tested. Results are summarized in Table 2. Interestingly, a majority of the antibodies that were characterized possess epitopes that were sensitive to Q47E and L69I variants like 008LY1_D04 IgG1, which perhaps suggests that the REF library inherently favors this CLDN18.2 epitope. However, other epitopes were also identified. For example, ZP1I16_D05 IgG1 is sensitive to N45Q and Q47E; 08LYG_D08 IgG1 is sensitive to N45Q, Q47E, E56Q and E65P and possesses a membrane penetrant epitope; and, 008M0G_G03 and ZP1I18_B08 IgG1s are sensitive to N45Q, Q47E and L69I.

Surprisingly, ZP1I16_D05 IgG1 and ZP1I18_B08 IgG1 possess identical VH domains and only differ in 3 consecutive residues in CDR L3 but their epitope is considerably different.

### Assessment of Antibody Binding to HEK293 Expressing CLDN18.2 M149L

Binding of prospective antibodies to human CLDN18.2 M149L was also assessed as human CLDN18.2 M149L is a natural CLDN18.2 variant that exists at low level across CLDN18.2+ cancer patient population. In brief, HEK293 expressing human CLDN18.2 M149L were stained with a subset of prospective CLDN18.2 antibodies at 10 μg/mL in FACS buffer for 30 min, washed with FACS Buffer, stained with Alexa Fluor® 647 AffiniPure F(ab′)₂ Fragment Goat Anti-Human IgG, washed with FACS buffer, and stained with FACS buffer containing DAPI. Antibody binding to cells was assessed on a FAC Symphony (BD) with gates for live/dead cells. Histograms of mean fluorescence intensity (MFI) were used to assess binding to CLDN18.2 M149L. Results are shown in FIG. 3.

### Characterization of Internalization

In addition to prospective CLDN18.2 antibody affinity, cross-reactivity, and epitope characterizations, antibody internalization was also assessed. To this end, 36 prospective CLDN18.2 antibodies, positive control antibody and negative control antibody (R347 IgG1) were screened in a modified ZAP assay (Advanced Targeting Systems), where an anti-human-Fc Fab antibody (50 kDa) that had been conjugated to a potent DNA damaging cytotoxin was used to assess internalization via cell death. Briefly, on day 0, CLDN18.1 or CLDN18.2 expressing HEK293 cells were plated in tissue culture-treated 394 well plates (Corning 3765). The following day, a stock of media with a constant concentration of the toxin-conjugated Fab was prepared as solution 1 in plate 1, which alone showed minimal toxicity on CLDN18.2 targeting cells. A second plate was made with a dilution series of each test and control antibody. The stock solution one was mixed 1:1 with the media of each antibody in dilution curves such that the final concentration of anti-human-Fc Fab antibody was constant and the test anti-CLDN18.2 antibody was in series. This mixture was added to four replicate wells containing CLDN18.2+ cell lines at a 1:2 dilution (20 μl mixture plus 20 ul media in a 384 well plate) and plates were cultured for 6 days at 37° C. and 5% CO₂. At endpoint, viability was assessed using CellTiter-Glo® Luminescent Viability Assay (CTG, Promega, Madison, WI) according to the manufacturer's protocol and luminescence was read using an EnVision luminometer (Perkin Elmer, Waltham, MA). Cell viability was determined as follows: (average luminescence of treated samples/average luminescence of control samples)×100. IC₅₀ values were determined with GraphPad Prism software by using logistic non-linear regression analysis.

Out of the 36 prospective CLDN18.2 antibodies tested in the internalization assay, 35 exhibited an internalization phenotype, where ZP1I18_B08 IgG1 was the only antibody that did not exhibit internalization (FIG. 4A). In contrast, no internalization was observed in cells engineered to express CLDN18.1 (FIG. 4B).

### Example 2. CAR Conversion of Anti-CLDN18.2 Antibodies or Antigen Binding Fragments Thereof

Of the 36 prospective CLDN18.2 antibodies that were fully characterized, 5 CLDN18.2 antibodies were prioritized for conversion to CAR format and in vitro and in vivo assessments. Antibodies based on ZP1I16_D05, 008LY1_D04, 008LYG_D08, 008M0G_G03, ZP1I18_B08 exhibit high affinity and specificity for CLDN18.2, maintain desirable cross-reactivity profiles amongst relevant toxicology species, and possess unique conformational epitope and internalization characteristics.

To generate lentiviral expression vectors encoding CLDN18.2-reactive CARs, DNA encoding ZP1I16_D05, 008LY1_D04, 008LYG_D08, 008M0G_G03, ZP1I18_B08 scFv sequences were PCR amplified from pSpliceV4 and gel purified. ScFv encoding PCR products were then assembled into appropriately digested pESRC-CD33 leader-MCS-IgG4P-CD28 TM-4-1BB-CD3z-T2a-GFP or pESRC-CD33 leader-MCS-IgG4P-CD28 TM-4-1BB-CD3z-T2a-mCherry. These constructs contain sequences that encode the CD33 leader sequence, the IgG4 hinge with the S241P mutation (IgG4P), the transmembrane domain of CD28, the 4-1BB cytosolic domain, and a variant of the CD3z cytosolic domain, the self-cleaving T2a peptide, and either green florescent protein (GFP) or mCherry. In brief, 100 μg PCR product, 50 μg digested pESRC-MCS-IgG4P-CD28 TM-4-1BB-CD3ζ-T2a-GFP/mCherry, 10 μL NEBuilder HiFi DNA Assembly Master Mix in 20 μL reaction in a 200 μL PCR tube. The assembly mix was briefly centrifuged and incubated at 50° C. for 1 hour. Following the incubation, 1 μl of the assembly products was transformed into One Shot TOP10 cells (New England BioLabs, C3019). Transformants were grown for 1 hour at 250 RPM and 37° C., plated on 2×TYC agar plates and incubated overnight at 37° C. Carbenicillin-resistant colonies were grown in 2×YTC media overnight at 37° C. DNA was isolated from inoculated cultures using QIAprep Spin Miniprep Kit (Qiagen, cat no 27106). DNA was sequenced using EF1 FOR (5′-TTCGTTTTCTGTTCTGCGCCG-3′; SEQ ID NO:65) and 4-1BB REV (5′-TGTACAGCAGCTTCTTTCTGCC-3′; SEQ ID NO:66) for 4-1BB-containing vectors or EF1 FOR and CMS312s (5′-AGCCGTACATGAACTGAGGG-3′; SEQ ID NO:67). Sequence-verified constructs were used to generate lentivirus.

Alternative CAR formats were also explored, including alternate hinges (CD8, CD28), transmembrane domains (CD8), costimulatory domains (CD28), and CD3ζ domains (1XX, X1X, X2X ITAM variants).

### CAR-T Manufacture

For “traditional manufacture” CAR-T production and subsequent use in the in vitro and in vivo assays, purified human total T cells (CD4 and CD8) from healthy donors were activated with Dynabeads (Invitrogen) according to the manufacturers protocol and cells were grown in AIM-V medium containing 5% human AB serum (Valley Biomedical) and human IL-2 (300 IU/ml, Peprotech). After overnight activation, lentivirus was added to T cells at a M01=5 in addition to polybrene (1 ug/ml) and cells were centrifuged at 37° C., 2500 rpm for 2 hours. 72 hours post addition of lentivirus, Dynabeads were magnetically removed and media replaced bringing the cells to a final cell concentration of 0.5E6 per ml. Cells were cultured at 37° C. in a humidified incubator with 5% CO₂ and split as necessary during the expansion period. Cells were generally used or cryopreserved between 10 and 14 days post transduction with the “traditional manufacture” protocol.

For “shortened manufacture” CAR-T cell production and subsequent use in subsequent use in the in vitro and in vivo assays, purified human total T cells (CD4 and CD8) were collected from healthy donors and activated with Transact (1:17.5 v/v ratio, Miltenyi) in a shaker flask in complete medium then flask was placed into incubator (37° C. and 5% CO₂, passive humidity control) at 51 rpm. Shortened manufacture CAR-T cell complete medium was prepared using X-VIVO15 (Lonza)+40 u/mL IL-2 (Miltenyi)+0.24 u/mL IL-21 (Miltenyi)+1×ITSEA (InVitria). After overnight activation, lentivirus was added to T cells at a MOI=1.5 and additional complete medium was added to shaker at 2 hours post. The shaker speed was increased at this point to 69 rpm. Cells were monitored for viability and media was exchanged each day to keep cells at a concentration of 1.5e6 up to day 4. Cells were generally used or cryopreserved on day 4 post transduction with the “shortened manufacture” protocol.

To determine the level of CAR+ transduction, one of the following reagents or methods were used: Either an anti-Fab detection reagent labelled with AF647 (Jackson, cat 109-606-006), an anti-008LYG_D08 scFv reagent conjugated to Alexa-Fluor 647, or the GFP or mCherry protein co-expressed with CAR lentivirus were monitored by flow cytometry. To determine the level of dnTGFβRII surface expression, an anti-TGFβRII PE (BioLegend Cat #399703) was used. Cells were washed 3× in FACS buffer, and stained with the above mentioned reagents for 30 minutes at 4° C. kept in the dark. Cells were then washed an additional three times and resuspended in FACS buffer containing DAPI to gate on live/dead cells. Acquisition was performed with a FACSymphony instrument (BD) and data were analyzed using FlowJo software (Treestar, Ashland, OR).

For both “traditional” and “shortened manufacture” CAR-T cells, on the last day of expansion cells were cryopreserved using CryoStor® CS-10 Freeze Media (StemCell) and placed at a maximum of 100e6 cells per 1 ml CS-10, then vials were placed in a CoolCell Container (Corning) into a −80° C. freezer for 48 hours at which point vials were transferred to liquid nitrogen for extended storage.

### Quantitative Flow Cytometry for CLDN18.2 Expression

Quantitative surface expression of CLDN18.2 was carried out as described. For flow cytometry assessment of cell line CLDN18.2 expression, adherent cells were washed with PBS then removed from flask by TrypLE Express, resuspended in complete medium then counted using a Vi-Cell Blu Cell Viability Analyzer (Beckman Coulter, Indianapolis IN). Cells were plated in FACS buffer (1×PBS plus 2% FBS) at 2×10⁵ cells per well in duplicate in a round bottom 96 well plate and centrifuged at 1200 rpm, 4° C. for 4 minutes. Cells were kept at 4° C. (on ice) for the remainder of the assay. Cells were surface-stained with 10 μg/mL 008LY1_D04 or 008LYG_D08 directly conjugated to Alexa Fluor 647 and incubated in the dark for 30 minutes at 4° C. Cells were then washed three times and resuspended in FACS buffer containing DAPI to gate on live/dead cells. For quantitation purposes, Quantum™ Simply Cellular® beads (Bangs Laboratories, Inc., Fishers, IN) were also included in each assay and stained in the same manner as cancer cells. Data acquisition of cells and beads was performed with a FACSymphony instrument (BD) and data were analyzed using FlowJo software (Treestar, Ashland, OR). Mean fluorescence intensity (MFI) was translated to antibody binding capacity (ABC) values using the QuickCal analysis template provided by Bangs Laboratories, Inc.

### Modified Cell Line Generation Protocols

Initial flow cytometry with a CLDN18.2-specific reagent demonstrated a mixed/heterogeneous cell population of positive and negative cells in the PaTu 8988s “unsorted” cell line. Flow cytometry assisted cell sorting was used to generate the PaTu 8988s “high sort” cell line using a FACSAria Fusion Cell Sorter (BD), which produced a homogeneous and high expressing line.

For the CRISPR/Cas9 knock out of CLDN18.2, multi-guide RNA was purchased from Synthego (Redwood City, CA), Cas9 was purchased from Integrated DNA Technologies (IDT, Coralville, IA). Ribonucleoprotein (RNP) complexes were assembled as per Synthego suggested protocol (9:1 sgRNA to Cas9 ratio). Cells and pre-complexed RNP were mixed in an Eppendorf tube and transferred to a RUO OC-25×3 cassette (MaxCyte, Rockville, MD) and electroporated following optimization protocol 9 using an ExPERT GTx electroporation instrument (MaxCyte). Cells underwent three repeat rounds of this knock out protocol.

### Western Blot

CAR-T cells were sorted to generate a 100% pure CAR+ population using a FACSAria Fusion Cell Sorter (BD). These cells were then cocultured for various time periods with 1 ng/ml recombinant human TGFβ, at which point cells were placed on ice and lysed in RIPA buffer+1× proteases and phosphatases inhibitors for protein detection. Lysates were run through SDS-PAGE gel electrophoresis according to manufacturer's protocol with a Novex NuPage gel (4-12%), then transferred to a nitrocellulose membrane using an Invitrogen iBlot. Phospho SMAD-2/3 (Cell Signaling Technology (CST) Cat 8828S), total SMAD-2/3 (Cell Signaling Technology (CST) Cat 8685S) and β-actin (Sigma A3854) as a loading control were detected via HRP conjugated antibodies and ultra-sensitive enhanced chemiluminescent (ECL) substrate (Thermo Scientific) with the Image Quant biomolecular imaging system.

### Immunohistochemistry

Fresh tissues were collected and fixed in 10% Neutral Buffered Formalin for 24 hours, transferred in 70% ethanol prior to processing through a standard tissue processing methodology using a Tissue Tek Tissue Processor and subsequently embedded into paraffin blocks and stored at room temperature. Prior to the experiment, 5 μm tissue sections of each sample were baked at 60° C. for 1 hour.

Immunohistochemistry (IHC) was performed using an automated Leica Bond RX IHC staining platform (Leica, Milton Keynes, UK). Following antigen retrieval using Bond ER2 Solution for 30 minutes at 100° C., Peroxidase Block was incubated for 5 minutes. Primary CLDN18.2 antibody (Abcam, clone EPR19202) was incubated for 60 minutes at a concentration of either 0.5 or 1.0 μg/ml diluted in Dako diluent with background reducing components (Agilent cat S3022). IHC binding was demonstrated using the Bond Polymer Refine detection kit.

Protocol was similar for TGFβ, with the following modifications. Antigen retrieval using Bond ER2 Solution for 20 minutes at 100° C. followed by Peroxidase Block for 10 minutes and S-Block 1/1 (Ventana) for 15 minutes. Primary TGFβ1 antibody (Abcam ab215715) was incubated for 60 minutes at a concentration of 1.74 ug/ml (1:300) diluted in Dako diluent with background reducing components.

Primary phospho SMAD2 IHC was performed using a Ventana Discovery staining platform. Following deparaffinization, antigen retrieval was carried out in CC1 solution for 40 minutes at 98° C. and inhibitor was incubated for 12 minutes. Next, primary antibody (Cell Signaling Technology 138D4) was added at a concentration of 0.435 ug/ml (1:200) for 36 minutes at 36° C., secondary rabbit HRP for 16 minutes and DAB incubated for 8 minutes, hematoxylin for 12 minutes and bluing for 12 minutes.

Following cover slipping with DPX mounting media, slides were scanned, reviewed, and scored by a Pathologist assessing both the proportion of tumor cells expressing CLDN18.2, the intensity of staining, and cellular localisation of staining. All slides were digitally scanned using a Leica Aperio Scanscope AT2 pathology slide scanner (Leica, Milton Keynes, UK). CLDN18.2 IHC scores were generated using the following method: determine proportion of cells with any level of expression (0-4 scale), and then determine intensity of staining (1-3 scale). The total CLDN18.2 score (0-12) is determined by multiplying the proportion score by the intensity score. The same scoring system was used for TGFβ expression in the tumor cells. Scoring of the stromal component was carried out as follows, as the total amount of stromal cell presence varied among the tumor samples: total presence of stromal cells (0-3 scale) multiplied by overall intensity of staining (1-3 scale) to get to the stromal staining value (0-9).

In Vitro xCELLigence

Assessment of CAR-T activity in vitro was carried out using the Agilent xCELLigence Real-Time Cell Analysis system. On day 0, the instrument data collection schedule was set to measure impedance every 10 minutes for a 75 hour time period. Cancer cells were plated at optimal density as determined for each line that would result in a confluent monolayer (40,000-65,000 cells per well of a 96 well eSight plate) in final volume of 100 ul and the plate was allowed to sit for 30 minutes at room temperature before loading the plate into the eSight instrument. The following day, CAR-T cells were monitored for surface CAR+ by flow cytometry, then washed 3× of the CAR-T cell media and placed into the tumor cell complete media. CAR-T were added to the wells at a matched total CAR-T+ effector to target ratio and at a matched total T cell number, to account for differences in transduction efficiency among the clones to result in a final well volume of 200 ul. Tumor cell lysis is monitored in real time as the normalized cell index drops to 0 on the x-axis.

### ELISA

From the 96 well eSight plate used for the in vitro xCELLigence assay, at 24 hours post addition of CAR-T cells, 25 ul of the cell/CAR-T supernatant was carefully collected from the 200 ul in the well. Either a multi-spot V-Plex assay with capacity to detect pro-inflammatory cytokines IFN-γ, TNF-α or IL-2 (MSD, Rockville MD) in multiplex format or a single-plex IFN-γ ELISA system (R&D Systems, Minneapolis, MN) was used for downstream cytokine secretion assessment. For both ELISAs, assay was run according to manufacturer's protocol.

### Serial Antigen Restimulation Assay

To determine how many repeat rounds of serial antigen killing the CAR-T cells were able to carry out, a continuous in vitro co-culture assay was used to evaluate the ability of CAR-T cells for target killing and persistence capacity after multiple rounds of antigen challenge. On day 0, CAR-T cells and BxPC3 cells engineered to overexpress CLDN18.2 were plated at a 1:2 ET ratio and incubated for 3 days. CAR-T cells were then re-challenged with fresh BxPC3+CLDN18.2 cells every 3-4 days if CAR-T cells were still viable and lysing tumor cells, and flow cytometry was used to determine surface CAR+ expression and to maintain the same E:T ratio. After each round of re-challenge, CAR-T cells were counted to determine expansion, spun down at 16,000 rpms for 5 minutes, and then resuspended in fresh media. Culture supernatants were collected after 24 and 72 hours after each challenge. IFN-γ levels were measured by ELISA (R&D Systems). At the end of each antigen challenge, multi-parameter flow cytometry was carried out on the CAR-T to monitor cell memory phenotype, activation, and exhaustion markers. CAR-T cells were washed in FACS buffer, then incubated with an antibody cocktail for markers including but not limited to CD8, CD4, CD45, CD62L, CD45RO, CD70, CD27, CD223, PD-1, LAG3 and TIM3 (BioLegend, BD BioSciences, and R&D Systems) and incubated for 30 minutes on ice. CAR positivity was determined by using an anti-008LYG_D08 scFv reagent conjugated to Alexa-Fluor 647 antibody and/or anti-TGFβRII conjugated to PE antibody. A live/dead stain (ThermoFisher) was used to exclude dead cells. In addition, BxPC3+CLDN18.2 cell killing was determined by using CellTiter-Glo® reagent (Promega).

### In Vivo Animal Studies:

All animal experiments were conducted in a facility accredited by the Association for Assessment of Laboratory Animal Care (AALAC) under Institutional Animal Care and Use Committee (IACUC) guidelines and appropriate animal research approval. Studies used 6 to 8-week-old female NOD. Cg-PrkdcscidIl2rgtm1Wjl/SzJ (Jackson Laboratories, NSG) mice or 6 to 8-week-old female NOD. Cg-Prkdcscid H2-Ab 1em1Mvw H2-K1tm1BpeH2-D1tm1Bpe Il2rgtm1Wjl/SzJ (Jackson, NSG MHC I/II KO) mice as noted.

Cultrex Basement Membrane Extract (BME), Type 3 is an extracellular matrix hydrogel that is qualified specifically for use in in vivo xenograft and tumorgraft models. It is used at a 1:1 ration (PBS:BME). Each mouse is injected s.c. with 200 ul total volume. 10 million PaTu 8988S “high sort” cells were injected into the right flank in a total volume of 200 ul (PBS:BME, 1:1). For NCI-N87 engineered to overexpress CLDN18.2, 5 million cells were injected into right flank in total volume of 200 ul (PBS:BME, 1:1).

For PDX tumor fragment implantation: Tumor tissue was placed in a sterile petri dish with basal RPMI media and cut into small pieces, approximately 3 mm×3 mm. Freshly harvested tissue or thawed fragments from frozen stocks may be used. Fragments may be implanted using trocar as follows: The 3 mm×3 mm fragment is placed into a 11 gauge trocar. The trocar is then used to deliver the tumor fragment subcutaneously into the right flank of the mouse.

For all studies, tumor volumes and body weight was measured twice weekly for the duration of the study.

### Syngeneic Murine Models of Melanoma and Colon Carcinoma

FIGS. 15A-15F show data from syngeneic murine models of melanoma (B16-F10) and colon carcinoma (CT-26) engineered to express murine CLDN18.2. This model was used to show efficacy of CAR-T cells bearing the 008LYG_DO8 scFv, but in this case engineered with a murine CD28 costimulatory domain and a murine CD3z signaling domain. The CAR-T cells were further armored with a murine dominant negative TGFbRII. Because the 008LYG_DO8 scFv is murine cross reactive these models can be used to examine efficacy of 008LYG_DO8 CAR-T engineered with a murine CD28 and murine CD3z, as well as the murine dominant negative TGFbRII, in immune competent mice following lymphodepletion.

In vivo efficacy studies of 008LYG_DO8 mCD28z m-dnTGFbRII CAR-T were carried out in 6 to 8-week-old female BALB/cJ mice (Jackson Laboratories) mice. On day −7, 5e5 CT-26 WT or CT-26+mCLDN18.2 cells were implanted subcutaneously in the upper right flank. On day −1, mice were subjected to whole body irradiation at 3 Gy as a means of lymphodepletion, and on day 0, when xenografts reached an average size of 150 to 215 mm³ (CT-26+mCLDN18.2 tumors were slightly larger than wt on randomization), mice were dosed intravenously with a single infusion of 9e6 008LYG_D08 mCD28z m-dnTGFbRII CAR-T cells, or 9e6 untransduced donor matched murine T cells. This single murine CAR-T infusion led to tumor growth inhibition specifically in CLDN18.2 expressing xenografts (FIG. 15F), with no significant body weight loss (data not shown).

### Discussion:

While promising clinical data has been demonstrated using CAR-Ts targeting CD19 and BCMA in hematologic cancers (J Hematol Oncol Pharm. 2022; 12(1):30-42, Leukemia volume 36, pages1481-1484, 2022), translation of this success to the solid tumor setting has not been straightforward. It is well known that there are multiple barriers in which engineered CAR-T cells will face within the solid tumor microenvironment, including but not limited to heterogeneity of tumor target antigen expression, impaired trafficking and infiltration of CAR-T cells into the tumor and the highly immunosuppressive and hostile tumor microenvironment which CAR-T cells will face on arrival. Therefore, careful consideration of CAR-T design, ideal infusion CAR-T phenotype and optimized manufacture are needed to achieve clinical responses in solid tumors with this therapeutic approach.

CLDN18.2 is an antigen being explored clinically via multiple modalities including ADCC and CDC inducing monoclonal antibodies, antibody drug conjugates, T cell engagers and CAR-Ts in gastric, pancreatic and gastroesophageal junction cancers. It is known that CLDN18.2 is also expressed on the differentiated cells of the normal gastric mucosa, so identification of an optimal safety window will be of utmost importance. Interim analysis of clinical data following administration of a CLDN18.2 targeting CAR-T CT041 showed antitumor efficacy and a tolerable safety profile in gastric cancer patients (Nature Medicine volume 28, pages1189-1198 (2022)). The durability of tumor response in the hematologic setting has been linked to persistence of CAR-T cells in the patient. Median persistence of CT041 following the first infusion was 28 days in the peripheral blood (Nature Medicine volume 28, pages1189-1198 (2022), which can indicate room for improvement of the CAR-T product. In this disclosure, identification of a lead CAR-T construct which maintained murine cross reactivity enabled early assessment of safety in addition to efficacy readouts in a relevant rodent model and optimization of CAR-T design for maximum potential clinical benefit. Armoring, e.g with a dominant negative TGFβRII, may improve persistence of the CLDN18.2 CAR-Ts of this disclosure in the clinical setting. This is supported by the data shown in FIG. 10 of long lasting in vivo efficacy in the Panc06 PDX model.

The critical components of a functional CAR are the antigen binding domain and the one or more intracellular domains (e.g., costimulatory domains and/or signaling domains). Herein, data has been disclosed that strongly supports that CARs based on the 008LYG_D08 scFv exhibit potent cytotoxic effects on CLDN18.2 expressing cell lines in vitro (FIG. 5C, 5E) and in vivo (FIG. 7D). Moreover, the unique characteristics inherent to 008LYG_D08 scFv, including its low CLDN18.2 affinity, membrane-proximal and conformational epitope, and internalization phenotype (FIG. 4), are responsible for the preferred behavior in CAR-T cell therapies. Additionally, data disclosed within demonstrate that the 008LYG_D08 CD28z CAR-T is capable of inducing tumor cell lysis in cancer cells presenting with high, moderate and low CLDN18.2 surface receptor density levels, indicating that the disclosed 008LYG_D08 CD28z CAR-T may be effective in a heterogeneous tumor with varied levels of receptor density present (Table 3).

Specificity of CAR-T cell killing in cells which express CLDN18.2 was demonstrated by lack of activity in cells which had the target antigen knocked out by CRISPR/Cas9 (FIG. 6B).

The addition of a dominant negative TGFβRII will also impart benefit to the 008LYG_D08 CD28z CAR-T cells by enabling the cells to overcome excessive and suppressive TGFβ that is shown to be present in the tumor microenvironment of gastric, pancreatic and esophageal cancers (FIG. 8C-E). This document discloses data which demonstrates that inclusion of this armoring strategy in the lentiviral construct can block downstream phosphorylation of SMAD2/3 (FIG. 8H), leading to generation of CAR-T cells which can proliferate and persist through multiple rounds of antigen rechallenge and continue to lyse tumor cells beyond an unarmored counterpart (FIG. 8I).

The added benefit of the armoring was further exampled in animal models bearing xenografts of a pancreatic cell line that endogenously (FIG. 9A-D) or a gastric cell line engineered to overexpress (FIG. 9E-H) CLDN18.2 as well as in a pancreatic cancer patient derived xenograft model (FIG. 9I-L), all with varied levels of both CLDN18.2 and TGFβ1 expression. In all cases, the 008LYG_D08 CD28z dnTGFβRII armored CAR-T was able to produce efficacious tumor regressions which were long lasting and not associated with any body weight loss, indicative of a promising CAR-T product.

Optimized and “shortened” manufacturing (SMART) produced a CAR-T cell product which was able to persist longer, maintain a less differentiated T cell phenotype, and maintain tumor cell lysis for additional rounds of restimulation compared to a “traditional” manufactured product (FIG. 10A). The “shortened” manufacturing translated to a CAR-T which presented with a less differentiated phenotype as monitored by cell surface CD62L/CR45RO expression. (FIG. 10B) which altogether may clinically translate to a CAR-T product with potential for efficacy at reduced infusion dose and persistence of CAR-T function. Long lasting tumor regressions were observed at low CAR-T infusion dose in a representative patient derived xenograft model of pancreatic cancer (FIG. 10E) in which no body weight loss was observed (FIG. 10F). Sustained tumor regression was also observed in xenograph models of pancreatic cancer at low CAR-T infusion dose obtained from a second T-cell donor using the optimized and “shortened” manufacturing process (FIG. 13).

Long lasting tumor regression was also observed at low CAR-T infusion dose in representative patient derived xenograft models of gastric (FIG. 11) and esophageal (FIG. 12) cancer.

Syngeneic murine models of melanoma and colon carcinoma were used to demonstrate the efficacy of the disclosed embodiments in immune competent mice. In particular, CAR-T cells bearing the 008LYG_DO8 scFv coupled to murine CD28 costimulatory and murine CD3z signaling domains, and armored using murine dominant negative TGF-beta, inhibited tumor growth that was specific to CLDN18.2 expressing xenografts in immune competent mice (FIG. 15F).

Altogether, the components disclosed within demonstrate a CAR-T product comprised of a unique and potent scFv, dominant negative TGFβ armoring and produced utilizing a shortened manufacture process to generate a favorable CAR-T infusion cell phenotype lend to a well differentiated final CAR-T product with demonstrated efficacy and therapeutic potential.

TABLE 4
Sequences.

| SEQ | | |
|---|---|---|
| ID | Description | Sequences |
| 1 | 008LYG_D08 CDR H1 | SYAIS |
| 2 | 008LYG_D08 CDR H2 | GIIPIFGTANYAQKFQG |
| 3 | 008LYG_D08 CDR H3 | GYQGMGVFDP |
| 4 | 008LYG_D08 CDR L1 | SGSSSNIGSNTVN |
| 5 | 008LYG_D08 CDR L2 | SNNQRPS |
| 6 | 008LYG_D08 CDR L3 | AAWDALDWVVV |
| 7 | 008LYG_D08 VH | QVQLVQSGAEVKKPGSSVKVSCKASGGTFSSYAISWVRQAPGQGLEWMGGIIPIFGTANY |
| | | AQKFQGRVTITADESTSTAYMELSSLRSEDTAVYYCARGYQGMGVFDPWGQGTLVTVSS |
| 8 | 008LYG_D08 VL | QSVLTQPPSASGTPGQRVTISCSGSSSNIGSNTVNWYQQLPGTAPKLLIYSNNQRPSGVP |
| | | DRFSGSKSGTSASLAISGLQSEDEADYYCAAWDALDWVVVFGGGTKLTVL |
| 9 | 008LYG_D08 scFv | QVQLVQSGAEVKKPGSSVKVSCKASGGTFSSYAISWVRQAPGQGLEWMGGIIPIFGTANY |
| | | AQKFQGRVTITADESTSTAYMELSSLRSEDTAVYYCARGYQGMGVFDPWGQGTLVTVSSG |
| | | GGGSGGGGSGGGGSAQSVLTQPPSASGTPGQRVTISCSGSSSNIGSNTVNWYQQLPGTAP |
| | | KLLIYSNNQRPSGVPDRFSGSKSGTSASLAISGLQSEDEADYYCAAWDALDWVVVFGGGT |
| | | KLTVL |
| 10 | 008LYG_D08 Bz CAR- | MPLLLLLPLLWAGALAQVQLVQSGAEVKKPGSSVKVSCKASGGTFSSYAISWVRQAPGQG |
| | T2A-GFP | LEWMGGIIPIFGTANYAQKFQGRVTITADESTSTAYMELSSLRSEDTAVYYCARGYQGMG |
| | | VFDPWGQGTLVTVSSGGGGSGGGGSGGGGSAQSVLTQPPSASGTPGQRVTISCSGSSSNI |
| | | GSNTVNWYQQLPGTAPKLLIYSNNQRPSGVPDRFSGSKSGTSASLAISGLQSEDEADYYC |
| | | AAWDALDWVVVFGGGTKLTVLESKYGPPCPPCPFWVLVVVGGVLACYSLLVTVAFIIFWV |
| | | KRGRKKLLYIFKQPFMRPVQTTQEEDGCSCRFPEEEEGGCELRVKFSRSADAPAYQQGQN |
| | | QLYNELNLGRREEYDVLDKRRGRDPEMGGKPRRKNPQEGLYNELQKDKMAEAYSEIGMKG |
| | | ERRRGKGHDGLYQGLSTATKDTYDALHMQALPPREFEGSAAAEGRGSLLTCGDVEENPGP |
| | | SGMESDESGLPAMEIECRITGTLNGVEFELVGGGEGTPKQGRMTNKMKSTKGALTFSPYL |
| | | LSHVMGYGFYHFGTYPSGYENPFLHAINNGGYTNTRIEKYEDGGVLHVSFSYRYEAGRVI |
| | | GDFKVVGTGFPEDSVIFTDKIIRSNATVEHLHPMGDNVLVGSFARTFSLRDGGYYSFVVD |
| | | SHMHFKSAIHPSILQNGGPMFAFRRVEELHSNTELGIVEYQHAFKTPIAFARSRAQSSNS |
| | | AVDGTAGPGSTGSR |
| 11 | 008LY1_D04 CDR H1 | SYAIS |
| 12 | 008LY1_D04 CDR H2 | GIIPIFGTANYAQKFQG |
| 13 | 008LY1_D04 CDR H3 | GYVQYGYFDY |
| 14 | 008LY1_D04 CDR L1 | SGSSSNIGSNTVN |
| 15 | 008LY1_D04 CDR L2 | SNNQRPS |
| 16 | 008LY1_D04 CDR L3 | AAWDTVDYVV |
| 17 | 008LY1_D04 VH | QVQLVQSGAEVKKPGSSVKVSCKASGGTFSSYAISWVRQAPGQGLEWMGGIIPIFGTANY |
| | | AQKFQGRVTITADESTSTAYMELSSLRSEDTAVYYCARGYVQYGYFDYWGQGTLVTVSS |
| 18 | 008LY1_D04 VL | QSVLTQPPSASGTPGQRVTISCSGSSSNIGSNTVNWYQQLPGTAPKLLIYSNNQRPSGVP |
| | | DRFSGSKSGTSASLAISGLQSEDEADYYCAAWDTVDYVVFGGGTKLTVL |
| 19 | 008LY1_D04 scFv | QVQLVQSGAEVKKPGSSVKVSCKASGGTFSSYAISWVRQAPGQGLEWMGGIIPIFGTANY |
| | | AQKFQGRVTITADESTSTAYMELSSLRSEDTAVYYCARGYVQYGYFDYWGQGTLVTVSSG |
| | | GGGSGGGGSGGGGSAQSVLTQPPSASGTPGQRVTISCSGSSSNIGSNTVNWYQQLPGTAP |
| | | KLLIYSNNQRPSGVPDRFSGSKSGTSASLAISGLQSEDEADYYCAAWDTVDYVVFGGGTK |
| | | LTVL |
| 20 | 008LY1_D04 Bz CAR- | MPLLLLLPLLWAGALAQVQLVQSGAEVKKPGSSVKVSCKASGGTFSSYAISWVRQAPGQG |
| | T2A-GFP | LEWMGGIIPIFGTANYAQKFQGRVTITADESTSTAYMELSSLRSEDTAVYYCARGYVQYG |
| | | YFDYWGQGTLVTVSSGGGGSGGGGSGGGGSAQSVLTQPPSASGTPGQRVTISCSGSSSNI |
| | | GSNTVNWYQQLPGTAPKLLIYSNNQRPSGVPDRFSGSKSGTSASLAISGLQSEDEADYYC |
| | | AAWDTVDYVVFGGGTKLTVLESKYGPPCPPCPFWVLVVVGGVLACYSLLVTVAFIIFWVK |
| | | RGRKKLLYIFKQPFMRPVQTTQEEDGCSCRFPEEEEGGCELRVKFSRSADAPAYQQGQNQ |
| | | LYNELNLGRREEYDVLDKRRGRDPEMGGKPRRKNPQEGLYNELQKDKMAEAYSEIGMKGE |
| | | RRRGKGHDGLYQGLSTATKDTYDALHMQALPPREFEGSAAAEGRGSLLTCGDVEENPGPS |
| | | GMESDESGLPAMEIECRITGTLNGVEFELVGGGEGTPKQGRMTNKMKSTKGALTFSPYLL |
| | | SHVMGYGFYHFGTYPSGYENPFLHAINNGGYTNTRIEKYEDGGVLHVSFSYRYEAGRVIG |
| | | DFKVVGTGFPEDSVIFTDKIIRSNATVEHLHPMGDNVLVGSFARTFSLRDGGYYSFVVDS |
| | | HMHFKSAIHPSILQNGGPMFAFRRVEELHSNTELGIVEYQHAFKTPIAFARSRAQSSNSA |
| | | VDGTAGPGSTGSR |
| 21 | ZP1I16_D05 CDR H1 | SYAIS |
| 22 | ZP1I16_D05 CDR H2 | GIIPIFGTANYAQKFQG |
| 23 | ZP1I16_D05 CDR H3 | GFIRYGVFDY |
| 24 | ZP1I16_D05 CDR L1 | SGSSSNIGSNTVN |
| 25 | ZP1I16_D05 CDR L2 | SNNQRPS |
| 26 | ZP1I16_D05 CDR L3 | AAWDDQRLHVV |
| 27 | ZP1I16_D05 VH | QVQLVQSGAEVKKPGSSVKVSCKASGGTFSSYAISWVRQAPGQGLEWMGGIIPIFGTANY |
| | | AQKFQGRVTITADESTSTAYMELSSLRSEDTAVYYCARGFIRYGVFDYWGQGTLVTVSS |
| 28 | ZP1I16_D05 VL | QSVLTQPPSASGTPGQRVTISCSGSSSNIGSNTVNWYQQLPGTAPKLLIYSNNQRPSGVP |
| | | DRFSGSKSGTSASLAISGLQSEDEADYYCAAWDDQRLHVVFGGGTKLTVL |
| 29 | ZP1I16_D05 scFv | QVQLVQSGAEVKKPGSSVKVSCKASGGTFSSYAISWVRQAPGQGLEWMGGIIPIFGTANY |
| | | AQKFQGRVTITADESTSTAYMELSSLRSEDTAVYYCARGFIRYGVFDYWGQGTLVTVSSG |
| | | GGGSGGGGSGGGGSAQSVLTQPPSASGTPGQRVTISCSGSSSNIGSNTVNWYQQLPGTAP |
| | | KLLIYSNNQRPSGVPDRFSGSKSGTSASLAISGLQSEDEADYYCAAWDDQRLHVVFGGGT |
| | | KLTVL |
| 30 | ZP1I16_D05 Bz CAR- | MPLLLLLPLLWAGALAQVQLVQSGAEVKKPGSSVKVSCKASGGTFSSYAISWVRQAPGQG |
| | T2A-GFP | LEWMGGIIPIFGTANYAQKFQGRVTITADESTSTAYMELSSLRSEDTAVYYCARGFIRYG |
| | | VFDYWGQGTLVTVSSGGGGSGGGGSGGGGSAQSVLTQPPSASGTPGQRVTISCSGSSSNI |
| | | GSNTVNWYQQLPGTAPKLLIYSNNQRPSGVPDRFSGSKSGTSASLAISGLQSEDEADYYC |
| | | AAWDDQRLHVVFGGGTKLTVLESKYGPPCPPCPFWVLVVVGGVLACYSLLVTVAFIIFWV |
| | | KRGRKKLLYIFKQPFMRPVQTTQEEDGCSCRFPEEEEGGCELRVKFSRSADAPAYQQGQN |
| | | QLYNELNLGRREEYDVLDKRRGRDPEMGGKPRRKNPQEGLYNELQKDKMAEAYSEIGMKG |
| | | ERRRGKGHDGLYQGLSTATKDTYDALHMQALPPREFEGSAAAEGRGSLLTCGDVEENPGP |
| | | SGMESDESGLPAMEIECRITGTLNGVEFELVGGGEGTPKQGRMTNKMKSTKGALTFSPYL |
| | | LSHVMGYGFYHFGTYPSGYENPFLHAINNGGYTNTRIEKYEDGGVLHVSFSYRYEAGRVI |
| | | GDFKVVGTGFPEDSVIFTDKIIRSNATVEHLHPMGDNVLVGSFARTFSLRDGGYYSFVVD |
| | | SHMHFKSAIHPSILQNGGPMFAFRRVEELHSNTELGIVEYQHAFKTPIAFARSRAQSSNS |
| | | AVDGTAGPGSTGSR |
| 31 | 008M0G_G03 CDR H1 | SYAIS |
| 32 | 008M0G_G03 CDR H2 | GIIPIFGTANYAQKFQG |
| 33 | 008M0G_G03 CDR H3 | GYPRYGVFDY |
| 34 | 008M0G_G03 CDR L1 | SGSSSNIGSNTVN |
| 35 | 008M0G_G03 CDR L2 | SNNQRPS |
| 36 | 008M0G_G03 CDR L3 | AAWDRYRFHVV |
| 37 | 008M0G_G03 VH | QVQLVQSGAEVKKPGSSVKVSCKASGGTFSSYAISWVRQAPGQGLEWMGGIIPIFGTANY |
| | | AQKFQGRVTITADESTSTAYMELSSLRSEDTAVYYCARGYPRYGVFDYWGQGTLVTVSS |
| 38 | 008M0G_G03 VL | QSVLTQPPSASGTPGQRVTISCSGSSSNIGSNTVNWYQQLPGTAPKLLIYSNNQRPSGVP |
| | | DRFSGSKSGTSASLAISGLQSEDEADYYCAAWDRYRFHVVFGGGTKLTVL |
| 39 | 008M0G_G03 scFv | QVQLVQSGAEVKKPGSSVKVSCKASGGTFSSYAISWVRQAPGQGLEWMGGIIPIFGTANY |
| | | AQKFQGRVTITADESTSTAYMELSSLRSEDTAVYYCARGYPRYGVFDYWGQGTLVTVSSG |
| | | GGGSGGGGSGGGGSAQSVLTQPPSASGTPGQRVTISCSGSSSNIGSNTVNWYQQLPGTAP |
| | | KLLIYSNNQRPSGVPDRFSGSKSGTSASLAISGLQSEDEADYYCAAWDRYRFHVVFGGGT |
| | | KLTVL |
| 40 | 008M0G_G03 Bz CAR- | MPLLLLLPLLWAGALAQVQLVQSGAEVKKPGSSVKVSCKASGGTFSSYAISWVRQAPGQG |
| | T2A-GFP | LEWMGGIIPIFGTANYAQKFQGRVTITADESTSTAYMELSSLRSEDTAVYYCARGYPRYG |
| | | VFDYWGQGTLVTVSSGGGGSGGGGSGGGGSAQSVLTQPPSASGTPGQRVTISCSGSSSNI |
| | | GSNTVNWYQQLPGTAPKLLIYSNNQRPSGVPDRFSGSKSGTSASLAISGLQSEDEADYYC |
| | | AAWDRYRFHVVFGGGTKLTVLESKYGPPCPPCPFWVLVVVGGVLACYSLLVTVAFIIFWV |
| | | KRGRKKLLYIFKQPFMRPVQTTQEEDGCSCRFPEEEEGGCELRVKFSRSADAPAYQQGQN |
| | | QLYNELNLGRREEYDVLDKRRGRDPEMGGKPRRKNPQEGLYNELQKDKMAEAYSEIGMKG |
| | | ERRRGKGHDGLYQGLSTATKDTYDALHMQALPPREFEGSAAAEGRGSLLTCGDVEENPGP |
| | | SGMESDESGLPAMEIECRITGTLNGVEFELVGGGEGTPKQGRMTNKMKSTKGALTFSPYL |
| | | LSHVMGYGFYHFGTYPSGYENPFLHAINNGGYTNTRIEKYEDGGVLHVSFSYRYEAGRVI |
| | | GDFKVVGTGFPEDSVIFTDKIIRSNATVEHLHPMGDNVLVGSFARTFSLRDGGYYSFVVD |
| | | SHMHFKSAIHPSILQNGGPMFAFRRVEELHSNTELGIVEYQHAFKTPIAFARSRAQSSNS |
| | | AVDGTAGPGSTGSR |
| 41 | ZP1I18_B08 CDR H1 | SYAIS |
| 42 | ZP1I18_B08 CDR H2 | GIIPIFGTANYAQKFQG |
| 43 | ZP1I18_B08 CDR H3 | GFIRYGVFDY |
| 44 | ZP1118_B08 CDR L1 | SGSSSNIGSNTVN |
| 45 | ZP1I18_B08 CDR L2 | SNNQRPS |
| 46 | ZP1I18_B08 CDR L3 | AAWDDRFHHVV |
| 47 | ZP1I18_B08 VH | QVQLVQSGAEVKKPGSSVKVSCKASGGTFSSYAISWVRQAPGQGLEWMGGIIPIFGTANY |
| | | AQKFQGRVTITADESTSTAYMELSSLRSEDTAVYYCARGFIRYGVFDYWGQGTLVTVSS |
| 48 | ZP1I18_B08 VL | QSVLTQPPSASGTPGQRVTISCSGSSSNIGSNTVNWYQQLPGTAPKLLIYSNNQRPSGVP |
| | | DRFSGSKSGTSASLAISGLQSEDEADYYCAAWDDRFHHVVFGGGTKLTVL |
| 49 | ZP1I18_B08 scFv | QVQLVQSGAEVKKPGSSVKVSCKASGGTFSSYAISWVRQAPGQGLEWMGGIIPIFGTANY |
| | | AQKFQGRVTITADESTSTAYMELSSLRSEDTAVYYCARGFIRYGVFDYWGQGTLVTVSSG |
| | | GGGSGGGGSGGGGSAQSVLTQPPSASGTPGQRVTISCSGSSSNIGSNTVNWYQQLPGTAP |
| | | KLLIYSNNQRPSGVPDRFSGSKSGTSASLAISGLQSEDEADYYCAAWDDRFHHVVFGGGT |
| | | KLTVL |
| 50 | ZP1I18_B08 Bz CAR- | MPLLLLLPLLWAGALAQVQLVQSGAEVKKPGSSVKVSCKASGGTFSSYAISWVRQAPGQG |
| | T2A-GFP | LEWMGGIIPIFGTANYAQKFQGRVTITADESTSTAYMELSSLRSEDTAVYYCARGFIRYG |
| | | VFDYWGQGTLVTVSSGGGGSGGGGSGGGGSAQSVLTQPPSASGTPGQRVTISCSGSSSNI |
| | | GSNTVNWYQQLPGTAPKLLIYSNNQRPSGVPDRFSGSKSGTSASLAISGLQSEDEADYYC |
| | | AAWDDRFHHVVFGGGTKLTVLESKYGPPCPPCPFWVLVVVGGVLACYSLLVTVAFIIFWV |
| | | KRGRKKLLYIFKQPFMRPVQTTQEEDGCSCRFPEEEEGGCELRVKFSRSADAPAYQQGQN |
| | | QLYNELNLGRREEYDVLDKRRGRDPEMGGKPRRKNPQEGLYNELQKDKMAEAYSEIGMKG |
| | | ERRRGKGHDGLYQGLSTATKDTYDALHMQALPPREFEGSAAAEGRGSLLTCGDVEENPGP |
| | | SGMESDESGLPAMEIECRITGTLNGVEFELVGGGEGTPKQGRMTNKMKSTKGALTFSPYL |
| | | LSHVMGYGFYHFGTYPSGYENPFLHAINNGGYTNTRIEKYEDGGVLHVSFSYRYEAGRVI |
| | | GDFKVVGTGFPEDSVIFTDKIIRSNATVEHLHPMGDNVLVGSFARTFSLRDGGYYSFVVD |
| | | SHMHFKSAIHPSILQNGGPMFAFRRVEELHSNTELGIVEYQHAFKTPIAFARSRAQSSNS |
| | | AVDGTAGPGSTGSR |
| 51 | 008LYG_D08 CD28z | ATGCCTTTGTTGCTGCTGCTGCCTCTTCTTTGGGCTGGCGCGCTAGCCCAGGTTCAGCTT |
| | CAR Unarmored | GTTCAATCTGGCGCCGAAGTGAAGAAACCCGGCAGCTCTGTGAAGGTGTCCTGCAAAGCT |
| | nucleic acid | AGCGGCGGCACCTTTAGCAGCTACGCCATCTCTTGGGTCCGACAGGCTCCTGGACAAGGC |
| | sequence | CTGGAATGGATGGGCGGCATCATCCCTATCTTCGGCACCGCCAATTACGCCCAGAAATTC |
| | | CAGGGCAGAGTGACCATCACCGCCGACGAGTCTACAAGCACCGCCTACATGGAACTGAGC |
| | | AGCCTGAGAAGCGAGGACACCGCCGTGTACTATTGCGCCAGAGGCTATCAAGGCATGGGC |
| | | GTGTTCGATCCTTGGGGCCAGGGAACACTGGTCACAGTTTCTAGCGGAGGCGGAGGATCT |
| | | GGTGGCGGAGGAAGTGGCGGAGGCGGTTCTGCTCAATCTGTGCTGACACAGCCTCCTAGC |
| | | GCCTCTGGAACACCTGGCCAGAGAGTGACAATCAGCTGTAGCGGCAGCAGCTCCAACATC |
| | | GGCAGCAACACCGTGAACTGGTATCAGCAGCTGCCTGGCACAGCCCCTAAACTGCTGATC |
| | | TACAGCAACAACCAGCGGCCTAGCGGCGTGCCCGATAGATTTTCTGGCAGCAAGAGCGGC |
| | | ACAAGCGCCAGCCTGGCTATTTCTGGACTGCAGAGCGAGGACGAGGCCGACTATTATTGT |
| | | GCCGCCTGGGATGCCCTGGACTGGGTCGTCGTTTTTGGCGGCGGAACAAAGCTGACCGTG |
| | | CTCGAGAGCAAATATGGACCACCATGCCCTCCATGTCCTTTCTGGGTGCTCGTGGTTGTT |
| | | GGCGGAGTGCTGGCCTGTTACAGCCTGCTGGTTACCGTGGCCTTCATCATCTTTTGGGTC |
| | | CGAAGCAAGCGGAGCAGACTGCTGCACAGCGACTACATGAACATGACCCCTAGACGGCCC |
| | | GGACCTACCAGAAAGCACTACCAGCCTTACGCTCCTCCTAGAGACTTCGCCGCCTACAGA |
| | | AGCCGCGTGAAGTTTTCTCGAAGTGCCGATGCTCCTGCATATCAGCAGGGACAGAACCAG |
| | | CTGTACAACGAGCTGAATCTGGGCCGGAGAGAGGAATACGACGTGCTGGATAAGAGGCGC |
| | | GGCAGAGACCCAGAAATGGGCGGGAAGCCACGACGGAAAAACCCCCAGGAGGGGCTGTAT |
| | | AATGAACTGCAGAAGGACAAAATGGCCGAGGCTTACAGCGAAATCGGGATGAAGGGAGAG |
| | | AGAAGGCGCGGAAAAGGCCACGATGGACTGTATCAGGGCCTGAGCACTGCCACCAAGGAC |
| | | ACCTACGATGCTCTGCACATGCAGGCACTGCCACCCAGGTAA |
| 52 | 008LYG_D08 CD28z | MPLLLLLPLLWAGALAQVQLVQSGAEVKKPGSSVKVSCKASGGTFSSYAISWVRQAPGQG |
| | CAR Unarmored | LEWMGGIIPIFGTANYAQKFQGRVTITADESTSTAYMELSSLRSEDTAVYYCARGYQGMG |
| | amino acid | VFDPWGQGTLVTVSSGGGGSGGGGSGGGGSAQSVLTQPPSASGTPGQRVTISCSGSSSNI |
| | sequence | GSNTVNWYQQLPGTAPKLLIYSNNQRPSGVPDRFSGSKSGTSASLAISGLQSEDEADYYC |
| | | AAWDALDWVVVFGGGTKLTVLESKYGPPCPPCPFWVLVVVGGVLACYSLLVTVAFIIFWV |
| | | RSKRSRLLHSDYMNMTPRRPGPTRKHYQPYAPPRDFAAYRSRVKFSRSADAPAYQQGQNQ |
| | | LYNELNLGRREEYDVLDKRRGRDPEMGGKPRRKNPQEGLYNELQKDKMAEAYSEIGMKGE |
| | | RRRGKGHDGLYQGLSTATKDTYDALHMQALPPR |
| 53 | Dominant-negative | ATGGGTCGGGGGCTGCTCAGGGGCCTGTGGCCGCTGCACATCGTCCTGTGGACGCGTATC |
| | TGFβ receptor | GCCAGCACGATCCCACCGCACGTTCAGAAGTCGGTTAATAACGACATGATAGTCACTGAC |
| | type 2 nucleic | AACAACGGTGCAGTCAAGTTTCCACAACTGTGTAAATTTTGTGATGTGAGATTTTCCACC |
| | acid sequence | TGTGACAACCAGAAATCCTGCATGAGCAACTGCAGCATCACCTCCATCTGTGAGAAGCCA |
| | | CAGGAAGTCTGTGTGGCTGTATGGAGAAAGAATGACGAGAACATAACACTAGAGACAGTT |
| | | TGCCATGACCCCAAGCTCCCCTACCATGACTTTATTCTGGAAGATGCTGCTTCTCCAAAG |
| | | TGCATTATGAAGGAAAAGAAAAAGCCTGGTGAGACTTTCTTCATGTGTTCCTGTAGCTCT |
| | | GATGAGTGCAATGACAACATCATCTTCTCAGAAGAATATAACACCAGCAATCCTGACTTG |
| | | TTGCTAGTCATATTTCAAGTGACAGGCATCAGCCTCCTGCCACCACTGGGAGTTGCCATA |
| | | TCTGTCATCATCATCTTCTACTGCTACCGCGTTAACCGGCAGTAA |
| 54 | Dominant-negative | MGRGLLRGLWPLHIVLWTRIASTIPPHVQKSVNNDMIVTDNNGAVKFPQLCKFCDVRFST |
| | TGFβ receptor | CDNQKSCMSNCSITSICEKPQEVCVAVWRKNDENITLETVCHDPKLPYHDFILEDAASPK |
| | type 2 amino | CIMKEKKKPGETFFMCSCSSDECNDNIIFSEEYNTSNPDLLLVIFQVTGISLLPPLGVAI |
| | acid sequence | SVIIIFYCYRVNRQ |
| 55 | 008LYG_D08 CD28Z | ATGCCTTTGTTGCTGCTGCTGCCTCTTCTTTGGGCTGGCGCGCTAGCCCAGGTTCAGCTT |
| | CAR-T2A-Dominant- | GTTCAATCTGGCGCCGAAGTGAAGAAACCCGGCAGCTCTGTGAAGGTGTCCTGCAAAGCT |
| | negative TGFβ | AGCGGCGGCACCTTTAGCAGCTACGCCATCTCTTGGGTCCGACAGGCTCCTGGACAAGGC |
| | receptor type 2 | CTGGAATGGATGGGCGGCATCATCCCTATCTTCGGCACCGCCAATTACGCCCAGAAATTC |
| | nucleic acid | CAGGGCAGAGTGACCATCACCGCCGACGAGTCTACAAGCACCGCCTACATGGAACTGAGC |
| | sequence | AGCCTGAGAAGCGAGGACACCGCCGTGTACTATTGCGCCAGAGGCTATCAAGGCATGGGC |
| | | GTGTTCGATCCTTGGGGCCAGGGAACACTGGTCACAGTTTCTAGCGGAGGCGGAGGATCT |
| | | GGTGGCGGAGGAAGTGGCGGAGGCGGTTCTGCTCAATCTGTGCTGACACAGCCTCCTAGC |
| | | GCCTCTGGAACACCTGGCCAGAGAGTGACAATCAGCTGTAGCGGCAGCAGCTCCAACATC |
| | | GGCAGCAACACCGTGAACTGGTATCAGCAGCTGCCTGGCACAGCCCCTAAACTGCTGATC |
| | | TACAGCAACAACCAGCGGCCTAGCGGCGTGCCCGATAGATTTTCTGGCAGCAAGAGCGGC |
| | | ACAAGCGCCAGCCTGGCTATTTCTGGACTGCAGAGCGAGGACGAGGCCGACTATTATTGT |
| | | GCCGCCTGGGATGCCCTGGACTGGGTCGTCGTTTTTGGCGGCGGAACAAAGCTGACCGTG |
| | | CTCGAGAGCAAATATGGACCACCATGCCCTCCATGTCCTTTCTGGGTGCTCGTGGTTGTT |
| | | GGCGGAGTGCTGGCCTGTTACAGCCTGCTGGTTACCGTGGCCTTCATCATCTTTTGGGTC |
| | | CGAAGCAAGCGGAGCAGACTGCTGCACAGCGACTACATGAACATGACCCCTAGACGGCCC |
| | | GGACCTACCAGAAAGCACTACCAGCCTTACGCTCCTCCTAGAGACTTCGCCGCCTACAGA |
| | | AGCCGCGTGAAGTTTTCTCGAAGTGCCGATGCTCCTGCATATCAGCAGGGACAGAACCAG |
| | | CTGTACAACGAGCTGAATCTGGGCCGGAGAGAGGAATACGACGTGCTGGATAAGAGGCGC |
| | | GGCAGAGACCCAGAAATGGGCGGGAAGCCACGACGGAAAAACCCCCAGGAGGGGCTGTAT |
| | | AATGAACTGCAGAAGGACAAAATGGCCGAGGCTTACAGCGAAATCGGGATGAAGGGAGAG |
| | | AGAAGGCGCGGAAAAGGCCACGATGGACTGTATCAGGGCCTGAGCACTGCCACCAAGGAC |
| | | ACCTACGATGCTCTGCACATGCAGGCACTGCCACCCAGGGGATCCGGAGAGGGCAGAGGA |
| | | AGTCTTCTAACATGCGGTGACGTGGAGGAGAATCCCGGCCCTATGGGTCGGGGGCTGCTC |
| | | AGGGGCCTGTGGCCGCTGCACATCGTCCTGTGGACGCGTATCGCCAGCACGATCCCACCG |
| | | CACGTTCAGAAGTCGGTTAATAACGACATGATAGTCACTGACAACAACGGTGCAGTCAAG |
| | | TTTCCACAACTGTGTAAATTTTGTGATGTGAGATTTTCCACCTGTGACAACCAGAAATCC |
| | | TGCATGAGCAACTGCAGCATCACCTCCATCTGTGAGAAGCCACAGGAAGTCTGTGTGGCT |
| | | GTATGGAGAAAGAATGACGAGAACATAACACTAGAGACAGTTTGCCATGACCCCAAGCTC |
| | | CCCTACCATGACTTTATTCTGGAAGATGCTGCTTCTCCAAAGTGCATTATGAAGGAAAAG |
| | | AAAAAGCCTGGTGAGACTTTCTTCATGTGTTCCTGTAGCTCTGATGAGTGCAATGACAAC |
| | | ATCATCTTCTCAGAAGAATATAACACCAGCAATCCTGACTTGTTGCTAGTCATATTTCAA |
| | | GTGACAGGCATCAGCCTCCTGCCACCACTGGGAGTTGCCATATCTGTCATCATCATCTTC |
| | | TACTGCTACCGCGTTAACCGGCAGTAA |
| 56 | 008LYG_D08 CD28Z | MPLLLLLPLLWAGALAQVQLVQSGAEVKKPGSSVKVSCKASGGTFSSYAISWVRQAPGQG |
| | CAR-T2A-Dominant- | LEWMGGIIPIFGTANYAQKFQGRVTITADESTSTAYMELSSLRSEDTAVYYCARGYQGMG |
| | negative TGFβ | VFDPWGQGTLVTVSSGGGGSGGGGSGGGGSAQSVLTQPPSASGTPGQRVTISCSGSSSNI |
| | receptor type 2 | GSNTVNWYQQLPGTAPKLLIYSNNQRPSGVPDRFSGSKSGTSASLAISGLQSEDEADYYC |
| | amino acid | AAWDALDWVVVFGGGTKLTVLESKYGPPCPPCPFWVLVVVGGVLACYSLLVTVAFIIFWV |
| | sequence | RSKRSRLLHSDYMNMTPRRPGPTRKHYQPYAPPRDFAAYRSRVKFSRSADAPAYQQGQNQ |
| | | LYNELNLGRREEYDVLDKRRGRDPEMGGKPRRKNPQEGLYNELQKDKMAEAYSEIGMKGE |
| | | RRRGKGHDGLYQGLSTATKDTYDALHMQALPPRGSGEGRGSLLTCGDVEENPGPMGRGLL |
| | | RGLWPLHIVLWTRIASTIPPHVQKSVNNDMIVTDNNGAVKFPQLCKFCDVRFSTCDNQKS |
| | | CMSNCSITSICEKPQEVCVAVWRKNDENITLETVCHDPKLPYHDFILEDAASPKCIMKEK |
| | | KKPGETFFMCSCSSDECNDNIIFSEEYNTSNPDLLLVIFQVTGISLLPPLGVAISVIIIF |
| | | YCYRVNRQ |
| 57 | Randomized CDR H3 | ARXXXXXXXXDX, where X can be any amino acid or optionally |
| | | present |
| 58 | Randomized CDR L3 | AAWDXXXXXVV, where X can be any amino acid or optionally |
| | | present |
| 59 | pSpliceFwd | CAGCTATGACCATGATTACGAATTT |
| 60 | pMcoFcRev | CTGATCATCAGGGTGTCCTTGG |
| 61 | P130s FOR | CCGTCGCCGCCACCATGGAC |
| 62 | P219 REV | CTAGAAGGCACAGTCGAGGC |
| 63 | Signal pep_intron | GGAGCTGTATCATCCTCTTC |
| | FOR | |
| 64 | P220 REV | GAGATGCTACTGGGGCAACGG |
| 65 | EF1 FOR | TTCGTTTTCTGTTCTGCGCCG |
| 66 | 4-1BB REV | TGTACAGCAGCTTCTTTCTGCC |
| 67 | CMS312s | AGCCGTACATGAACTGAGGG |
| 68 | 008LYG_D08 CD28z | MPLLLLLPLLWAGALAQVQLVQSGAEVKKPGSSVKVSCKASGGTFSSYAISWVRQAPGQG |
| | CAR-T2A-mCherry | LEWMGGIIPIFGTANYAQKFQGRVTITADESTSTAYMELSSLRSEDTAVYYCARGYQGMG |
| | | VFDPWGQGTLVTVSSGGGGSGGGGSGGGGSAQSVLTQPPSASGTPGQRVTISCSGSSSNI |
| | | GSNTVNWYQQLPGTAPKLLIYSNNQRPSGVPDRFSGSKSGTSASLAISGLQSEDEADYYC |
| | | AAWDALDWVVVFGGGTKLTVLESKYGPPCPPCPFWVLVVVGGVLACYSLLVTVAFIIFWV |
| | | RSKRSRLLHSDYMNMTPRRPGPTRKHYQPYAPPRDFAAYRSRVKFSRSADAPAYQQGQNQ |
| | | LYNELNLGRREEYDVLDKRRGRDPEMGGKPRRKNPQEGLYNELQKDKMAEAYSEIGMKGE |
| | | RRRGKGHDGLYQGLSTATKDTYDALHMQALPPREFEGSAAAEGRGSLLTCGDVEENPGPS |
| | | GMVSKGEEDNMAIIKEFMRFKVHMEGSVNGHEFEIEGEGEGRPYEGTQTAKLKVTKGGPL |
| | | PFAWDILSPQFMYGSKAYVKHPADIPDYLKLSFPEGFKWERVMNFEDGGVVTVTQDSSLQ |
| | | DGEFIYKVKLRGTNFPSDGPVMQKKTMGWEASSERMYPEDGALKGEIKQRLKLKDGGHYD |
| | | AEVKTTYKAKKPVQLPGAYNVNIKLDITSHNEDYTIVEQYERAEGRHSTGGMDELYK |
| 69 | P2A Linker | ATNFSLLKQAGDVEENPGP |
| 70 | E2A linker | QCTNYALLKLAGDVESNPGP |
| 71 | F2A linker | VKQTLNFDLLKLAGDVESNPGP |
| 72 | T2A linker | EGRGSLLTCGDVEENPGP |
| 73 | CD3z wild-type | RVKFSRSADAPAYQQGQNQLYNELNLGRREEYDVLDKRRGRDPEMGGKPQRRKNPQEGLY |
| | | NELQKDKMAEAYSEIGMKGERRRGKGHDGLYQGLSTATKDTYDALHMQALPPR |
| 74 | CD3z variant | RVKFSRSADAPAYQQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQALPPR |

The embodiments described herein can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein.

All patents and publications mentioned in this specification are herein incorporated by reference to the same extent as if each independent patent and publication was specifically and individually indicated to be incorporated by reference. Citation or identification of any reference in any section of this application shall not be construed as an admission that such reference is available as prior art to the present disclosure.

1-26. (canceled)

27. An anti-CLDN18.2 chimeric antigen receptor (CAR) comprising an antigen binding domain, wherein the antigen binding domain comprises an antibody, Fab, or an scFv comprising a heavy chain variable region (VH) and a light chain variable region (VL);
wherein the VH comprises a CDR1 comprising an amino acid sequence selected from SEQ ID NO: 1, 11, 21, 31, and 41; a CDR2 comprising an amino acid sequence selected from SEQ ID NO: 2, 12, 22, 32, and 42; and a CDR3 comprising an amino acid sequence selected from SEQ ID NO: 3 13, 23, 33, and 43; and
wherein the VL comprises a CDR1 comprising an amino acid sequence selected from SEQ ID NO: 4, 14, 24, 34, and 44; a CDR2 comprising an amino acid sequence selected from SEQ ID NO: 5, 15, 25, 35, and 45; and a CDR3 comprising an amino acid sequence selected from SEQ ID NO: 6, 16, 26, 36, and 46.

28. The anti-CLDN18.2 CAR of claim 27, wherein the VH comprises an amino acid sequence selected from SEQ ID NO: 7, 17, 27, 37 and 47.

29. The anti-CLDN18.2 CAR of claim 27, wherein the VL comprises an amino acid sequence selected from SEQ ID NO: 8, 18, 28, 38, and 48.

30. The anti-CLDN18.2 CAR of claim 27, wherein the CAR comprises a transmembrane domain, and one or more intracellular domains, and wherein the transmembrane domain comprises a transmembrane domain selected from the transmembrane domain of CD4, CD8α, or CD28.

31. (canceled)

32. The anti-CLDN18.2 CAR of claim 30, wherein the transmembrane domain comprises a CD28 transmembrane domain.

33. The anti-CLDN18.2 CAR of claim 27, wherein the one or more intracellular domains comprises a costimulatory domain or a portion thereof, and wherein the costimulatory domain comprises one or more of CD3z, CD2, CD27, CD28, 4-1BB, OX-40, ICOS, IL-2Rβ, GITR, MyD88/CD40a costimulatory domains, and/or variants thereof.

34. (canceled)

35. The anti-CLDN18.2 CAR of claim 33, wherein the intracellular domain comprises a CD3z costimulatory domain and a CD28 costimulatory domain.

36. (canceled)

37. (canceled)

38. The anti-CLDN18.2 CAR of claim 27, wherein the CAR further comprises a hinge/spacer domain, wherein the hinge/spacer domain comprises an IgG1 hinge domain or variants thereof, an IgG2 hinge domain or variants thereof, an IgG3 hinge domain or variants thereof, an IgG4 hinge domain or variants thereof, an IgG4P domain, a CD8a hinge domain or variants thereof, or a CD28 hinge domain or variants thereof.

39. (canceled)

40. The anti-CLDN18.2 CAR of claim 38, wherein the hinge/spacer domain is an IgG4 hinge/spacer, or variants thereof, optionally an IgG4P hinge/spacer comprising an S241P mutation.

41. The anti-CLDN18.2 CAR of claim 27, wherein the CAR has an amino acid sequence as set forth in SEQ ID NO: 52.

42. The anti-CLDN18.2 CAR of claim 27, wherein the CAR further comprises an armoring molecule.

43. The anti-CLDN18.2 CAR of claim 42, wherein the armoring molecule is selected from a dominant-negative TGFβ receptor type II, IL-7, IL-12, IL-15, IL-18, a hybrid IL-4/IL-7 receptor, a hybrid IL-7/IL-2 receptor, and HIF1α dominant-negative.

44. The anti-CLDN18.2 CAR of claim 43, wherein the armoring molecule comprises a dominant-negative TGFβ receptor type II (dnTGFβRII).

45. (canceled)

46. The anti-CLDN18.2 CAR of claim 44, wherein the dominant negative TGFβ receptor type II comprises the sequence of SEQ ID NO:54.

47. The anti-CLDN18.2 CAR of claim 42, wherein the CAR and the armoring molecule are linked by a nucleotide sequence encoding a cleavable peptide linker.

48. (canceled)

49. (canceled)

50. The anti-CLDN18.2 CAR of claim 42, wherein the CAR comprises SEQ ID NO: 56.

51. A vector comprising the isolated nucleic acid sequence encoding the chimeric antigen receptor of claim 27, optionally wherein the vector is a virus, a lentivirus, an adenovirus, a retrovirus, an adeno-associated virus (AAV), a transposon, a DNA vector, a mRNA, a lipid nanoparticle (LNP), or a CRISPR-Cas System, optionally wherein the vector is a lentivirus.

52. (canceled)

53. (canceled)

54. A cell comprising a CLDN18.2 specific antigen binding domain, wherein the antigen binding domain comprises an antibody, Fab, or an scFv comprising a heavy chain variable region (VH) and a light chain variable region (VL);
wherein the VH comprises a CDR1 comprising an amino acid sequence selected from SEQ ID NO: 1, 11, 21, 31, and 41; a CDR2 comprising an amino acid sequence selected from SEQ ID NO: 2, 12, 22, 32, and 42; and a CDR3 comprising an amino acid sequence selected from SEQ ID NO: 3 13, 23, 33, and 43; and
wherein the VL comprises a CDR1 comprising an amino acid sequence selected from SEQ ID NO: 4, 14, 24, 34, and 44; a CDR2 comprising an amino acid sequence selected from SEQ ID NO: 5, 15, 25, 35, and 45; and a CDR3 comprising an amino acid sequence selected from SEQ ID NO: 6, 16, 26, 36, and 46.

55-64. (canceled)

65. A method of treating cancer, comprising:
administering to a subject in need thereof an effective amount of a cell comprising an anti-CLDN18.2 chimeric antigen receptor (CAR) comprising an antigen binding domain, wherein the antigen binding domain comprises an antibody, Fab, or an scFv comprising a heavy chain variable region (VH) and a light chain variable region (VL),
wherein the VH comprises a CDR1 comprising an amino acid sequence selected from SEQ ID NO: 1, 11, 21, 31, and 41; a CDR2 comprising an amino acid sequence selected from SEQ ID NO: 2, 12, 22, 32, and 42; and a CDR3 comprising an amino acid sequence selected from SEQ ID NO: 3 13, 23, 33, and 43; and
wherein the VL comprises a CDR1 comprising an amino acid sequence selected from SEQ ID NO: 4, 14, 24, 34, and 44; a CDR2 comprising an amino acid sequence selected from SEQ ID NO: 5, 15, 25, 35, and 45; and a CDR3 comprising an amino acid sequence selected from SEQ ID NO: 6, 16, 26, 36, and 46.

66-68. (canceled)

69. The method of claim 65, wherein the cancer is a solid tumor, and wherein the solid tumor is gastric cancer, gastroesophageal junction cancer (GEJ, e.g., distal oesophageal cancers, proximal gastric cancers and cancers of cardia), or pancreatic cancer.

70-146. (canceled)
